SEQUENCE LISTING (833 sequences)

SEQ ID NO: 1-10 - skipped (10 entries)

SEQ ID NO: 11 - protein, 74 aa, Arabidopsis thaliana
CTSTLINGSSDEERTYSFSPTTSPFDPRSLNQELKIGRIGYCFDCARACMRRGKYIRTCSFERKLCRCSISDIK

SEQ ID NO: 12 - protein, 57 aa, Oryza sativa
  source 1..57: note = subspecies Japonica
GTTKVAEARHCLSQSHRFKGMCVSSNNCANVCRTESFPDGECKSHGLERKCFCKKVC

SEQ ID NO: 13 - protein, 108 aa, Artemisia vulgaris
AGSKLCEKTSKTYSGKCDNKKCDKKCIEWEKAQHGACHKREAGKESCFCYFDCSKSPPGATPAPPGAAPPPAAGGSPSPPADGGSPPPPADGGSPPVDGGSPPPPSTH

SEQ ID NO: 14 - protein, 50 aa, Arabidopsis thaliana
RICERRSKTWTGFCGNTRGCDSQCKRWERASHGACHAQFPGFACFCYFNC

SEQ ID NO: 15 - protein, 96 aa, Arabidopsis thaliana
SICNDRLGLCDGCDQRCKAKHGPSCESKCDGPVGMLLCTCTYECGPTKLCNGGLGNCGESCNEQCCDRNCAQRYNGGHGYCNTLDDFSLCLCKYPC

SEQ ID NO: 16 - protein, 46 aa, Arachis duranensis
KLCNHLADTYRGPCFTNASCDDHCKNKEHFVSGTCMKMACWCAHNC

SEQ ID NO: 17 - protein, 57 aa, Malus baccata
RTESSKAIEGKICEFPSTLFKGLCFSSNNCKHTCRKEQFTRGKCSLLTRTCMCTKKC

SEQ ID NO: 18 - protein, 55 aa, Oryza sativa
LAPAQSSVRPCETKSLRFEGFCMVEDNCANVCRTEGFVDGRCSTFVRNCICIKPC

SEQ ID NO: 19 - protein, 45 aa, Triticum dicoccoides
RDCTSQSHKFVGLCLSDRNCASVCLTEYFTGGKCDHRRCVCTKGC

SEQ ID NO: 20 - protein, 47 aa, unidentified
  source 1..47: note = Pyrus ussuriensis x Pyrus communis
KICEVPSTLFKGLCFSSNNCKHTCRKEQFTRGHCSVLTRACVCTKKC

SEQ ID NO: 21 - protein, 46 aa, Beta vulgaris
  source 1..46: note = subspecies Vulgaris
AICKKPSKFFKGACGRDADCEKACDQENWPGGVCVPFLRCECQRSC

SEQ ID NO: 22 - protein, 47 aa, Miscanthus lutarioriparius
RVCRRRIAGFKGVCMSDHNCAQVCLQEGWGGGNCDGILRQCKCIRQC

SEQ ID NO: 23 - protein, 103 aa, Lotus japonicus
NTCSEALDQCDSMESCDQKCKGRHNGRRGSCNLGLCTCSYSCGPPGPKIPSNLCTAGLGLCSTKCGDSYCNSICASKYNQGVGFCYTSGPSINLCTCQYPCAL

SEQ ID NO: 24 - protein, 49 aa, Aquilegia formosa
RDCESQSHKFKGTCIRKSNCASVCQSEGFNGGHCRGVTRRCYCTAKCHE

SEQ ID NO: 25 - protein, 49 aa, Dichanthelium oligosanthes
RHCLSPSHHFKGLCMSSNNCANVCRTENFPGGECKTEGATRKCMCKKVC

SEQ ID NO: 26 - protein, 56 aa, Arabis nemorensis
CTTSIGPCEVGKNCSPKCNAMFGEIVTGFCDRSGGGIGICVCVTPCPPPPPKAPHM

SEQ ID NO: 27 - protein, 99 aa, Sesamum indicum
AVCQTRSQQFRGLCFRNDNCAAICEKEGYISGSCKGFLLRCICFKDCGATGGGPPDGSPDGDGGTPEGLTGSHVTSNISSVYKYGKNEAKPGNAEIKKG

SEQ ID NO: 28 - protein, 45 aa, Brassica rapa
RTCESPSTKFQGVCLNAKNCAGVCTGEGFTGGQCSSLKCYCTKTC

SEQ ID NO: 29 - protein, 49 aa, Tetracentron sinense
KTCTKRSRTWFGLCLNSNGCNRQCKKWEGAKHGACHAQFQGTACFCYYC

SEQ ID NO: 30 - protein, 71 aa, Microthlaspi erraticum
RETCDKPYSICWFWPFFSDSDAYCRESCLKVKDEKYEYYDGQCRPARSALPLPPECFCCVRERSRAEIESI

SEQ ID NO: 31 - protein, 62 aa, Vanilla planifolia
RRCRAQSFTFKGICYSSHSCALVCKKEGYETGHCSRREQHKIFCICAFKSLNQTKDTLQELD

SEQ ID NO: 32 - protein, 47 aa, Glycine max
KDCLTRRHGFQGRCLFDRQCAHVCRSDGFIGGQCRGPLRKCFCSRPC

SEQ ID NO: 33 - protein, 49 aa, Brachypodium distachyon
RHCLSQSHKFKGTCVRSGNCANVCKTENFPDGECKTQGLERKCFCKRVC

SEQ ID NO: 34 - protein, 49 aa, Brachypodium distachyon
KLCEERSQKFRGPCKFNTNCVAVCVTEGYTGGYCHKTVFHSHCMCTKEC

SEQ ID NO: 35 - protein, 47 aa, Brachypodium distachyon
KICRKRSAGYHGMCMSDRNCAQVCQAEGWGGGNCDGAVRRCKCSREC

SEQ ID NO: 36 - protein, 47 aa, Brachypodium distachyon
RECETESTKFEGLCIMHSHCPDVCVTEGFTGGKCSTWKRKCMCTKEC

SEQ ID NO: 37 - protein, 48 aa, Brachypodium distachyon
KKCSKPSGRFKGPCFFKVDCEQQCKKEGWPRGVCTGFAGRCVCKNYGC

SEQ ID NO: 38 - protein, 146 aa, Brachypodium distachyon
YVHGFCNSPSGSFRGPCFSDSHCKAACEHEYYKGDKSKPFTDGKCSDSGSQRLDGVGVGSRWSLLPTCICSIFCGSEKITWRPIITIRLRCQKTQNRRLRTTITIRLRRQKAQNRRRRTITIRLRRRNKSRQHKAASRRGKCWETS

SEQ ID NO: 39 - protein, 103 aa, Brachypodium distachyon
LHWFCNIPSGSFHGPCMSNDHCLAACKSEYYNGDKSKPFTGGQCSLSGSILPRCICSIFCGPPMAEHDDPETHRHHHPPLPPPSEGPELSPPHHHAPPPRHHH

SEQ ID NO: 40 - protein, 47 aa, Brachypodium distachyon
RDCEAPSHKFHGACVRDSNCASVCETEGFSGDKREGPLMRCYCTKQC

SEQ ID NO: 41 - protein, 81 aa, Brachypodium distachyon
GDELLCDVRLTKCEDVCYKSGKCSRCCKNHRFNHGHCDLKSHCYCYRGPNSGAGDGEQQRMLRVIPASPPPPAARHPPLHA

SEQ ID NO: 42 - protein, 49 aa, Setaria italica
TLCNLRSMGFVGVCWSNMSCANQCVLEGRTSGYCKGIPAIKYCMCTFEC

SEQ ID NO: 43 - protein, 47 aa, Setaria italica
DRCETHSRTYKGRCNNHNCWSICITEGNTGGFCKGTLLLKCMCTSEC

SEQ ID NO: 44 - protein, 48 aa, Setaria italica
DMCKKRSRTFEGRCGLNMNCATVCVAEHYTGGFCKGFFHRECMCTKDC

SEQ ID NO: 45 - protein, 50 aa, Setaria italica
RRCVSQSHKFVGSCMRKSNCQHVCQTEGFPWGECRFHGGLLRRCFCNKLC

SEQ ID NO: 46 - protein, 29 aa, Arabidopsis thaliana
GYCFDCARACMRRGKYIRTCSFERKLCRC

SEQ ID NO: 47 - protein, 18 aa, Oryza sativa
  source 1..18: note = subspecies Japonica
GECKSHGLERKCFCKKVC

SEQ ID NO: 48 - protein, 19 aa, Artemisia vulgaris
GACHKREAGKESCFCYFDC

SEQ ID NO: 49 - protein, 18 aa, Arabidopsis thaliana
GACHAQFPGFACFCYFNC

SEQ ID NO: 50 - protein, 18 aa, Arabidopsis thaliana
GYCNTLDDFSLCLCKYPC

SEQ ID NO: 51 - protein, 14 aa, Arachis duranensis
GTCMKMACWCAHNC

SEQ ID NO: 52 - protein, 16 aa, Malus baccata
GKCSLLTRTCMCTKKC

SEQ ID NO: 53 - protein, 16 aa, Oryza sativa
GRCSTFVRNCICIKPC

SEQ ID NO: 54 - protein, 14 aa, Triticum dicoccoides
GKCDHRRCVCTKGC

SEQ ID NO: 55 - protein, 16 aa, unidentified
  source 1..16: note = Pyrus ussuriensis x Pyrus communis
GHCSVLTRACVCTKKC

SEQ ID NO: 56 - protein, 15 aa, Beta vulgaris
  source 1..15: note = subspecies Vulgaris
GVCVPFLRCECQRSC

SEQ ID NO: 57 - protein, 16 aa, Miscanthus lutarioriparius
GNCDGILRQCKCIRQC

SEQ ID NO: 58 - protein, 21 aa, Lotus japonicus
GFCYTSGPSINLCTCQYPCAL

SEQ ID NO: 59 - protein, 18 aa, Aquilegia formosa
GHCRGVTRRCYCTAKCHE

SEQ ID NO: 60 - protein, 18 aa, Dichanthelium oligosanthes
GECKTEGATRKCMCKKVC

SEQ ID NO: 61 - protein, 29 aa, Arabis nemorensis
GFCDRSGGGIGICVCVTPCPPPPPKAPHM

SEQ ID NO: 62 - protein, 16 aa, Sesamum indicum
GSCKGFLLRCICFKDC

SEQ ID NO: 63 - protein, 14 aa, Brassica rapa
GQCSSLKCYCTKTC

SEQ ID NO: 64 - protein, 17 aa, Tetracentron sinense
GACHAQFQGTACFCYYC

SEQ ID NO: 65 - protein, 18 aa, Microthlaspi erraticum
GQCRPARSALPLPPECFC

SEQ ID NO: 66 - protein, 15 aa, Vanilla planifolia
GHCSRREQHKIFCIC

SEQ ID NO: 67 - protein, 17 aa, Glycine max
GGQCRGPLRKCFCSRPC

SEQ ID NO: 68 - protein, 18 aa, Brachypodium distachyon
GECKTQGLERKCFCKRVC

SEQ ID NO: 69 - protein, 18 aa, Brachypodium distachyon
GYCHKTVFHSHCMCTKEC

SEQ ID NO: 70 - protein, 16 aa, Brachypodium distachyon
GNCDGAVRRCKCSREC

SEQ ID NO: 71 - protein, 16 aa, Brachypodium distachyon
GKCSTWKRKCMCTKEC

SEQ ID NO: 72 - protein, 17 aa, Brachypodium distachyon
GVCTGFAGRCVCKNYGC

SEQ ID NO: 73 - protein, 32 aa, Brachypodium distachyon
GKCSDSGSQRLDGVGVGSRWSLLPTCICSIFC

SEQ ID NO: 74 - protein, 19 aa, Brachypodium distachyon
GQCSLSGSILPRCICSIFC

SEQ ID NO: 75 - protein, 26 aa, Brachypodium distachyon
SVCETEGFSGDKREGPLMRCYCTKQC

SEQ ID NO: 76 - protein, 25 aa, Brachypodium distachyon
GKCSRCCKNHRFNHGHCDLKSHCYC

SEQ ID NO: 77 - protein, 18 aa, Setaria italica
GYCKGIPAIKYCMCTFEC

SEQ ID NO: 78 - protein, 17 aa, Setaria italica
GFCKGTLLLKCMCTSEC

SEQ ID NO: 79 - protein, 17 aa, Setaria italica
GFCKGFFHRECMCTKDC

SEQ ID NO: 80 - protein, 19 aa, Setaria italica
GECRFHGGLLRRCFCNKLC

SEQ ID NO: 81 - protein, 75 aa, Capsicum annuum
DICTTPPRRYIGVCWSDTSCRKVCVEMENFEDGYCSKIQRKCLCTWTCVLDNIPNDAGTIMLQDAKTMQLLEEKF

SEQ ID NO: 82 - protein, 75 aa, Capsicum annuum
YNICKTKSKYFEGLCWVDSSCRKVCIEKDKFEDGHCSKLLRNCLCTKICAFDNIPNDAGTILVQDAKSLEAQLLE

SEQ ID NO: 83 - protein, 75 aa, Capsicum annuum
QNNICKTTSKHFKGLCFADSKCRKVCIQEDKFEDGHCSKLQRKCLCTKNCVFDNIPNDVGTILVEDAKTLEAQLL

SEQ ID NO: 84 - protein, 75 aa, Capsicum annuum
QQTCKTESQKFKGLCITKSSCRKACLKEKFTDGHCSKLQRRCLCTKPCVFENNPNEVETKLVGKTKTLREAMLEE

SEQ ID NO: 85 - protein, 79 aa, Capsicum annuum
RQTCKTESQKFKGLCITKSSCRKACLKEKFTDGHCSKLQRRCLCTKPCVFENNPNEDETNLVGKTKTLREAMLEEQIME

SEQ ID NO: 86 - protein, 75 aa, Capsicum annuum
YNICKTKSKKFEGLCFLDSSCRKVCVEQENFEDGHCSKLRRLCLCTKLCVFDNIPNDVGTILVQDAKTLEAQLLE

SEQ ID NO: 87 - protein, 75 aa, Capsicum annuum
QNICKTKSKYFTGLCWTDSSCRKVCIEKDKFQDGHCSKLQRNCLCTKICVFDNIPNDAGTILVQDAKTNEAQLLE

SEQ ID NO: 88 - protein, 75 aa, Capsicum annuum; identical to SEQ ID NO: 82

SEQ ID NO: 89 - protein, 75 aa, Nicotiana tabacum
RECKTESNTFPGLCITKPPCRKACISEGFTDGHCSKILRRCLCTKPCVFDEKMIKTGAETLAEEAKTLAAALFEE

SEQ ID NO: 90 - protein, 75 aa, Nicotiana tabacum
RECKTESNTFPGLCITKPPCRKACISEGFTDGHCSKILRRCLCTKPCVFDEKMIKTGAETFAEEAKTLAAALLEE

SEQ ID NO: 91 - protein, 75 aa, Nicotiana tabacum
RECKAVSNTLSGLCITKPPCREACISEGFTDGKCSKILKRCICHKPCVFDEKMVKTGAETLAEEVKTLAAAFLEE

SEQ ID NO: 92 - protein, 75 aa, Nicotiana tabacum
RECKAESITFSGLCVSKPPCRKACICEGFTNGHCSKILRRCLCTTPSVFDEKMIKTGAETLGEEAKTLAATLLEE

SEQ ID NO: 93 - protein, 75 aa, Nicotiana tabacum
QSTCKAESNTFPGICITKPPCRKACLSEGFTDGKCSKILRRCICYKPCVFDEKMTKTGAETLAEEAKTLAAALLE

SEQ ID NO: 94 - protein, 75 aa, Nicotiana tabacum
RECKTESNTFPGICITKPPCRKACISEKFTDGHCSKLLRRCLCTKPCVFDEKMIKTGAETLVEEAKTLAAALLEE

SEQ ID NO: 95 - protein, 75 aa, Petunia hybrida
ATCKAECATWEGICINKAPCVKCCKEQPENFTDGHCSKILRRCLCTKPCATAEATATLANEVKTMGEALVEVDMM

SEQ ID NO: 96 - protein, 75 aa, Petunia hybrida
ATCKAECPTWDGICINKGPCVKCCKAQPEKFTDGHCSKVLRRCLCTKPCATEEATATLANEVKTMAEALVEEDMM

SEQ ID NO: 97 - protein, 75 aa, Petunia hybrida
ATCKAECPTWDSVCINKKPCVACCKKAKFSDGHCSKILRRCLCTKECVFEKTEATQTETFTKDVNTLAEALLEAD

SEQ ID NO: 98 - protein, 75 aa, Solanum lycopersicum
QQICKAPSQTFPGLCFMDSSCRKYCIKEKFTGGHCSKLQRKCLCTKPCVFDKISSEVKATLGEEAKTLSEVVLEE

SEQ ID NO: 99 - protein, 68 aa, Solanum lycopersicum
QPMCKSTSQTFKGLCFTDSSCRKSCLKEEFEGGHCSKLQRKCLCTKICVFDKISNEVRTNFGWGSKNS

SEQ ID NO: 100 - protein, 78 aa, Solanum lycopersicum
ASSTRANQQAKSSKDYVLPTHHVEKLVSKRSLKVDIIFKLQSKCLCTKICVFEKNPNEVKPTLGGEAKTLSETMLEDD

SEQ ID NO: 101 - protein, 62 aa, Solanum lycopersicum
QHMCKSTSQTFKGLCFTYSSCRKGCLKEKFEGGHYIVANSKGSAYALRFVYLTKFQMKLNQL

SEQ ID NO: 102 - protein, 75 aa, Solanum lycopersicum
QYMCKSTSQTFKGLCFTDSSCRKACVTEEFTGGHCSKLQRKCLCTKVCVLEKDSNEVKTTLVGEAKTLSETVLEE

SEQ ID NO: 103 - protein, 75 aa, Solanum lycopersicum
QQMCKSTSQTFKGLCFTDSSCRKACVTEEFTGGHCSKLQRKCLCTKVCVFEKDSNEVKTTLVGEAKTLSETVLEE

SEQ ID NO: 104 - protein, 75 aa, Solanum pennellii
RQICKAPSQTFPGLCFMDSSCRKYCIKEKFTGGHCSTLQRRCLCTKPCVFDIISNEVKATLGEEAKTLSEVVLEE

SEQ ID NO: 105 - protein, 75 aa, Solanum pennellii
QQMCKSTSQTFKGLCFIDSSCRKACVTEEFTGGHCSKLQRKCLCTKVCVFEKDSNEVKTTLVGEAKTLSETVLEE

SEQ ID NO: 106 - protein, 49 aa, Cajanus cajan
AVCSKPSTYFFGPCVRRSTCRRACSHENYPDGKCSRFLGKCICYKPCTT

SEQ ID NO: 107 - protein, 46 aa, Eucalyptus grandis
ATCRKPSMYFSGACFSDTNCQKACNREDWPNGKCLVGFKCECQRPC

SEQ ID NO: 108 - protein, 55 aa, Eucalyptus grandis
MNIVTKVDGAICKKPSKFFKGACGRDADCEKACDQENWPGGVCVPFLRCECQRSC

SEQ ID NO: 109 - protein, 49 aa, Eucalyptus grandis
ATCAKPSKYYKGGCFATISVSACKKTCSRENWPDGACVYPFRCECRRPC

SEQ ID NO: 110 - protein, 49 aa, Eucalyptus grandis
KTCAKPSKYYKGGCFSTISVSACKKTCSRENWPDGDCVFPYRCECRRPC

SEQ ID NO: 111 - protein, 49 aa, Eucalyptus grandis
ANCAKPSKFYRGPCFNSISVKPCTQRCSQENWPIGVCTKDLRCECQRPC

SEQ ID NO: 112 - protein, 49 aa, Eucalyptus grandis
FSVISDSACKKACNQENWPIGICVPVFRCECRRSCQAMFSPESSSIKYE

SEQ ID NO: 113 - protein, 49 aa, Brassica napus
KARMCETSSQLFNGPCLSTTNCANICQNEGFPDGDCKGFRLRCICNRPC

SEQ ID NO: 114 - protein, 47 aa, Brassica rapa
RTCESQSHRFKGPCVSENNCANVCHNEGFGGGKCRGLRRRCFCTRHC

SEQ ID NO: 115 - protein, 50 aa, Brassica napus
IEGRMCQSRSHHFRSICLSNHNCANICRGERFSGGRCHGLHRKCYCTRLC

SEQ ID NO: 116 - protein, 45 aa, Brassica rapa; identical to SEQ ID NO: 28

SEQ ID NO: 117 - protein, 47 aa, Brassica cretica
RTCESKSHRFKGTCVSSTNCGNVCHNEGFGGGKCRGFRRRCYCTRHC

SEQ ID NO: 118 - protein, 47 aa, Brassica napus
RTCESKSHKFRGPCVSRHNCANVCHNEGFHGGKCRGFRRRCYCTRHC

SEQ ID NO: 119 - protein, 48 aa, Zostera marina
RTCVSKSEKFKGVCLINTNCAHVCQTEGFPDGDCRGSVRRRCFCIRPC

SEQ ID NO: 120 - protein, 47 aa, Microthlaspi erraticum
RTCESKSHRFKGPCVSTHNCGNVCHNEGFGGGKCRGFRRRCYCTRHC

SEQ ID NO: 121 - protein, 47 aa, Arabidopsis lyrata
RTCASKSEKFKGSCVSNTNCANICHTEGFPEGACRGLRHHCFCTRPC

SEQ ID NO: 122 - protein, 53 aa, Thlaspi arvense
QVRMCESKSHEFKGFCTKWRICRDVCITEGFTDGGCHGVLRHCVCKKPCLLSN

SEQ ID NO: 123 - protein, 54 aa, Actinidia chinensis
MIKQGEARVCESKSHGFKGACLIDHNCALVCRNEGFSGGKCRGFRHRCFCTKIC

SEQ ID NO: 124 - protein, 63 aa, Actinidia chinensis
KLCTAPSSTFKYLCISDQNCSSACEQEGFTSGECEGLRRRCICGKPCGMEVSKTKFDNGIGGE

SEQ ID NO: 125 - protein, 47 aa, Arabidopsis lyrata
RTCETSSNLFNGPCLSSSNCANVCHNEGFSDGDCRGFRRRCLCTRPC

SEQ ID NO: 126 - protein, 54 aa, Arabidopsis lyrata
QPQLCETKSLNYRGLCMKWRNCKRVCISEGFPDGRCKGFFNNKCVCRKPCALST

SEQ ID NO: 127 - protein, 47 aa, Arabidopsis lyrata
RTCASQSQRFKGKCVSDTNCANVCHNEGFPGGDCRGFRRRCFCTRNC

SEQ ID NO: 128 - protein, 47 aa, Arabidopsis lyrata
RTCESKSHRFKGPCVSTHNCANVCHNEGFGGGKCRGFRRRCYCTRHC

SEQ ID NO: 129 - protein, 45 aa, Arabidopsis lyrata
RTCESPSNKFQGVCLNSQSCAKACPSEGFSGGRCSSLRCYCSKAC

SEQ ID NO: 130 - protein, 59 aa, Arabidopsis lyrata
EVSPVDNKICKTRSDRFSGVCLSTNNCAIICQQFEHFDGGHCEFDGALRRCMCTKQCNN

SEQ ID NO: 131 - protein, 54 aa, Arabidopsis lyrata; identical to SEQ ID NO: 126

SEQ ID NO: 132 - protein, 52 aa, Arabidopsis lyrata
QQMCETQSINFRGMCTKWRNCQRVCMSEGFPDGRCKGFIRKCICTKPCPVST

SEQ ID NO: 133 - protein, 53 aa, Arabidopsis lyrata
IIGSDGRTCQSKSHHFKYMCTSSHNCAIVCRNEGFSGGRCHGFHRRCYCTRLC

SEQ ID NO: 134 - protein, 49 aa, Aquilegia oxysepala; identical to SEQ ID NO: 24

SEQ ID NO: 135 - protein, 49 aa, Arachis hypogaea
GGRHCESKSHRFKGMCVHERNCASVCNLEGFSGGKCRGFRRRCFCTRHC

SEQ ID NO: 136 - protein, 63 aa, Arachis hypogaea
EGRKCDSQSHHFKGKCFSDTNCASVCHGSFIHVFVELDLQSFEFTSCFSLEIKKFSSRSGQLF

SEQ ID NO: 137 - protein, 48 aa, Arachis hypogaea
RTCASQSHRFKGVCLSDTNCASVCKTEGFPSGDCHGFRRRCFCTKHCA

SEQ ID NO: 138 - protein, 55 aa, Arachis hypogaea
RTCASQSHRFKGVCLSDTNCASVCKTEGFPSGDCHGFRRRCFCTKHYRKIRSQDS

SEQ ID NO: 139 - protein, 49 aa, Arachis hypogaea
EGRHCESKSHRFKGMCVHERNCASVCNLEGFSGGKCRGFRRRCFCTRHC

SEQ ID NO: 140 - protein, 47 aa, Arachis hypogaea
RVCQSKSHMFKGGCWGDHNCALVCRNEGFSGGRCRGFRRRCFCTKLC

SEQ ID NO: 141 - protein, 47 aa, Arabidopsis thaliana; identical to SEQ ID NO: 125

SEQ ID NO: 142 - protein, 47 aa, Arabidopsis thaliana
RTCESQSHRFKGTCVSASNCANVCHNEGFVGGNCRGFRRRCFCTRHC

SEQ ID NO: 143 - protein, 47 aa, Arabidopsis thaliana
RTCASQSQRFKGKCVSDTNCENVCHNEGFPGGDCRGFRRRCFCTRNC

SEQ ID NO: 144 - protein, 47 aa, Arabidopsis thaliana; identical to SEQ ID NO: 128

SEQ ID NO: 145 - protein, 45 aa, Arabidopsis thaliana; identical to SEQ ID NO: 129

SEQ ID NO: 146 - protein, 57 aa, Arabidopsis thaliana
EVSPLDNKICKTRSDRFSGVCISTNNCAIICQQFEHFDGGHCEFDGAFRRCMCTKQC

SEQ ID NO: 147 - protein, 57 aa, Arabidopsis thaliana
QPQLCETKSLNYRGLCLKWRSCKRVCISEGFPDGRCKGFFNNKCVCRKPCALLSTEN

SEQ ID NO: 148 - protein, 53 aa, Arabidopsis thaliana
QQMCEAKSLDWKGMCLKWRNCRQVCISEGFTDGRCKGFTRKCICSKPCFVLPN

SEQ ID NO: 149 - protein, 47 aa, Arabidopsis thaliana
RTCQSKSHHFKYMCTSNHNCAIVCRNEGFSGGRCHGFHRRCYCTRLC

SEQ ID NO: 150 - protein, 58 aa, Acer truncatum
EMGQNQMVAEARTCESQSHKFKGTCVSKSNCAHVCQTEGFPGGHCRGFRRRCFCAKNC

SEQ ID NO: 151 - protein, 55 aa, Acer truncatum
QNEMVAEARTCESQSHKFKGTCLSKSNCANVCKTEGFPGGHCRGFRRRCFCTKKC

SEQ ID NO: 152 - protein, 49 aa, Brassica carinata
KARMCETSSQLFNGPCLSTTNCANICQNEGFPDGDCHGFRLRCICNRPC

SEQ ID NO: 153 - protein, 47 aa, Brassica carinata
RTCESQSHRFKGTCVSENNCANVCHNEGFGGGKCRGFRRRCFCTRNC

SEQ ID NO: 154 - protein, 75 aa, Brassica carinata
MDPVTVEARTCESKSHKFRGLCVSTHNCANVCHTKVKRSKECVSTDRTVPLLVPKKVPQAVVAVVSVAPAPNPAS

SEQ ID NO: 155 - protein, 47 aa, Brassica carinata; identical to SEQ ID NO: 118

SEQ ID NO: 156 - protein, 47 aa, Brassica carinata
RTCESQSHRFKGPCVSDNNCANVCHNEGFGGGKCRGLRRRCFCTTHC

SEQ ID NO: 157 - protein, 47 aa, Brassica carinata
RTCESKSHKFRGLCVSRHNCANVCHNEGFHGGKCRGFRRRCYCTRHC

SEQ ID NO: 158 - protein, 47 aa, Brassica carinata
RTCESKSHRFKGTCVSTTNCANVCHNEGFPGGKCRGFRRRCFCTRHC

SEQ ID NO: 159 - protein, 47 aa, Brassica carinata; identical to SEQ ID NO: 117

SEQ ID NO: 160 - protein, 49 aa, Brassica carinata; identical to SEQ ID NO: 113

SEQ ID NO: 161 - protein, 49 aa, Brassica oleracea; identical to SEQ ID NO: 113

SEQ ID NO: 162 - protein, 47 aa, Brassica oleracea
RTCESQSHRFKGPCVSDNNCANVCHNEGFGGGKCRGLRRRCFCTRHC

SEQ ID NO: 163 - protein, 50 aa, Brassica oleracea; identical to SEQ ID NO: 115

SEQ ID NO: 164 - protein, 72 aa, Brassica oleracea
QQMCEAKSLNFKGMCLKWKNCKHVFFTRKQLIKLSKKDVAAAEEEKILKETCMKLVQGQQMCEAKSINFKGM

SEQ ID NO: 165 - protein, 47 aa, Brassica oleracea; identical to SEQ ID NO: 117

SEQ ID NO: 166 - protein, 47 aa, Brassica oleracea; identical to SEQ ID NO: 157

SEQ ID NO: 167 - protein, 49 aa, Brassica rapa; identical to SEQ ID NO: 113

SEQ ID NO: 168 - protein, 47 aa, Brassica rapa; identical to SEQ ID NO: 114

SEQ ID NO: 169 - protein, 47 aa, Brassica rapa
RMCQSRSHHFRSICLSNHNCANICRGERFSGGRCHGLHRKCYCTRLC

SEQ ID NO: 170 - protein, 47 aa, Brassica rapa; identical to SEQ ID NO: 117

SEQ ID NO: 171 - protein, 47 aa, Brassica rapa; identical to SEQ ID NO: 118

SEQ ID NO: 172 - protein, 49 aa, Corchorus olitorius; identical to SEQ ID NO: 113

SEQ ID NO: 173 - protein, 47 aa, Corchorus olitorius; identical to SEQ ID NO: 162

SEQ ID NO: 174 - protein, 50 aa, Corchorus olitorius; identical to SEQ ID NO: 115

SEQ ID NO: 175 - protein, 72 aa, Corchorus olitorius
QQMCEAKSINFKGMSLKWKNCKHFIHENELIKLSRKEVAAAEEEKFLKETCMKLVQGQQMCEAKSINFKGIC

SEQ ID NO: 176 - protein, 47 aa, Corchorus olitorius; identical to SEQ ID NO: 117

SEQ ID NO: 177 - protein, 45 aa, Corchorus olitorius; identical to SEQ ID NO: 28

SEQ ID NO: 178 - protein, 47 aa, Corchorus olitorius; identical to SEQ ID NO: 157

SEQ ID NO: 179 - protein, 48 aa, Capsicum annuum
RSCESASQRFNGYCVRSSNCASICSTEGFTDGKCKGTIRRHCTCIRNC

SEQ ID NO: 180 - protein, 48 aa, Capsicum annuum
RVCISQSHGFKGPCGHDHNCALVCRNEGFSGGDCIGVFIRKCYCTKNC

SEQ ID NO: 181 - protein, 48 aa, Capsicum annuum
KICEALSGNFKGLCLSSRDCGNVCRREGFTDGSCIGFRLQCFCTKPCA

SEQ ID NO: 182 - protein, 47 aa, Capsicum annuum
RKCESQSHRFKGPCVRKSNCASVCQTEGFTGGHCRGFRRRCFCTKHC

SEQ ID NO: 183 - protein, 47 aa, Capsicum annuum
RHCESKSQRFKGPCVRQKNCAAVCETEGFPGGDCRGFRRRCFCTRPC

SEQ ID NO: 184 - protein, 47 aa, Capsicum annuum
RTCESQSHRFKGVCASETNCASVCQTEGFSGGDCRGFRRRCFCTRPC

SEQ ID NO: 185 - protein, 51 aa, Capsicum annuum
KTCDTLSENFKGICFSSTNCATTCQNEDEEYTGGHCRGLHRRCFCTTECEL

SEQ ID NO: 186 - protein, 49 aa, Capsicum annuum
KTCERLSGTFKGICFSSKNCASVCENEDEEFTGGHCSGLRRRCYCTADC

SEQ ID NO: 187 - protein, 47 aa, Cannabis sativa
RVCESKSHHFKGLCVIDHNCATICRTEGFSGGNCQGFRQRCFCTRHC

SEQ ID NO: 188 - protein, 47 aa, Cannabis sativa
RTCEAQSHHFKGPCVRKSNCANVCRTEGFPSGHCRGFRRRCFCTKHC

SEQ ID NO: 189 - protein, 47 aa, Cannabis sativa
KTCEARSQSHRGICVTNHTCDNVCKIERFSSGYCSGIDRRCLCTKHC

SEQ ID NO: 190 - protein, 47 aa, Cardamine hirsuta
RTCQSSSQLFSGPRLSTSNCANVCQNEGFTDGDCRGFRRRCFCNKQC

SEQ ID NO: 191 - protein, 54 aa, Cardamine hirsuta
QQLCETKSTNYRGLCLRWRNCKRVCMSEGFPDGRCRGIFDNKCVCRKPCALSTA

SEQ ID NO: 192 - protein, 47 aa, Cardamine hirsuta
RTCESQSHRFKGTCVSASNCGNVCHNEGFNGGNCRGFRRRCFCTRHC

SEQ ID NO: 193 - protein, 47 aa, Cardamine hirsuta
RTCESQSHRFKGTCTNEQNCRNICHNEGFFGGKCRGFRRRCFCTRNC

SEQ ID NO: 194 - protein, 47 aa, Cardamine hirsuta
RMCESKSQQFRGLCMSSTNCATVCHNEGFPGGECRGFRRRCFCTRPC

SEQ ID NO: 195 - protein, 100 aa, Cardamine hirsuta
KPRRCESQSNKFKGKCVSETNCGNVCRTEGFTGGDCRGFRRRCFCTRNCCARHRMKLSMRLISAALLLLMLFVATGMGPVMVDARTCESKSHRFKGPCVS

SEQ ID NO: 196 - protein, 47 aa, Cardamine hirsuta
RVCETRSQKFKGPCVSENNCANVCQTEGFPDGDCNGILRRCYCNSPC

SEQ ID NO: 197 - protein, 45 aa, Cardamine hirsuta
KTCETPSTKFQGVCLNSQNCAKVCPTEGASGGRCNSLRCVCSKTC

SEQ ID NO: 198 - protein, 47 aa, Cardamine hirsuta
RICQSKSHHYKYMCTSNHNCAMVCRNEGFSGGQCHGFHRRCYCTRLC

SEQ ID NO: 199 - protein, 47 aa, Capsella rubella
RTCESSSNLFSGPCLSESNCANVCNTEGFPDGDCRGFRRRCMCTRPC

SEQ ID NO: 200 - protein, 47 aa, Capsella rubella
RTCESKSHRFKGPCVSGNNCKNVCHNEGFPGGRCRGFRRRCYCTRHC

SEQ ID NO: 201 - protein, 45 aa, Capsella rubella
RTCESPSSKFSGVCLNSQTCAKACPSEGFTGGRCNSLRCYCSKPC

SEQ ID NO: 202 - protein, 57 aa, Capsella rubella
EMGLAADKVCKTRSDRFSGVCLSNNNCAIICQQFENFDSGHCEFDGAFRRCLCTKNC

SEQ ID NO: 203 - protein, 47 aa, Capsella rubella
RMCQSKSHHFRRMCWSDHNCAMICRSERFSGGHCHGFHRRCYCTRLC

SEQ ID NO: 204 - protein, 47 aa, Cicer arietinum
RVCASQSHAFKGPCVGDHNCALVCRNEGFSGGNCQGFRRRCFCTKLC

SEQ ID NO: 205 - protein, 47 aa, Citrus clementina
RTCESLSHRFKGTCVRGSNCAAVCQTEGFPGGQCSGLRRRCFCSKPC

SEQ ID NO: 206 - protein, 77 aa, Citrus clementina
HDTEMGPVLCGDTGNEKGPSRRKAAAVAAIERTCETPSKRFKGPCVKESNCAAVCQTEGFQGGKCRGLRRRCFCTRP

SEQ ID NO: 207 - protein, 47 aa, Citrus clementina
RICESQSHRFKGPCIRKDNCAAVCQTEGFHGGRCRGFRRRCFCTKHC

SEQ ID NO: 208 - protein, 47 aa, Ceratophyllum demersum
RMCESPSRHYKGLCFRSSNCASICHIEHFSHGHCHGFRRRCICSRPC

SEQ ID NO: 209 - protein, 47 aa, Ceratophyllum demersum
RMCESPSRHYKGLCFRSSNCASICHIEHFSHGYCHGFRRRCICSRPC

SEQ ID NO: 210 - protein, 47 aa, Carpinus fangiana
RVCESQSHKFHGPCLGDHNCALVCRNEGFSGGDCKGLRRRCFCTRLC

SEQ ID NO: 211 - protein, 50 aa, Citrullus lanatus
AEARICESQSHRFKGPCVSKSNCAAVCQTEGFHGGHCRGFRRRCFCTKRC

SEQ ID NO: 212 - protein, 47 aa, Citrullus lanatus
RVCQSQSHHFHGACFSHHNCAFVCRNEGFSGGKCRGARRRCFCSKLC

SEQ ID NO: 213 - protein, 47 aa, Carya illinoinensis
RVCESKSHGFQGMCTRDHNCALVCRNEGFSGGKCRGFRHRCFCTKLC

SEQ ID NO: 214 - protein, 47 aa, Carya illinoinensis
RVCESKSHKFQGPCVRDNNCGLVCKNEGFTGGKCRGFRRRCFCTKIC

SEQ ID NO: 215 - protein, 58 aa, Citrullus lanatus
EEMMGGGRAEGRVCKSQSHSFHGPCMRDHNCAVVCRTEGFSGGDCVGFRRRCFCTRLC

SEQ ID NO: 216 - protein, 47 aa, Corchorus olitorius
RVCESKSHWFKGPCWGDHNCALVCRNEGFSGGRCRGFRHRCFCTKLC

SEQ ID NO: 217 - protein, 47 aa, Corchorus olitorius
RLCQSQSHGFKGGCLSNHNCALVCRNEGFSGGQCRGLRHRCFCTRRC

SEQ ID NO: 218 - protein, 32 aa, Carica papaya
RTCQSQSHGFHGACLSHHNCAVVCQKEGFSGX

SEQ ID NO: 219 - protein, 47 aa, Carica papaya
RTCESQSHRFKGACGSRSNCAAVCQTEGFHGGHCRGFRRRCFCTKHC

SEQ ID NO: 220 - protein, 50 aa, Cucumis sativus
VEGRICESKSHGFKGPCMTDHNCALVCRNEGFSGGKCRGVRHRCFCTKLC

SEQ ID NO: 221 - protein, 50 aa, Cucumis sativus; identical to SEQ ID NO: 220

SEQ ID NO: 222 - protein, 73 aa, Daucus carota
TDTCEVRSAYFVSLCLIDHHCEMVCKVEGFPTGKCLGLVPHCICLGTCKQPSPPSPPPANPPESPPPVPPPTP

SEQ ID NO: 223 - protein, 47 aa, Daucus carota
RTCESQSHKFKGTCLSETNCGNVCKNEGFSGGNCRGLRRRCFCTRHC

SEQ ID NO: 224 - protein, 47 aa, Daucus carota
RTCESQSHKFKGTCVSRSNCANVCKNEGFPGGHCRGFRRRCFCIKHC

SEQ ID NO: 225 - protein, 47 aa, Davidia involucrata
RVCEAQSHGFKGACMSNHNCALVCRNEGFSGGKCRGFRHRCFCTKQC

SEQ ID NO: 226 - protein, 47 aa, Durio zibethinus
RVCESKSHRFKGPCLSHHNCGMVCKNEGFSGGKCRGVRRRCFCTRRC

SEQ ID NO: 227 - protein, 52 aa, Durio zibethinus
ISVEGRTCESQSHRFKGACVSENNCAAVCQTEGFLGGNCRGFRRRCFCTKQC

SEQ ID NO: 228 - protein, 52 aa, Durio zibethinus
IPLEGRTCESQSHRFKGMCVRESNCATVCQTEGFQGGHCRGFHRRCFCTKHC

SEQ ID NO: 229 - protein, 47 aa, Durio zibethinus
RTCESQSHRFKGTCLRKSNCAAVCQTEGFHGGHCRGFRRRCFCTKHC

SEQ ID NO: 230 - protein, 50 aa, Durio zibethinus
GEARVCESKSHKYKGACFHDHNCAYVCRNEGFSGGKCHGLHHHCFCTKIC

SEQ ID NO: 231 - protein, 47 aa, Durio zibethinus
RVCESKSHKFKGPCMGDHNCAYVCRNEGFSGGKCRGLRHRCFCTKIC

SEQ ID NO: 232 - protein, 47 aa, Erigeron canadensis
RMCQSQSHMYHGACLRDHNCALVCRNEGFSGGRCRGFRRRCFCTRLC

SEQ ID NO: 233 - protein, 71 aa, Erigeron canadensis
NEMGGPMVVEGRTLAVVTVIGAGGVQRWWRWWQCNGDSGGDGGTRAMVAVTNSGGSSSNGGGGDDWWWWWQ

SEQ ID NO: 234 - protein, 48 aa, Eutrema salsugineum
RMCTNPSKTYKGPCVSDTNCESVCQSEGFPQGNCHGFRRRCICGKPCA

SEQ ID NO: 235 - protein, 47 aa, Eutrema salsugineum
RTCETASHKFKGPCLGDRNCANVCQTEGFPGGDCQGLRRRCFCTRPC

SEQ ID NO: 236 - protein, 48 aa, Eutrema salsugineum
RTCDAPSHSFKGICTRKRNCESVCNSEGFADGDCKGFRRRCICNRPCA

SEQ ID NO: 237 - protein, 49 aa, Eutrema salsugineum
RTCVTPSHRFRGICVSKRNCESACNSEGFPDGNCKGFRRRCICNRPCAK

SEQ ID NO: 238 - protein, 51 aa, Eutrema salsugineum
LFGRCKKVPSRTYKGPCARDANCRSVCISEGYNGGDCHGFRRRCMCTDKCS

SEQ ID NO: 239 - protein, 47 aa, Eucalyptus grandis
RTCESQSQRFKGACVSKTNCASVCQTEGFHGGHCRGFRRRCFCTKHC

SEQ ID NO: 240 - protein, 50 aa, Eucalyptus grandis
IEGRMCESQSHKFHGLCMSDRTCVVVCKEEDFDDGKCRGFRHRCFCRKPC

SEQ ID NO: 241 - protein, 47 aa, Eucalyptus grandis
RVCESQSHGFHGSCVSGHNCALVCRNEGFSGGRCRGFRRRCFCTKLC

SEQ ID NO: 242 - protein, 47 aa, Fragaria X ananassa
RVCESQSHGFHGTCIRAHNCALVCRHEGFSGGRCRGFRRRCFCTRRC

SEQ ID NO: 243 - protein, 47 aa, Fragaria X ananassa
RTCETLSHKFKGTCLRESNCASVCQTEGFTGGDCRGLRRRCFCTKHC

SEQ ID NO: 244 - protein, 47 aa, Fragaria X ananassa
RVCESQSHGFYGTCIHAHNCALVCRHEGFSGGRCRGFRRRCFCTRRC

SEQ ID NO: 245 - protein, 47 aa, Fragaria X ananassa
AICKVPSKTFKGMCFSQKNCIVRCHSEGYGWGECSHLRRRCMCYKPC

SEQ ID NO: 246 - protein, 47 aa, Fragaria X ananassa
RVCEVPSKTFTGLCFRDTHCIVRCHSEGYGYGKCSHVLRRCRCLKAC

SEQ ID NO: 247 - protein, 47 aa, Fragaria X ananassa; identical to SEQ ID NO: 245

SEQ ID NO: 248 - protein, 47 aa, Fragaria X ananassa; identical to SEQ ID NO: 242

SEQ ID NO: 249 - protein, 53 aa, Fragaria X ananassa
GRPDPKTCESPSAKFEGPCIMGSNCALTCQKEGHDGGFCRDFWLPKCFCTKPC

SEQ ID NO: 250 - protein, 47 aa, Fragaria X ananassa
RTCESLSHKFQGTCLRESNCASVCQTEGFTGGDCRGLRRRCFCTKHC

SEQ ID NO: 251 - protein, 53 aa, Fragaria X ananassa
GRPDPKTCEPPSAKFEEPCIMGSNCALTCQKEGHDGGFCHDFWLPKCFCTKPC

SEQ ID NO: 252 - protein, 75 aa, Fragaria X ananassa
LGPKVMVAHEAITCETQSIKYEGMCSREKPCASLCQTKGYSRGECHEVITLRLLYRPGKTDPIGQSKRRTYPLTN

SEQ ID NO: 253 - protein, 47 aa, Fragaria X ananassa; identical to SEQ ID NO: 246

SEQ ID NO: 254 - protein, 53 aa, Fragaria X ananassa
GRPDPKTCESPSAKFEGPCIMGSNCALTCQKEGHNGGFCRDFWLPKCFCTKPC

SEQ ID NO: 255 - protein, 53 aa, Fragaria X ananassa
GRPDPKTCESPSGKFEGPCIMGSNCALTCQKEGHDGGFCRDFWLPKCFCTKPC

SEQ ID NO: 256 - protein, 53 aa, Fragaria vesca; identical to SEQ ID NO: 249

SEQ ID NO: 257 - protein, 58 aa, Fragaria vesca
LGPKVMVAHEAITCETQSIKYEGMCSREKPCASLCQTKGYSRGECHGVYKICVCATQC

SEQ ID NO: 258 - protein, 47 aa, Fragaria vesca; identical to SEQ ID NO: 245

SEQ ID NO: 259 - protein, 48 aa, Fragaria vesca
SICEIPSKKFKGLCIRDRKCIVICHSEGYGYGKCSHIGRRCMCLKTCD

SEQ ID NO: 260 - protein, 54 aa, Fragaria vesca
MVAADDARKCESLSHNFNGLCLSESNCASVCATEGFTGGDCRGLRRRCFCTKQC

SEQ ID NO: 261 - protein, 47 aa, Fragaria vesca; identical to SEQ ID NO: 243

SEQ ID NO: 262 - protein, 47 aa, Fragaria vesca
RTCESLSHKFKGTCLSETNCASVCKTEGFSGGDCRGLRRRCFCTKHC

SEQ ID NO: 263 - protein, 47 aa, Fragaria vesca; identical to SEQ ID NO: 242

SEQ ID NO: 264 - protein, 45 aa, Glycine max
MHLRSHRFKGMCLSEHNCASVSHLEGFTGGKCWGFRRRCFCSKHC

SEQ ID NO: 265 - protein, 47 aa, Glycine max; identical to SEQ ID NO: 32

SEQ ID NO: 266 - protein, 47 aa, Glycine max
RVCESQSHGFHGLCNRDHNCALVCRNEGFSGGRCKGFRRRCFCTRIC

SEQ ID NO: 267 - protein, 47 aa, Glycine max
RHCESKSHRFKGMCLSKHNCASVCHLEGFTGGKCRGFRRRCFCTRHC

SEQ ID NO: 268 - protein, 63 aa, Glycine max
RYIYIIPYVAVKRAEGRDCSSKSHGFKGLCFSDTNCAHVCRTEGFTGGDCGGFRRRCFCTRIC

SEQ ID NO: 269 - protein, 69 aa, Glycine max
RTCESQSHRFKGPCLSDTNCGSVCRTEGFSGGHCRGFSRRCFCTKEYDNYYKSSDVIRCKDGSGKFTKA

SEQ ID NO: 270 - protein, 47 aa, Glycine max
RTCESQSHRFKGPCLSDTNCGSVCRTERFTGGHCRGFRRRCFCTKHC

SEQ ID NO: 271 - protein, 48 aa, Gossypium hirsutum
GPICKATSNVYKGLCLIDDSCDIVCKREGFHKGNCKGFLRKCVCTNPC

SEQ ID NO: 272 - protein, 48 aa, Gossypium hirsutum
KTCETKSGKFKGMCMSSTNCASVCKSEPGFDGGHCRGFRRQCVCTKPC

SEQ ID NO: 273 - protein, 48 aa, Gossypium hirsutum
TTCETQSGKFKGMCMSSTNCASVCKSEPGFDGGYCRGFRRQCFCTKPC

SEQ ID NO: 274 - protein, 48 aa, Gossypium hirsutum
RTCETKSSEYKGICLFDANCDSICKVEPSFDGGHCHGFFRRCYCTKPC

SEQ ID NO: 275 - protein, 48 aa, Gossypium hirsutum
RTCETKSSEYKGICLFDANCDSVCKVEPGFDGGHCHGFFRRCYCTKPS

SEQ ID NO: 276 - protein, 51 aa, Gossypium hirsutum
MEDRICQSRSTEYRGMCLFDVNCDNICKTEPGFTGGHCHSFFRSCYCTKPC

SEQ ID NO: 277 - protein, 48 aa, Gossypium hirsutum
RKCESPSHKFKGMCMNRDNCAIVCQTEGYEDGKCEGFRRRCICIKPCF

SEQ ID NO: 278 - protein, 55 aa, Gossypium hirsutum
GPTIVGGRKCDSPSKKFNGMCMNHDNCATVCQTESHEDGKCEGFYRRCICIKPCF

SEQ ID NO: 279 - protein, 52 aa, Gossypium hirsutum
GVVEGRICESKSHRFKGVCLSDHNCGLVCRNEGFLDGWCRGFRHRCFCTRNC

SEQ ID NO: 280 - protein, 47 aa, Gossypium hirsutum
RVCQSQSHKFHGACVSSHNCAMVCRDEGFSGGRCRGFRRRCFCTKLC

SEQ ID NO: 281 - protein, 50 aa, Gossypium hirsutum
DGDKICESPSNAFKGLCLRDDNCDIVCKTEGFPNGDCKGFLRKCVCTKPC

SEQ ID NO: 282 - protein, 48 aa, Gossypium hirsutum
KTCETQSGKFKGMCMSSTNCASVCKSEPGFDGGHCQGFRRQCVCTKPC

SEQ ID NO: 283 - protein, 48 aa, Gossypium hirsutum
KTCVAESGKFKGMCMSSTNCASVCKSEPGFDGGHCQGFRRRCLCTKPC

SEQ ID NO: 284 - protein, 48 aa, Gossypium hirsutum
RTCETKSSEYKGICLFDANCDSICKVEPGFDGGHCHGFFRRCYCTKPC

SEQ ID NO: 285 - protein, 48 aa, Gossypium hirsutum
KTCVAESGKFKGMCMSSTNCASVCKSEPDFDGGHCQGFRRRCLCTKPC

SEQ ID NO: 286 - protein, 48 aa, Gossypium hirsutum
RTCETKSSEYKGICLFDANCDSICKIESGFDGGHCHGFFRRCYCTKPC

SEQ ID NO: 287 - protein, 65 aa, Gossypium hirsutum
DEEHKEIEIGTMAEEEHKICESKSGKFKGMCFLDANCDSICKAEPGSTGGHCHGIFRICYCTKPC

SEQ ID NO: 288 - protein, 48 aa, Gossypium hirsutum
RKCESPSHKFKGMCMNRDNCATVCQTEGYEDGKCEGFRRRCICIKPCF

SEQ ID NO: 289 - protein, 52 aa, Gossypium hirsutum
GMVEGRICESKSHRFKGVCLSDHNCGLVCRNEGFLDGWCRGFRHRCFCTRNC

SEQ ID NO: 290 - protein, 47 aa, Gossypium hirsutum
RVCQSQSHKFHGACLSSHNCAMVCRNEGFSGGRCRGFRRRCFCTKLC

SEQ ID NO: 291 - protein, 50 aa, Gossypium raimondii
DGDKICESRSNAFKGLCLRDNNCDIVCKTEGFPNGGCKGFIRKCVCTKPC

SEQ ID NO: 292 - protein, 48 aa, Gossypium raimondii
GTTCKATSNIYKGLCLLDDSCDIVCKREGFHSGNCKGFLRKCVCTNPC

SEQ ID NO: 293 - protein, 50 aa, Gossypium raimondii; identical to SEQ ID NO: 281

SEQ ID NO: 294 - protein, 48 aa, Gossypium raimondii
KTCEVESGKFKGMCMSSTNCASVCKSEPGFDGGHCQGFRRRCLCTKPC

SEQ ID NO: 295 - protein, 48 aa, Gossypium raimondii; identical to SEQ ID NO: 294

SEQ ID NO: 296 - protein, 48 aa, Gossypium raimondii
RTCETKSSEYKGICLFDANCDSICKIEPGFDGGHCHGFFRRCYCTKPC

SEQ ID NO: 297 - protein, 48 aa, Gossypium raimondii; identical to SEQ ID NO: 288

SEQ ID NO: 298 - protein, 47 aa, Vitis vinifera
RVCESQSHKFEGACMGDHNCALVCRNEGFSGGKCKGLRRRCFCTKLC

SEQ ID NO: 299 - protein, 47 aa, Vitis vinifera
RTCESQSHRFKGTCVRQSNCAAVCQTEGFHGGNCRGFRRRCFCTKHC

SEQ ID NO: 300 - protein, 47 aa, Helianthus annuus
RICESPSHKFKGICLSDTNCSSVCQSEGFPWGKCIGFRRRCFCLREC

SEQ ID NO: 301 - protein, 47 aa, Helianthus annuus
RTCESQSHKFKGTCLSDTNCANVCHSERFSGGKCRGFRRRCFCTTHC

SEQ ID NO: 302 - protein, 47 aa, Helianthus annuus
RMCQSQSHMFHGTCMRDHNCALVCRNEGFSGGKCRGFRRRCFCTRLC

SEQ ID NO: 303 - protein, 47 aa, Helianthus annuus
RICESQSQEFHGSCMSNHNCGLVCRTEGFSAGICRGVRGRCFCLKTC

SEQ ID NO: 304 - protein, 47 aa, Hydrangea macrophylla
RTCESKSNRFKGPCARDSNCATVCQTEGFYGGNCKGFRRRCFCTKNC

SEQ ID NO: 305 - protein, 47 aa, Hydrangea macrophylla
RTCESKSHRFKGPCVSATNCGSVCQTEGFHGGNCRGFRRRCFCTKHC

SEQ ID NO: 306 - protein, 71 aa, Vanilla planifolia
RRCRAQSFTFKGICYSSHSCALVCKKEGYETGHCSRGFCTLLKPANAEQRAQSLQVFASVPTPALLCVSKK

SEQ ID NO: 307 - protein, 58 aa, Cicer arietinum
SEVTIRVAEGRECRDLSHKFKGFCVRDRNCANVCSTEGFTNGHCDGFRRRCFCSRPCA

SEQ ID NO: 308 - protein, 49 aa, Beta vulgaris
  source 1..49: note = subspecies Vulgaris
RVCETPSHKFQGICISSRNCASTCASEHFPGGYCRGLRRRCMCTRPCVN

SEQ ID NO: 309 - protein, 47 aa, Beta vulgaris
  source 1..47: note = subspecies Vulgaris
RTCETPSRKFRGICVSRTNCANVCKTEGFPGGRCRGFRRRCFCLEHC

SEQ ID NO: 310 - protein, 47 aa, Beta vulgaris
  source 1..47: note = subspecies Vulgaris
RICESPSYSFKGICVSRTNCANVCKTEGFPGGRCRGLRRRCFCLKHC

SEQ ID NO: 311 - protein, 47 aa, Spinacia oleracea
RTCESRSYRFKGICVSRTNCANVCKTEGFPGGRCRGVRRRCFCLKHC

SEQ ID NO: 312 - protein, 47 aa, Beta vulgaris
  source 1..47: note = subspecies Vulgaris
RTCETPSHKFKGICVSRTNCANVCKTEGFPAGRCRGFRRRCFCLTHC

SEQ ID NO: 313 - protein, 47 aa, Ipomoea triloba
RICESKSHHYKGMCLRDHNCAMVCRTEGFSGGHCIGARRRCFCTRGC

SEQ ID NO: 314 - protein, 47 aa, Lupinus albus
RTCESQSHRFKGACLSDTNCGSVCHNEGFNGGHCRGFRRRCFCTKHC

SEQ ID NO: 315 - protein, 52 aa, Lupinus albus
FLGSHRCGKVLSSRFKGKCLTDHNCDSVCQLEGYVGGDCHGVRRRCFCNKKC

SEQ ID NO: 316 - protein, 47 aa, Lupinus albus
RHCESKSHRFKGLCHSDHNCASICLVEGFTSGKCHGFRQRCFCTKVC

SEQ ID NO: 317 - protein, 49 aa, Lupinus albus
RTRVCESQSHLFKGPCSRDHNCALVCRNEAFSGGRCKGFRRRCFCTKLC

SEQ ID NO: 318 - protein, 59 aa, Lupinus albus
MVIETEGVLGRNRCDPVLSSRFKGKCFSDHNCNSVCQLEGYTDGNCHGLRQRCFCNRKC

SEQ ID NO: 319 - protein, 59 aa, Lupinus albus
SDNNVGLKRAEGRVCQSKSHRFKGPCLSYSNCGHVCRNEGFSGGRCRGLRRRCFCTKNC

SEQ ID NO: 320 - protein, 59 aa, Lupinus albus
EMGPTMMEEARTCKSKSQRYKGSCYSDNNCAIICEGEGFTGGKCHGFFCKCWCKRPCLD

SEQ ID NO: 321 - protein, 69 aa, Lupinus albus
KECSTKSDRFRGTCFRSSNCAAVCNSENFPSGTCRGFPGTCYCTSNCAINAPITNPPPEVVEPPPEVVE

SEQ ID NO: 322 - protein, 48 aa, Lupinus albus
KTCESRSHKLKGFCYSSKRCAHVCTSHHNFIGGHCRGFIRRCHCTKPC

SEQ ID NO: 323 - protein, 48 aa, Lupinus albus
NNCKYESNKFHGVCLSDTNCESVCQTEGFPFGKCDGVLPRTCVCKKPC

SEQ ID NO: 324 - protein, 47 aa, Lotus japonicus
RHCESKSHRFHGLCLSHHNCASVCHLEGFTGGKCRGFRKRCFCKKRC

SEQ ID NO: 325 - protein, 48 aa, Lotus japonicus
RVCQSQSHRFRGGCWGDHNCALVCRNEGFSGGRCRGFRRRCFCTRLCN

SEQ ID NO: 326 - protein, 47 aa, Lotus japonicus
RVCETKSHTFHHSCVSGHNCAQVCKTEGFTGGKCRGFRRRCFCTKRC

SEQ ID NO: 327 - protein, 47 aa, Lotus japonicus
RDCESQSHRFKGPCVSDTNCASVCHGEKFSGGHCRGFRRRCFCTKHC

SEQ ID NO: 328 - protein, 55 aa, Lactuca sativa
MGVETKSCRYSSKTYKGLCFYSRNCDIICKAEGAPGGGHCQILTLRCFCNVCDPD

SEQ ID NO: 329 - protein, 47 aa, Manihot esculenta
RICESQSHRFKGVCVRKSNCAAICQTEGFHGGHCRGLRRRCFCTKHC

SEQ ID NO: 330 - protein, 47 aa, Manihot esculenta
RVCESKSHHFRGLCTIAHNCAMVCRNEGFSGGKCRGFRRRCFCTRLC

SEQ ID NO: 331 - protein, 47 aa, Manihot esculenta
RVCESKSHHFRGVCTIAHNCAIVCRNEGFSGGKCKGFRRRCFCTRLC

SEQ ID NO: 332 - protein, 47 aa, Manihot esculenta
RKCESQSHKFKGICLSDTNCASVCKTEGFTGGDCKGARRRCFCTRQC

SEQ ID NO: 333 - protein, 47 aa, Manihot esculenta
RKCESQSHRFKGMCTRHSNCAAICQTEGFHAGHCRGLRRRCFCTKHC

SEQ ID NO: 334 - protein, 47 aa, Malus domestica
RVCQSQSHGFHGTCLRHHNCALVCRNEGFSGGRCRGLRRRCFCTRLC

SEQ ID NO: 335 - protein, 57 aa, Malus domestica
RTESSKAVEGKICEFPSTLFKGLCFSSNNCKHTCRKEQFTRGKCSLLTRTCMCTKKC

SEQ ID NO: 336 - protein, 47 aa, Malus domestica
RTCESQSNRFKGTCVSKSNCAAVCQTEGFPGGNCRGLRRRCFCTKHC

SEQ ID NO: 337 - protein, 47 aa, Malus domestica
RTCESHSRKYKGTCLGKSNCASVCQTEGFHGGHCRGFRRRCFCTKHC

SEQ ID NO: 338 - protein, 63 aa, Malus domestica
RVCEVPSKTFRGLCFSDTHCIVKCHSEGFGYGKCSHVLRQCRCLKACGRLAESETTHGTETSP

SEQ ID NO: 339 - protein, 57 aa, Malus domestica
RTEPSKAVEGKICEVPSTLFKGLCISSNNCKHTCRKEQFTRGHCSVFTRACVCTKKC

SEQ ID NO: 340 - protein, 57 aa, Malus domestica
RVESSKAVEGKICEVPCTLFKGLCFSSNNCKHTCRKEQFTRGHCSVFTRACVCTKKC

SEQ ID NO: 341 - protein, 57 aa, Malus domestica
RTESNKAVEGKICEYPSTLFKGLCFSSNNCKHTCRKEQFTRGHCSVFTRACVCTKKC

SEQ ID NO: 342 - protein, 60 aa, Malus domestica
KTNENSSKNEKSSKICESPSHKYRGICFRSSNCATTCKKEGFMGGKCAGFRMRCVCTKKC

SEQ ID NO: 343 - protein, 60 aa, Malus domestica
KTKEESSKNEKSSKICESPSHKYRGICFRSSNCATTCKKEGFMGGKCAGFRMRCVCTKKC

SEQ ID NO: 344 - protein, 59 aa, Malus domestica
KTESSKAVEGKICEVPSTLFKGLCISSNNCKHTCRKEQFTRGKCIGDGFTRKCVCTKKC

SEQ ID NO: 345 - protein, 57 aa, Malus domestica
RSKSSKEVQERTCEAASGKFKGLCFSSTNCKNTCKREQFTGGKCRGFRRRCMCNKKC

SEQ ID NO: 346 - protein, 57 aa, Malus domestica
RSKSDKVAKERTCEAASGKFKGLCFSSTNCKNTCKVEKFTGGQCQGFRRRCMCNKKC

SEQ ID NO: 347 - protein, 57 aa, Malus domestica
RSKSGKAVKERTCEATSGKFKGMCFSSTNCKNTCKTEKFIGGECHGFRRRCICNKKC

SEQ ID NO: 348 - protein, 57 aa, Malus domestica
RTESSKAIEGKICEVPSTLFKGLCFSSNNCKHTCRKEQFTRGKCSGLTRTCVCTKKC

SEQ ID NO: 349 - protein, 57 aa, Malus domestica
RTESSKAVEGKICEVPSTLFKGLCFSSNNCKHTCRKEQFTRGKCSGLTRTCMCTKKC

SEQ ID NO: 350 - protein, 57 aa, Malus domestica
MTESNKAVEGKICEVPSTLFKGLCFSSNNCKHTCRKEQFTRGKCSGLTRTCMCTKKC

SEQ ID NO: 351 - protein, 60 aa, Malus domestica
VEARSKSSKVVEERTCEAASGKFKGLCFSSNNCKHTCKREQFTGGKCRGFRRRCMCNKKC

SEQ ID NO: 352 - protein, 47 aa, Malus domestica
RTCDSQSHRFKGSCVSKSNCATVCQTEGFRGGHCRGFRRRCFCTKHC

SEQ ID NO: 353 - protein, 60 aa, Malus domestica
KTKEKSSKNEKSSKTCESPSHKYHGICFRSSNCATTCKKEGFMGGKCTGFWMRCVCTKKC

SEQ ID NO: 354 - protein, 60 aa, Malus domestica
KTKEKSSKNEKSSKICESPSHKFHGICFRSSNCATTCKKEGFMGGKCTGFRMRCVCTKKC

SEQ ID NO: 355 - protein, 61 aa, Malus domestica
KIRHMSKDENSLTCESRSHKYHGPCFRFIFSSNCAVTCKGEGFTGGKCKGFHMRCICTKQC

SEQ ID NO: 356 - protein, 59 aa, Malus domestica
KTKGKPSKNVKLSKTCESPSQKYPGICRSSYCAAICKQQGLMGGKCTGFRRKCVCTKKC

SEQ ID NO: 357 - protein, 57 aa, Malus domestica; identical to SEQ ID NO: 17

SEQ ID NO: 358 - protein, 60 aa, Malus domestica
RSGMTSKGAAEKKKCDSHPSSKFKGVCFFTNNCVDTCKLEGSSGGQCRGFRRRCICTKQC

SEQ ID NO: 359 - protein, 57 aa, Malus domestica
KSKSSKEVEKRTCEAASGKFKGMCFSSNNCANTCAREKFDGGKCKGFRRRCMCTKKC

SEQ ID NO: 360 - protein, 47 aa, Malus domestica
RTCESQSNRFKGSCVSKSNCAAVCQTEGFPGGNCRGLRRRCFCTKHC

SEQ ID NO: 361 - protein, 47 aa, Malus domestica
RTCESQSRKFKGTCLSKTNCASVCQTEGFPGGNCRGFRRRCFCTKHC

SEQ ID NO: 362 - protein, 57 aa, Medicago truncatula
QPTHVEEPEARTCDSQSHSFKGVCWIKHNCANVCKTEGFTGGHCHGFRRRCFCSKPC

SEQ ID NO: 363 - protein, 76 aa, Medicago truncatula
EEFNFAPFTPLHFRVESCFTRSRRYNGICLSDESCFKACSLEGYACGRCEKRRPYLGDHCCCKKSCQLKLVLAVLC

SEQ ID NO: 364 - protein, 50 aa, Medicago truncatula
RKHCREKSRLFEELCFNSEDCANTCRYEGFHLGGKCWGLFRTCYCKKKCR

SEQ ID NO: 365 - protein, 47 aa, Medicago truncatula
RHCESKSHRFKGMCMSDHNCASVCHVEGFPGGNCRGFRRRCFCKKRC

SEQ ID NO: 366 - protein, 47 aa, Medicago truncatula
RTCESQSHKFKGPCASDHNCASVCQTERFSGGHCRGFRRRCFCTTHC

SEQ ID NO: 367 - protein, 47 aa, Medicago truncatula
RTCETPSNSFKGACFSDTNCASVCQTEGFPGGHCKGFRQRCFCTKPC

SEQ ID NO: 368 - protein, 47 aa, Medicago truncatula
RTCETPSNNFKGLCVSDTNCASVCQTEGFPGGHCEGFRQRCFCTKPC

SEQ ID NO: 369 - protein, 50 aa, Medicago truncatula
RKCLSQSHSFKGLCLSDQNCATVCLTEGFTDGRCRGFRQRCFCSKPCLKV

SEQ ID NO: 370 - protein, 47 aa, Medicago truncatula
RRCESKSHKFKGPCSRDSNCASVCRGEGFTGGDCRGFRRRCFCTRNC

SEQ ID NO: 371 - protein, 47 aa, Medicago truncatula
RRCESQSHKFKGPCVSDSNCGSVCRGEGFIGGDCRGVRHRCFCTRNC

SEQ ID NO: 372 - protein, 48 aa, Cucumis melo
RVCESPSHNFKGLCFSDTNCGNICKTEGFSGGLCRGFRRRCFCTKHCV

SEQ ID NO: 373 - protein, 47 aa, Cucumis melo
RMCQSQSHHFHGTCIRDHNCALVCRTEGFSGGECVGFRRRCFCTHRC

SEQ ID NO: 374 - protein, 58 aa, Mimulus guttatus
VKEVGTVEDEKKVCKYKSKKFWGVCLSDIRCTDVCKREGFQGGDCQGIRRRCLCYNRC

SEQ ID NO: 375 - protein, 48 aa, Mimulus guttatus
TRTCEAKSHRFKGICARKANCAAVCQTEGFHGGHCRGFRRRCFCTKHC

SEQ ID NO: 376 - protein, 47 aa, Mimulus guttatus
RVCESKSASYKGVCVLDVTCKLACLNEGFADGDCEGVRRRCMCRRPC

SEQ ID NO: 377 - protein, 39 aa, Mimulus guttatus
FKGICVLDETCRLACLNEGFSDGDCEGLRRRCLCRKPCK

SEQ ID NO: 378 - protein, 72 aa, Mimulus guttatus
ALCETKSSIFTGACFSDRNCETICEKEGFLNGRCKLWKCICSKDCGIGGGGGVVVPPGGGGDVPPGGGDDVP

SEQ ID NO: 379 - protein, 48 aa, Mimulus guttatus
RMCETQSHGFKGQCWSEHNCRLVCAHEGFRGGKCRGFRKRCFCTRPCH

SEQ ID NO: 380 - protein, 47 aa, Mimulus guttatus
RTCLSQSQRFRGPCVSDTNCRLVCHNEGFSGGDCQGFRRRCFCTKHC

SEQ ID NO: 381 - protein, 64 aa, Mimulus guttatus
QSNVDGEIADYLKIEEKKRCNYESKKFWGICLSDSKCGEVCTKEGFEGGDCKGLRRRCYCFNNC

SEQ ID NO: 382 - protein, 71 aa, Mimulus guttatus
SLCETPSRYFNGICFSRRNCGAICEKEGFSSGRCKAFKCVCGKDCNAAGGGGGGGGGDPGLGPPVEGPPDQ

SEQ ID NO: 383 - protein, 47 aa, Mimulus guttatus
RTCESQSHGFKGRCLSDHNCGMVCKNEGFSNGWCRGFRQRCFCTRAC

SEQ ID NO: 384 - protein, 47 aa, Mimulus guttatus
RTCTSKSSNFKGLCFSSHNCKAVCNGENFPDGKCHGFKRRCFCQKPC

SEQ ID NO: 385 - protein, 47 aa, Mimulus guttatus
RTCESQSFNFRGPCVIDHNCKAVCESEGFTDGDCQGFRRRCFCRKPC

SEQ ID NO: 386 - protein, 62 aa, Handroanthus impetiginosus
ALCEKPSRLYNGSCTEERLCIEICQKEAYPSGRCDLLKCMCINKCPPVGAGQGAGSDGQRGI

SEQ ID NO: 387 - protein, 47 aa, Nicotiana tabacum
RTCESQSHRFKGPCSRDSNCATVCLTEGFSGGDCRGFRRRCFCTRPC

SEQ ID NO: 388 - protein, 47 aa, Nicotiana tabacum
RHCESQSQRFRGLCVREKNCAAVCETEGFSGGDCRGLRRRCFCTRPC

SEQ ID NO: 389 - protein, 47 aa, Nicotiana tabacum
RVCMSPSHGYHGPCWHDHNCALVCRNEGFSGGDCVGIQRKCYCTKLC

SEQ ID NO: 390 - protein, 47 aa, Nicotiana tabacum
RTCESQSHRFHGTCVRGSNCASVCQTEGFIGGNCRGLRRRCFCTRNC

SEQ ID NO: 391 - protein, 58 aa, Nicotiana tabacum
TEMGPMTIAEARRCESKSQRFRGPCVREKNCAAVCETEGFSGGDCRGLRRRCFCTRPC

SEQ ID NO: 392 - protein, 48 aa, Nelumbo nucifera
RTCESQSHRFKGICVRKSNCAAVCQTEGFHGGHCRGFRRRCFCTKHCA

SEQ ID NO: 393 - protein, 49 aa, Nelumbo nucifera
KMCKTPSRQFRGLCLIGRNCDNVCRGEGFPDGDCDGFRRRCICSRPCRP

SEQ ID NO: 394 - protein, 100 aa, Nelumbo nucifera
RMCDTPSQKFVGLCLIDRNCENVCKTEGFPAGHCEGLRRRCFCSEPCPPNNGMERKFLCGVLLFLTLLYASLGEEMVVNEGPADGGRVCSTPSLNYRGPC

SEQ ID NO: 395 - protein, 49 aa, Nelumbo nucifera
RMCDTPSRLFRGLCLINRNCDNTCRVEGFPDGHCKGLRRRCFCSRPCRP

SEQ ID NO: 396 - protein, 49 aa, Olea europaea
RICESLSHRFKGPCVRNANCAAVCQTEGFPGGHCRGFRRRCFCTKPCSD

SEQ ID NO: 397 - protein, 47 aa, Olea europaea
RICESLSHRFKGPCVRRGNCAAVCQTEGFPGGLCRGFRRRCFCTKHC

SEQ ID NO: 398 - protein, 70 aa, Oryza sativa
IICKARSKMYRGKCRGNRNCAMICVHEEYTGGYCSKGVFSKCMCTKRCGGGGGGGGGGGGGGGDEPPLRE

SEQ ID NO: 399 - protein, 70 aa, Oryza sativa
ICHDHSQTFKGMCFHTSNCIACCTNEGYTGGYCKPFTYRCMCTKDCGGDSPPDDPPPAMPTSPAATTTVA

SEQ ID NO: 400 - protein, 59 aa, Oryza sativa
RLCEKPSSHFKGLCLRSQNCDNECMLEGYMDGKCKYLTRRCICSMECATMSNVGLTPTK

SEQ ID NO: 401 - protein, 49 aa, Oryza sativa
RHCLSQSHRFKGMCVSSNNCANVCRTESFPDGECKSHGLERKCFCKKVC

SEQ ID NO: 402 - protein, 48 aa, Oryza sativa
RTCESQSHRFKGPCARKANCASVCNTEGFPDGYCHGVRRRCMCTKPCP

SEQ ID NO: 403 - protein, 49 aa, Oryza sativa
RHCVSQSHRFVGACMRKSNCEHVCMTEGFPWGECRFHGIERKCFCKKRC

SEQ ID NO: 404 - protein, 47 aa, Petunia axillaris
RTCESQSHRFKGPCVSKSNCASVCQTEGFHGGHCRGLRRRCFCTKHC

SEQ ID NO: 405 - protein, 49 aa, Petunia axillaris
RYRVCLSPSHRYKGMCLRDHNCALVCRNEGFSGGDCIGFRRRCFCTKLC

SEQ ID NO: 406 - protein, 49 aa, Petunia axillaris
EARICESQSHRFHGTCVRESNCASVCQTEGFIGGNCRGFRRRCFCTRNC

SEQ ID NO: 407 - protein, 47 aa, Petunia axillaris
RTCETPSQRFKGPCVSDTNCANVCQTEGFPGGDCQGLRRRCFCTKPC

SEQ ID NO: 408 - protein, 47 aa, Petunia axillaris
RTCETLSHRFRGPCVREKNCAAVCETEGFSGGDCRGLRRRCFCTRPC

SEQ ID NO: 409 - protein, 47 aa, Petunia axillaris
RVCMSPSHGYRGPCIRDHNCALVCRDEGFSGGDCVGIQRKCYCTKLC

SEQ ID NO: 410 - protein, 50 aa, Petunia axillaris
RTCEHRSQHYYGACFANNSNMNNNCDSMCKCDGFDGGFCRLTNCFCHVKC

SEQ ID NO: 411 - protein, 47 aa, Petunia axillaris
KLCQYKSKTFHGVCVTGNTCNQKCQSEAFDGGRCQGVRHRCYCYRTC

SEQ ID NO: 412 - protein, 51 aa, Petunia axillaris
RVCRTNSKRYSGPCFNNNNNRNQNCDFICRRCEGFEGGHCSSRTCVCTKSC

SEQ ID NO: 413 - protein, 47 aa, Punica granatum
RTCESQSHKFRGTCVSNTNCGSVCQTEGFHGGHCRGFRRRCFCTKQC

SEQ ID NO: 414 - protein, 47 aa, Punica granatum
RICESKSHRFRGVCMSNSNCASVCHNEGFPGGRCRGVRKRCFCTKVC

SEQ ID NO: 415 - protein, 47 aa, Solanum tuberosum
RTCESQSHRFKGPCVSEKNCASVCETEGFSGGDCRGLRRRCFCTRPC

SEQ ID NO: 416 - protein, 47 aa, Solanum tuberosum
RNCESLSHRFKGPCTRDSNCASVCETERFSGGNCHGFRRRCFCTKPC

SEQ ID NO: 417 - protein, 47 aa, Solanum tuberosum
RTCESQSRRFKGLCFSKSNCGSVCHTEGFSGGHCRGFRRRCFCTRHC

SEQ ID NO: 418 - protein, 47 aa, Solanum tuberosum
RTCESQSHKYKGPCVRKSNCGAVCQTEGFTGGHCRGVRRRCFCTKHC

SEQ ID NO: 419 - protein, 47 aa, Solanum tuberosum
RTCESQSHSFKGPCVGDTNCASVCQTEGFIGGDCRGLRRQCFCTRNC

SEQ ID NO: 420 - protein, 47 aa, Solanum tuberosum
RTCESQSNRFKGTCVRDSNCATVCQTEGFIGGNCRGFRRRCFCTRNC

SEQ ID NO: 421 - protein, 49 aa, Solanum tuberosum
RVCISQSHRYKGPCLRDHNCALVCRNEYFSGGDCIGFGFHRKCYCTKFC

SEQ ID NO: 422 - protein, 46 aa, Phaseolus vulgaris
KTCFTKKEGWGRCIADFTCARSCRNVGYIGGKCQGLTRRCFCVLNC

SEQ ID NO: 423 - protein, 50 aa, Phaseolus vulgaris
AEGKICRTKSRKYTGACYSNDICANYCKGDGFDDGDCGGLSHICFCTKNC

SEQ ID NO: 424 - protein, 47 aa, Phaseolus vulgaris
RLCESPSHSFKGLCFNDRNCANVCLTEGFTGGKCEGARHRCFCTIIC

SEQ ID NO: 425 - protein, 47 aa, Phaseolus vulgaris
RLCWSESQAFKGLCFSNDNCATVCLTEGFTGGKCEGLRRRCFCSKNC

SEQ ID NO: 426 - protein, 47 aa, Phaseolus vulgaris
RGCESRSRHLRGKCESDENCASACRTERFSGGHCRGFRCRCFCDKRS

SEQ ID NO: 427 - protein, 47 aa, Phaseolus vulgaris
RTCESQSHRFKGPCVSDTNCASVCRTERFSGGHCRGFRRRCFCTKHC

SEQ ID NO: 428 - protein, 47 aa, Phaseolus vulgaris
RVCESQSHGFKGACTGDHNCALVCRNEGFSGGNCRGFRRRCFCTKIC

SEQ ID NO: 429 - protein, 47 aa, Phaseolus vulgaris
RHCESKSHRFKGMCLSDRNCASVCHLEGFTGGNCRGFRRRCFCTRHC

SEQ ID NO: 430 - protein, 47 aa, Populus trichocarpa
RVCLSQSHHYKGPCLRGHNCAMVCKTEGFAGGECKGFISRCFCAKLC

SEQ ID NO: 431 - protein, 47 aa, Populus trichocarpa
RVCLSQSHSFKGPCVRGHNCASVCKTEGFPGGECKGFRRRCFCAKPC

SEQ ID NO: 432 - protein, 47 aa, Populus trichocarpa; identical to SEQ ID NO: 431

SEQ ID NO: 433 - protein, 47 aa, Populus trichocarpa
RVCQSQSHYFKGPCARDHNCAWVCRNEGFSGGRCKGFRRRCFCTKLC

SEQ ID NO: 434 - protein, 47 aa, Populus trichocarpa; identical to SEQ ID NO: 433

SEQ ID NO: 435 - protein, 47 aa, Prunus persica
RVCQSQSHGFRGPCIRHHNCALVCRNEGFSGGRCRGFRRRCFCTRLC

SEQ ID NO: 436 - protein, 47 aa, Prunus persica
RTCESQSNRFKGTCVSTSNCASVCQTEGFPGGHCRGFRRRCFCTKHC

SEQ ID NO: 437 - protein, 57 aa, Prunus persica
RDAKKVEPKKKTCDSQSQKFKGMCFLTSSCVQSCKSEGFTGGQCRGFRRRCFCSKNC

SEQ ID NO: 438 - protein, 57 aa, Prunus persica
RSAKKVEPKKRTCESQSQKFKGICFLSSNCATSCTTEGFNGGQCRGFRRRCFCSKAC

SEQ ID NO: 439 - protein, 57 aa, Prunus persica
KICEVTSKTFIGLCIKDRNCMVRCHSEGYGYGKCSHILRKCRCLKPCVAEETKENLP

SEQ ID NO: 440 - protein, 55 aa, Prunus persica
KVATKEESRTCESLSTKFKGFCLRSSRCAEACQKEGFMGGKCPGFRLRCTCTKKC

SEQ ID NO: 441 - protein, 55 aa, Prunus persica; identical to SEQ ID NO: 440

SEQ ID NO: 442 - protein, 55 aa, Prunus persica
KVETKETSRTCESLSTKFKGPCIRSSNCANICEEEGFKGGKCVGFRLRCTCTKNC

SEQ ID NO: 443 - protein, 55 aa, Prunus persica; identical to SEQ ID NO: 442

SEQ ID NO: 444 - protein, 55 aa, Prunus persica; identical to SEQ ID NO: 442

SEQ ID NO: 445 - protein, 55 aa, Prunus persica; identical to SEQ ID NO: 442

SEQ ID NO: 446 - protein, 55 aa, Prunus persica
KVETKETSRTCESLSTKFKGPCFRSSNCANICEKEGFKGGKCVGFRLRCTCTKNC

SEQ ID NO: 447 - protein, 55 aa, Prunus persica
KAATKENSRTCESLSTKFKGLCFRSSNCADICKKEGFLEGKCAGFRLRCTCTKKC

SEQ ID NO: 448 - protein, 47 aa, Pisum sativum
RDCESKSHKFKGTCLSDTNCASVCQTERFTGGHCRGFRHRCYCTTHC

SEQ ID NO: 449 - protein, 47 aa, Pisum sativum
RDCESPSQKFKGLCFSDTNCASVCQTEGFPGGKCEGLRHRCFCTKPC

SEQ ID NO: 450 - protein, 47 aa, Pisum sativum
RDCESPSQKFKGVCLSDRNCASVCQTEGFTGGDCKGLRQRCFCTKPC

SEQ ID NO: 451 - protein, 47 aa, Pisum sativum
RDCESPSQIFKGLCFTDTNCASVCHTEGFPGGKCKGLPKRCICTKPC

SEQ ID NO: 452 - protein, 46 aa, Pisum sativum
AGTCPSPSTKFKGVCVSDFDCMITCQMEGLSEGKCQSLKCICAKPC

SEQ ID NO: 453 - protein, 39 aa, Pisum sativum
SECNDEECEWSCIDKGFLDGVCSPSSNQCFCVIHRVIIR

SEQ ID NO: 454 - protein, 50 aa, Pisum sativum
NSCQSKSSQFKGICLSNKKCARVCHLEGFLEGGKCKGKLLKHCSCKKSIC

SEQ ID NO: 455 - protein, 45 aa, Pisum sativum
RGCKVKSTKFRGPCSFDKNCATVCRGEGYRDGDCHGLRRRCMCLC

SEQ ID NO: 456 - protein, 47 aa, Pisum sativum
RKCVSQSHRFHGPCLRDSNCATVCLGEGFTGGDCHGFRRRCFCSRFC

SEQ ID NO: 457 - protein, 56 aa, Pisum sativum
RHCESPSHRFKGICVRKSNCAAICQTEGFSSGKCRGFRSRCVCIKHCHHHEEEVQN

SEQ ID NO: 458 - protein, 57 aa, Pisum sativum
RLCESASHRFKGICVRKSNCGAVCQTEGFSGGKCRGLRARCMCTKHCHQEEEAEVKN

SEQ ID NO: 459 - protein, 57 aa, Pisum sativum
MRVDGARTNRMCDSKSHKFRGLCVSDTNCRNVCQTERFSDGHCRGARRRCYCQNPCI

SEQ ID NO: 460 - protein, 57 aa, Pisum sativum; identical to SEQ ID NO: 458

SEQ ID NO: 461 - protein, 47 aa, Quercus lobata
RTCESQSHRFKGPCVRKSNCASVCQTEGFHGGQCRGFRRRCFCTKHC

SEQ ID NO: 462 - protein, 47 aa, Quercus lobata
RVCESQSHKFKGACMSNHNCALVCRNEGFSGGRCRGFRHRCFCTRIC

SEQ ID NO: 463 - protein, 50 aa, Rosa chinensis
ENLRTCDSPSGKFKGPCIISSNCALTCQKEGFAGGHCRDFLRKCYCAKPC

SEQ ID NO: 464 - protein, 47 aa, Rosa chinensis
RTCETLSHKFKGTCLRESNCASVCQTEGFSGGDCRGLRRRCFCTKHC

SEQ ID NO: 465 - protein, 59 aa, Rosa chinensis
GIGPNVMVAEARTCESQSLKYEGMCLRESHCASVCQTEGYSGGDCHGLYSICVCTKDCQ

SEQ ID NO: 466 - protein, 58 aa, Rosa chinensis
GIGPNVTVAEARKCESQSHKFAGICLIESNCASICKTEGYSGGNCGGFRRRCFCTKDC

SEQ ID NO: 467 - protein, 47 aa, Rosa chinensis
RKCESQSHKFEGICLKQSNCASICKTEGYSGGNCRGFRRRCFCTKDC

SEQ ID NO: 468 - protein, 58 aa, Rosa chinensis
GIGPNVMVAEARKCESQSHKFAGICLIESNCASICKTEGYSGGNCGGFRRRCFCTKDC

SEQ ID NO: 469 - protein, 47 aa, Rosa chinensis
RVCEVPSKTFSGLCFSDTHCIVRCHSEGYGYGKCSHILRRCRCLKAC

SEQ ID NO: 470 - protein, 48 aa, Rosa chinensis
KICEVPSKTFKGLCVKDRNCVVRCHSEGYGNGKCSHVRRQCMCLKICD

SEQ ID NO: 471 - protein, 47 aa, Rosa chinensis
KICEYPSRTFKGLCVKDKNCVVRCRSEGYNYGKCSHVRRRCRCFKPC

SEQ ID NO: 472 - protein, 47 aa, Rosa chinensis; identical to SEQ ID NO: 242

SEQ ID NO: 473 - protein, 47 aa, Rosa chinensis
RVCESRSHGFHGTCIHHNNCALVCRNEGFPGGRCRGFRRRCFCTRNC

SEQ ID NO: 474 - protein, 47 aa, Salvia bowleyana
RTCESKSHRFKGICVRKANCAAVCQTEGFHSGHCRGFRRRCYCTRHC

SEQ ID NO: 475 - protein, 42 aa, Salvia bowleyana
RTCESESSSFKGLCLRNADCYIVCQTEGFSGGKCSCFCTKPC

SEQ ID NO: 476 - protein, 47 aa, Salvia bowleyana
RTCESESSTFKGLCLKNADCYIVCRTEGFSGGKCSSFLRRCFCTKPC

SEQ ID NO: 477 - protein, 74 aa, Salvia bowleyana
VVCSAASRLFSGLCFSDSNCSSICEKEGFLTGVCKGLQCICQKDCGGGDGGDPGQGGADQSALINRKLLDQLLV

SEQ ID NO: 478 - protein, 56 aa, Salvia bowleyana
AAASVVEEKRSCQSQSHKFKGKCFSDKNCGSVCQTEGFPSGKCHGLRQRCFCTRPC

SEQ ID NO: 479 - protein, 56 aa, Salvia bowleyana
AMCSAPSKLFRGVCLLRSNCSSICATEGFLYGLCKDLKCVCYKDCNVVGSGGDAQH

SEQ ID NO: 480 - protein, 74 aa, Salvia bowleyana
VMCSAASRLFSGLCFSDNNCSAICEKEGFLSGVCKGLKCVCQKDCNGGGGGGGNGGGDGGDQDQGPPAQGGGDQ

SEQ ID NO: 481 - protein, 74 aa, Salvia bowleyana
VMCSAASRLFSGLCFSDNNCSAICEKEGFLSGVCKGLKCVCQKDCNGGGGGNGGGDGGDQDQGPPAQGGGDQSP

SEQ ID NO: 482 - protein, 73 aa, Salvia bowleyana
AMCSAPSRLFRGLCFRRSNCTSICEKEGFLYGLCKGLKCVCYKDCNAAGGGGNGGDPGQGPPDQGGDDQPPPL

SEQ ID NO: 483 - protein, 56 aa, Salvia bowleyana; identical to SEQ ID NO: 478

SEQ ID NO: 484 - protein, 61 aa, Salvia bowleyana
AMCSAPSKLFRGVCLLRSNCSSICATEGFLYGLCKDLKCVCYKDCNVVGSGSGGSGGDAQH

SEQ ID NO: 485 - protein, 48 aa, Simmondsia chinensis
RICQSQSHLFRGPCVRNHNCASVCQNEGFSDGSCRGFRRRCFCAKFCP

SEQ ID NO: 486 - protein, 42 aa, Sechium edule
QSHGFHGACLSDHNCAVVCRNEGFSGGDCKGFRRRCFCTRHC

SEQ ID NO: 487 - protein, 48 aa, Sechium edule
RTCESPSRHFKGVCFSRNNCGHVCKTEGFHGGHCRGFRRRCFCTKHCL

SEQ ID NO: 488 - protein, 48 aa, Sechium edule
RTCESQSHHFKGLCFSKNNCGHVCKTEGFHGGHCRGFRRRCFCTKHCA

SEQ ID NO: 489 - protein, 50 aa, Sequoiadendron giganteum
RMCKSQSHTFKGYCVSDSNCKNVCRTEKFPTGGCDFHVASRKCYCYKPCA

SEQ ID NO: 490 - protein, 47 aa, Solanum lycopersicum
RTCESQSHRYKGPCVRKNNCANVCKTEGFSGGHCRGFRRRCFCAKHC

SEQ ID NO: 491 - protein, 47 aa, Solanum lycopersicum
RTCESQSHHFKGNCLSDTNCGSVCRTEGFTGGNCRGFRRRCFCTRNC

SEQ ID NO: 492 - protein, 50 aa, Solanum lycopersicum
AEARTCESQSNSFKGTCVRDSNCATVCQTEGFIGGNCRGFRRRCFCTRNC

SEQ ID NO: 493 - protein, 47 aa, Solanum lycopersicum
RTCEPQSHSFKGPCSRDSNCATVCQTEGFIGGDCRGLRRQCFCTTEC

SEQ ID NO: 494 - protein, 47 aa, Solanum lycopersicum
RTCESQSHRFKGPCVSEKNCASVCETEGFSGGDCRGFRRRCFCTRPC

SEQ ID NO: 495 - protein, 47 aa, Solanum lycopersicum
RVCMSPSHSYHGPCWHDHNCAIVCRNEGFSGGNCVGIQLKCYCTKLC

SEQ ID NO: 496 - protein, 47 aa, Solanum lycopersicum
RVCISQSHRYRGPCVLDHNCAIICRNEGFSGGDCIGWRRRCFCTKLC

SEQ ID NO: 497 - protein, 47 aa, Solanum pennellii
RTCETQSHKYKGPCVRKSNCANVCKTEGFRGGHCRGFRRRCFCTKNC

SEQ ID NO: 498 - protein, 55 aa, Solanum pennellii
GIRKTDAKLCQYHSETFHGVCVTGNNCNRHCQRENFEGGRCHGIRHYRCVCYRTC

SEQ ID NO: 499 - protein, 47 aa, Solanum pennellii
RHCESLSHRFKGPCASDKNCASICETERFSGGNCRGFRRRCFCTKPC

SEQ ID NO: 500 - protein, 47 aa, Solanum pennellii; identical to SEQ ID NO: 494

SEQ ID NO: 501 - protein, 47 aa, Solanum pennellii
RTCESQSHSFKGPCSRDSNCATVCQTEGFIGGDCRGLRRQCFCTTDC

SEQ ID NO: 502 - protein, 47 aa, Solanum pennellii
RTCESQSNSFKGTCVRDSNCATVCQTEGFIGGNCRGFRRRCFCTRNC

SEQ ID NO: 503 - protein, 47 aa, Solanum pennellii; identical to SEQ ID NO: 491

SEQ ID NO: 504 - protein, 47 aa, Solanum pennellii; identical to SEQ ID NO: 495

SEQ ID NO: 505 - protein, 47 aa, Solanum pennellii; identical to SEQ ID NO: 496

SEQ ID NO: 506 - protein, 47 aa, Schrenkiella parvula
RTCESKSHRFRGVCVSDGNCGNVCHNEGFGGGKCRGFRRRCFCTRHC

SEQ ID NO: 507 - protein, 47 aa, Schrenkiella parvula
RTCESQSHRFKGKCLSDGNCGNVCHNEGFGGGKCRGFRRRCFCTRNC

SEQ ID NO: 508 - protein, 45 aa, Schrenkiella parvula
KTCESPSGKYHGVCLNAKNCASVCPSEGFTGGHCKSLRCYCIKTC

SEQ ID NO: 509 - protein, 63 aa, Schrenkiella parvula
TEMGPADVEPRICETLSKVFKGMCWNQNNCAIMCREFEHFHSGHCRGFLRRCYCTKRCDGGPS

SEQ ID NO: 510 - protein, 52 aa, Tarenaya hassleriana
ERGEGRMCQSKSHHFHRTCVSNHNCAMVCRTEGFSGGHCVGFRRRCFCTRLC

SEQ ID NO: 511 - protein, 47 aa, Tarenaya hassleriana
RTCESPSHRFKGACLSETNCANVCHNEGFIGGNCRGLRRRCFCTKHC

SEQ ID NO: 512 - protein, 47 aa, Theobroma cacao
RTCQSQSHRFKGMCMRKSNCAAVCQTEGFHSGHCRGFHRRCFCTKHC

SEQ ID NO: 513 - protein, 47 aa, Theobroma cacao
RTCESKSHAFKGPCLSDHNCGLVCRNEGFPDGWCRGIRRRCFCTRRC

SEQ ID NO: 514 - protein, 47 aa, Theobroma cacao
RLCESKSHGFKGACLSDHNCGMVCSNEGFSGGRCRGFRHRCFCTRRC

SEQ ID NO: 515 - protein, 47 aa, Theobroma cacao
RVCESKSHKFKGPCLGDHNCALVCRNEGFSGGRCRGFRHRCFCTKLC

SEQ ID NO: 516 - protein, 47 aa, Thlaspi arvense
RTCESKSHRFKGACLSETNCKNVCHNEGFRGGNCRGLRRRCFCTRNC

SEQ ID NO: 517 - protein, 47 aa, Eutrema salsugineum
RKCESKSHRFKGKCLSETNCKNVCHNEGFTGGNCRGFRRRCFCTRNC

SEQ ID NO: 518 - protein, 46 aa, Eutrema salsugineum
RTCESASGKFQGVCLNSKSCASVCQSNEGFSGGHCNSLRCYCTKTC

SEQ ID NO: 519 - protein, 47 aa, Eutrema salsugineum
RMCQSKSHHFKHMCWSDHNCAIICRNEGFSGGRCRGFHRRCYCTRLC

SEQ ID NO: 520 - protein, 50 aa, Eutrema salsugineum
QQICKARSINFTALCLRWSKCKQACLTENFPDGRCIARRCFCMTPCHFLT

SEQ ID NO: 521 - protein, 52 aa, Eutrema salsugineum
QQICEARSINFRGMCMKWRNCQRVCMSEGFPDGRCAGFIRRCICRKPCPILS

SEQ ID NO: 522 - protein, 100 aa, Eutrema salsugineum
RMCESSSQLFNGPCLSTTNCANICHNEGFTDGDCQGFRRRCICTRPCTPRGMERKTLGLFFMLFLLLSADKYYFMDADVALKTAEARRCESKSHKFKGPC

SEQ ID NO: 523 - protein, 48 aa, Trifolium pratense
RTCESQSHKFKGPCASDHNCASVCQTERFPGGHCRGFRRRCFCTTHCS

SEQ ID NO: 524 - protein, 55 aa, Trifolium pratense
EMVVETEGRHCESKSHRFKGMCLSDHNCASVCHVEGFPGGNCRGFRRRCFCKKRC

SEQ ID NO: 525 - protein, 48 aa, Trifolium pratense
RKCESKRRWIYGNCLSDEKCASDCLVKGFLFGKCRGYDNRCYCKKPRC

SEQ ID NO: 526 - protein, 47 aa, Tripterygium wilfordii
RVCASKSHYYKGPCLHNHNCAMVCRNEGFSGGKCHGLGRRCFCTKLC

SEQ ID NO: 527 - protein, 47 aa, Tripterygium wilfordii
RVCLSPSHKFHGTCVRGHNCAEICRTEGFSGGDCHGFRRRCMCSRRC

SEQ ID NO: 528 - protein, 57 aa, Phtheirospermum japonicum
AGEEAVGEEKKACKYKSEKFWGICITDSKCIEVCRREGFQTGDCKGLRRRCYCVNNC

SEQ ID NO: 529 - protein, 82 aa, Vigna mungo
VASGGKDKTKKPGRYIATELENIETNYFSDYTGFEIQIDSTDVDDAIDTLSEVASKKLVQILLRFVALFLPVAPIPPLPIPS

SEQ ID NO: 530 - protein, 48 aa, Zea mays
RTCQSQSHRFRGPCLRRSNCANVCRTEGFPGGRCRGFRRRCFCTTHCH

SEQ ID NO: 531 - protein, 55 aa, Zea mays
RVVVEETLCLSQSHAFKGVCLSNTNCDNVCKTEKFTGGECKMDGVMRKCYCKKVC

SEQ ID NO: 532 - protein, 49 aa, Zea mays
RHCLSQSHRFKGLCMSSNNCANVCQTENFPGGECKAEGATRKCFCKKIC

SEQ ID NO: 533 - protein, 55 aa, Zea mays
GPVTMVSARKCESQSFRFKGPCSRDANCANVCLTEGFTGGVCKGLRHRCFCTRDC

SEQ ID NO: 534 - protein, 49 aa, Pinus sylvestris
RMCKTPSGKFKGYCVSSTNCKNVCRTEGFPTGSCDFHVASRKCYCYKPC

SEQ ID NO: 535 - protein, 48 aa, synthetic construct
  VARIANT 43: note = W, F, or Y
  VARIANT 45: note = W, F, or Y
RMCKTPSGKFKGYCVSSTNCKNVCRTEGFPTGSCDFHVASRKXYXKPC

SEQ ID NO: 536 - protein, 49 aa, synthetic construct
RMCKTPSGKFKGYCVSSTNCKNVCRTEGFPTGSCDFHVASRKWFWYKPC

SEQ ID NO: 537 - protein, 17 aa, synthetic construct
  VARIANT 12: note = W, F, or Y
  VARIANT 14: note = W, F, or Y
GSCDFHVASRKXYXKPC

SEQ ID NO: 538 - protein, 50 aa, Pinus sylvestris
RTCLSKSHRFHGYCVRSSSCKEICQTERFLSGECRHTHLGNRRCFCEKPC

SEQ ID NO: 539 - protein, 50 aa, synthetic construct
  VARIANT 44: note = W, F, or Y
  VARIANT 46: note = W, F, or Y
RTCLSKSHRFHGYCVRSSSCKEICQTERFLSGECRHTHLGNRRXFXEKPC

SEQ ID NO: 540 - protein, 50 aa, synthetic construct
RTCLSKSHRFHGYCVRSSSCKEICQTERFLSGECRHTHLGNRRWFWEKPC

SEQ ID NO: 541 - protein, 11 aa, synthetic construct
  VARIANT 5: note = W, F, or Y
  VARIANT 7: note = W, F, or Y
GNRRXFXEKPC

SEQ ID NO: 542 - protein, 47 aa, Eucalyptus grandis
RMCESQSHKFHGLCMSDRTCVVVCKEEDFDDGKCRGFRHRCFCRKPC

SEQ ID NO: 543 - protein, 47 aa, synthetic construct
  VARIANT 41: note = W, F, or Y
  VARIANT 43: note = W, F, or Y
RMCESQSHKFHGLCMSDRTCVVVCKEEDFDDGKCRGFRHRXFXRKPC

SEQ ID NO: 544 - protein, 47 aa, synthetic construct
RMCESQSHKFHGLCMSDRTCVVVCKEEDFDDGKCRGFRHRWFWRKPC

SEQ ID NO: 545 - protein, 16 aa, synthetic construct
  VARIANT 10: note = W, F, or Y
  VARIANT 12: note = W, F, or Y
GKCRGFRHRXFXRKPC

SEQ ID NO: 546 - protein, 47 aa, Striga asiatica
KICEYKSERFWGVCLSDIKCADVCKDESFESGDCQGLRRRCMCYNHC

SEQ ID NO: 547 - protein, 47 aa, synthetic construct
  VARIANT 41: note = W, F, or Y
  VARIANT 43: note = W, F, or Y
KICEYKSERFWGVCLSDIKCADVCKDESFESGDCQGLRRRXMXYNHC

SEQ ID NO: 548 - protein, 47 aa, synthetic construct
  VARIANT 41..43: note = W, F, or Y
KICEYKSERFWGVCLSDIKCADVCKDESFESGDCQGLRRRXXXYNHC

SEQ ID NO: 549 - protein, 47 aa, synthetic construct
KICEYKSERFWGVCLSDIKCADVCKDESFESGDCQGLRRRWFWYNHC

SEQ ID NO: 550 - protein, 16 aa, synthetic construct
  VARIANT 10..12: note = W, F, or Y
GDCQGLRRRXXXYNHC

SEQ ID NO: 551 - protein, 49 aa, Panax ginseng
KLCEKASLTWSGKCGNTQNCDKQCQNWESAKHGACHKRPTWKCFCYSDC

SEQ ID NO: 552 - protein, 49 aa, synthetic construct
  VARIANT 43: note = W, F, or Y
  VARIANT 45: note = W, F, or Y
KLCEKASLTWSGKCGNTQNCDKQCQNWESAKHGACHKRPTWKXFXYSDC

SEQ ID NO: 553 - protein, 49 aa, synthetic construct
KLCEKASLTWSGKCGNTQNCDKQCQNWESAKHGACHKRPTWKWFWYSDC

SEQ ID NO: 554 - protein, 17 aa, synthetic construct
  VARIANT 11: note = W, F, or Y
  VARIANT 13: note = W, F, or Y
GACHKRPTWKXFXYSDC

SEQ ID NO: 555 - protein, 45 aa, Erythranthe guttata
SLCETPSRYFNGICFSRRNCGAICEKEGFSSGRCKAFKCVCGKDC

SEQ ID NO: 556 - protein, 45 aa, synthetic construct
  VARIANT 39: note = W, F, or Y
  VARIANT 41: note = W, F, or Y
SLCETPSRYFNGICFSRRNCGAICEKEGFSSGRCKAFKXVXGKDC

SEQ ID NO: 557 - protein, 45 aa, synthetic construct
  VARIANT 39..41: note = W, F, or Y
SLCETPSRYFNGICFSRRNCGAICEKEGFSSGRCKAFKXXXGKDC

SEQ ID NO: 558 - protein, 45 aa, synthetic construct
SLCETPSRYFNGICFSRRNCGAICEKEGFSSGRCKAFKWFWGKDC

SEQ ID NO: 559 - protein, 14 aa, synthetic construct
  VARIANT 8: note = W, F, or Y
  VARIANT 10: note = W, F, or Y
GRCKAFKXVXGKDC

SEQ ID NO: 560 - protein, 50 aa, Raphanus sativus
KLCQRPSGTWSGVCGNNNACKNQCIRLEKARHGSCNYVFPAHKCICYFPC

SEQ ID NO: 561 - protein, 50 aa, synthetic construct
  VARIANT 44: note = W, F, or Y
  VARIANT 46: note = W, F, or Y
KLCQRPSGTWSGVCGNNNACKNQCIRLEKARHGSCNYVFPAHKXIXYFPC

SEQ ID NO: 562 - protein, 50 aa, synthetic construct
  VARIANT 44..46: note = W, F, or Y
KLCQRPSGTWSGVCGNNNACKNQCIRLEKARHGSCNYVFPAHKXXXYFPC

SEQ ID NO: 563 - protein, 50 aa, synthetic construct
KLCQRPSGTWSGVCGNNNACKNQCIRLEKARHGSCNYVFPAHKWFWYFPC

SEQ ID NO: 564 - protein, 18 aa, synthetic construct
  VARIANT 12..14: note = W, F, or Y
GSCNYVFPAHKXXXYFPC

SEQ ID NO: 565 - protein, 52 aa, Fraxinus excelsior
KHCAKLSKGWHGFCAPHKCSRYCIHHEGAYDGACLKHHHSKHIGCYCYYRHC

SEQ ID NO: 566 - protein, 52 aa, synthetic construct
  VARIANT 45: note = W, F, or Y
  VARIANT 47: note = W, F, or Y
KHCAKLSKGWHGFCAPHKCSRYCIHHEGAYDGACLKHHHSKHIGXYXYYRHC

SEQ ID NO: 567 - protein, 52 aa, synthetic construct
KHCAKLSKGWHGFCAPHKCSRYCIHHEGAYDGACLKHHHSKHIGWFWYYRHC

SEQ ID NO: 568 - protein, 21 aa, synthetic construct
  VARIANT 14: note = W, F, or Y
  VARIANT 16: note = W, F, or Y
GACLKHHHSKHIGXYXYYRHC

SEQ ID NO: 569 - protein, 48 aa, Crambe maritima
KLCRKPSGTWSGLCANSDSCRKQCINLEKAKDGSCKYKFPAHKCFCYY

SEQ ID NO: 570 - protein, 48 aa, synthetic construct
  VARIANT 44: note = W, F, or Y
  VARIANT 46: note = W, F, or Y
KLCRKPSGTWSGLCANSDSCRKQCINLEKAKDGSCKYKFPAHKXFXYY

SEQ ID NO: 571 - protein, 48 aa, synthetic construct
KLCRKPSGTWSGLCANSDSCRKQCINLEKAKDGSCKYKFPAHKWFWYY

SEQ ID NO: 572 - protein, 16 aa, synthetic construct
  VARIANT 12: note = W, F, or Y
  VARIANT 14: note = W, F, or Y
GSCKYKFPAHKXFXYY

SEQ ID NO: 573 - protein, 48 aa, Chlorophytum laxum
ARVCRRRSAGFRGLCVSNHNCAQVCMQEGWGGGNCDGFRRQCKCMRQC

SEQ ID NO: 574 - protein, 48 aa, synthetic construct
  VARIANT 42: note = W, F, or Y
  VARIANT 44: note = W, F, or Y
ARVCRRRSAGFRGLCVSNHNCAQVCMQEGWGGGNCDGFRRQXKXMRQC

SEQ ID NO: 575 - protein, 48 aa, synthetic construct
  VARIANT 42..44: note = W, F, or Y
ARVCRRRSAGFRGLCVSNHNCAQVCMQEGWGGGNCDGFRRQXXXMRQC

SEQ ID NO: 576 - protein, 48 aa, synthetic construct
ARVCRRRSAGFRGLCVSNHNCAQVCMQEGWGGGNCDGFRRQWFWMRQC

SEQ ID NO: 577 - protein, 16 aa, synthetic construct
  VARIANT 10..12: note = W, F, or Y
GNCDGFRRQXXXMRQC

SEQ ID NO: 578 - protein, 65 aa, Setaria viridis
AYMCTTQNRFFHGRCVNNLNCASSCVHGRLGTGGHCATRKRSAADPGVDIDWVWNRRICVCVFQC

SEQ ID NO: 579 - protein, 65 aa, synthetic construct
  VARIANT 59: note = W, F, or Y
  VARIANT 61: note = W, F, or Y
AYMCTTQNRFFHGRCVNNLNCASSCVHGRLGTGGHCATRKRSAADPGVDIDWVWNRRIXVXVFQC

SEQ ID NO: 580 - protein, 65 aa, synthetic construct
  VARIANT 59..61: note = W, F, or Y
AYMCTTQNRFFHGRCVNNLNCASSCVHGRLGTGGHCATRKRSAADPGVDIDWVWNRRIXXXVFQC

SEQ ID NO: 581 - protein, 65 aa, synthetic construct
AYMCTTQNRFFHGRCVNNLNCASSCVHGRLGTGGHCATRKRSAADPGVDIDWVWNRRIWFWVFQC

SEQ ID NO: 582 - protein, 32 aa, synthetic construct
  VARIANT 26..28: note = W, F, or Y
GHCATRKRSAADPGVDIDWVWNRRIXXXVFQC

SEQ ID NO: 583 - protein, 48 aa, Trifolium pratense; identical to SEQ ID NO: 525

SEQ ID NO: 584 - protein, 48 aa, synthetic construct
  VARIANT 41: note = W, F, or Y
  VARIANT 43: note = W, F, or Y
RKCESKRRWIYGNCLSDEKCASDCLVKGFLFGKCRGYDNRXYXKKPRC

SEQ ID NO: 585 - protein, 48 aa, synthetic construct
  VARIANT 41..43: note = W, F, or Y
RKCESKRRWIYGNCLSDEKCASDCLVKGFLFGKCRGYDNRXXXKKPRC

SEQ ID NO: 586 - protein, 48 aa, synthetic construct
RKCESKRRWIYGNCLSDEKCASDCLVKGFLFGKCRGYDNRWFWKKPRC

SEQ ID NO: 587 - protein, 17 aa, synthetic construct
  VARIANT 10..12: note = W, F, or Y
GKCRGYDNRXXXKKPRC

SEQ ID NO: 588 - protein, 48 aa, Fragaria vesca; identical to SEQ ID NO: 470

SEQ ID NO: 589 - protein, 48 aa, synthetic construct
  VARIANT 41: note = W, F, or Y
  VARIANT 43: note = W, F, or Y
KICEVPSKTFKGLCVKDRNCVVRCHSEGYGNGKCSHVRRQXMXLKICD

SEQ ID NO: 590 - protein, 48 aa, synthetic construct
  VARIANT 41..43: note = W, F, or Y
KICEVPSKTFKGLCVKDRNCVVRCHSEGYGNGKCSHVRRQXXXLKICD

SEQ ID NO: 591 - protein, 48 aa, synthetic construct
KICEVPSKTFKGLCVKDRNCVVRCHSEGYGNGKCSHVRRQWFWLKICD

SEQ ID NO: 592 - protein, 17 aa, synthetic construct
  VARIANT 10..12: note = W, F, or Y
GKCSHVRRQXXXLKICD

SEQ ID NO: 593 - protein, 48 aa, Solanum lycopersicum
KLCQYHSETFHGVCVTGNNCNRHCQRENFEGGRCHGIRHYRCVCYRTC

SEQ ID NO: 594 - protein, 48 aa, synthetic construct
  VARIANT 42: note = W, F, or Y
  VARIANT 44: note = W, F, or Y
KLCQYHSETFHGVCVTGNNCNRHCQRENFEGGRCHGIRHYRXVXYRTC

SEQ ID NO: 595 - protein, 48 aa, synthetic construct
  VARIANT 42..44: note = W, F, or Y
KLCQYHSETFHGVCVTGNNCNRHCQRENFEGGRCHGIRHYRXXXYRTC

SEQ ID NO: 596 - protein, 48 aa, synthetic construct
KLCQYHSETFHGVCVTGNNCNRHCQRENFEGGRCHGIRHYRWFWYRTC

SEQ ID NO: 597 - protein, 17 aa, synthetic construct
  VARIANT 11..13: note = W, F, or Y
GRCHGIRHYRXXXYRTC

SEQ ID NO: 598 - protein, 47 aa, Mimulus guttatus
RHCESPSHRFKGICVRKSNCAAICQTEGFSSGKCRGFRSRCVCIKHC

SEQ ID NO: 599 - protein, 47 aa, synthetic construct
  VARIANT 41: note = W, F, or Y
  VARIANT 43: note = W, F, or Y
RHCESPSHRFKGICVRKSNCAAICQTEGFSSGKCRGFRSRXVXIKHC

SEQ ID NO: 600 - protein, 47 aa, synthetic construct
  VARIANT 41..43: note = W, F, or Y
RHCESPSHRFKGICVRKSNCAAICQTEGFSSGKCRGFRSRXXXIKHC

SEQ ID NO: 601 - protein, 47 aa, synthetic construct
RHCESPSHRFKGICVRKSNCAAICQTEGFSSGKCRGFRSRWFWIKHC

SEQ ID NO: 602 - protein, 16 aa, synthetic construct
  VARIANT 10..12: note = W, F, or Y
GKCRGFRSRXXXIKHC

SEQ ID NO: 603 - protein, 51 aa, Thlaspi arvense
RMCESKSHEFKGFCTKWRICRDVCITEGFTDGGCHGVLRHCVCKKPCLLSN

SEQ ID NO: 604 - protein, 51 aa, synthetic construct
  VARIANT 41: note = W, F, or Y
  VARIANT 43: note = W, F, or Y
RMCESKSHEFKGFCTKWRICRDVCITEGFTDGGCHGVLRHXVXKKPCLLSN

SEQ ID NO: 605 - protein, 51 aa, synthetic construct
  VARIANT 41..43: note = W, F, or Y
RMCESKSHEFKGFCTKWRICRDVCITEGFTDGGCHGVLRHXXXKKPCLLSN

SEQ ID NO: 606 - protein, 51 aa, synthetic construct
RMCESKSHEFKGFCTKWRICRDVCITEGFTDGGCHGVLRHWFWKKPCLLSN

SEQ ID NO: 607 - protein, 16 aa, synthetic construct
  VARIANT 6..8: note = W, F, or Y
GVLRHXXXKKPCLLSN

SEQ ID NO: 608 - protein, 65 aa, Rubus chingii
GACPLVSKRSKTWSGFCGSSNNCNKQCRSWESAQHGACHNTYKTVLGVKVISGRACFCYFCKKHT

SEQ ID NO: 609 - protein, 65 aa, synthetic construct
  VARIANT 56: note = W, F, or Y
  VARIANT 58: note = W, F, or Y
GACPLVSKRSKTWSGFCGSSNNCNKQCRSWESAQHGACHNTYKTVLGVKVISGRAXFXYFCKKHT

SEQ ID NO: 610 - protein, 65 aa, synthetic construct
GACPLVSKRSKTWSGFCGSSNNCNKQCRSWESAQHGACHNTYKTVLGVKVISGRAWFWYFCKKHT

SEQ ID NO: 611 - protein, 30 aa, synthetic construct
  VARIANT 21: note = W, F, or Y
  VARIANT 23: note = W, F, or Y
GACHNTYKTVLGVKVISGRAXFXYFCKKHT

SEQ ID NO: 612 - protein, 64 aa, Prunus dulcis
VTCPLESKRSKTWSGVCIKSKNCDKQCKTWERAKHGACHATYRNVLGVKVFKGFACFCYFCKKH

SEQ ID NO: 613 - protein, 64 aa, synthetic construct
  VARIANT 56: note = W, F, or Y
  VARIANT 58: note = W, F, or Y
VTCPLESKRSKTWSGVCIKSKNCDKQCKTWERAKHGACHATYRNVLGVKVFKGFAXFXYFCKKH

SEQ ID NO: 614 - protein, 64 aa, synthetic construct
VTCPLESKRSKTWSGVCIKSKNCDKQCKTWERAKHGACHATYRNVLGVKVFKGFAWFWYFCKKH

SEQ ID NO: 615 - protein, 29 aa, synthetic construct
  VARIANT 21: note = W, F, or Y
  VARIANT 23: note = W, F, or Y
GACHATYRNVLGVKVFKGFAXFXYFCKKH

SEQ ID NO: 616 - protein, 50 aa, Theobroma cacao
KLCQKRSKTWTGPCIKTKNCDHQCRKWEKAQHGACHWQWPGFACFCYVNC

SEQ ID NO: 617 - protein, 50 aa, synthetic construct
  VARIANT 44: note = W, F, or Y
  VARIANT 46: note = W, F, or Y
KLCQKRSKTWTGPCIKTKNCDHQCRKWEKAQHGACHWQWPGFAXFXYVNC

SEQ ID NO: 618 - protein, 50 aa, synthetic construct
KLCQKRSKTWTGPCIKTKNCDHQCRKWEKAQHGACHWQWPGFAWFWYVNC

SEQ ID NO: 619 - protein, 18 aa, synthetic construct
  VARIANT 12: note = W, F, or Y
  VARIANT 14: note = W, F, or Y
GACHWQWPGFAXFXYVNC

SEQ ID NO: 620 - protein, 49 aa, Prunus armeniaca
QRGCKQASKSMTTCTAHPECDKKCKGEKAKMGLCRADGPKGKKCWCYMC

SEQ ID NO: 621 - protein, 49 aa, synthetic construct
  VARIANT 44: note = W, F, or Y
  VARIANT 46: note = W, F, or Y
QRGCKQASKSMTTCTAHPECDKKCKGEKAKMGLCRADGPKGKKXWXYMC

SEQ ID NO: 622 - protein, 49 aa, synthetic construct
QRGCKQASKSMTTCTAHPECDKKCKGEKAKMGLCRADGPKGKKWFWYMC

SEQ ID NO: 623 - protein, 18 aa, synthetic construct
  VARIANT 13: note = W, F, or Y
  VARIANT 15: note = W, F, or Y
GLCRADGPKGKKXWXYMC

SEQ ID NO: 624 - protein, 52 aa, Nicotiana sylvestris
KLCRRRSKTFSSSCFISEHCDKECKEKEGAKRGMCIKKSIFGRFCYCFHKCK

SEQ ID NO: 625 - protein, 52 aa, synthetic construct
  VARIANT 45: note = W, F, or Y
  VARIANT 47: note = W, F, or Y
KLCRRRSKTFSSSCFISEHCDKECKEKEGAKRGMCIKKSIFGRFXYXFHKCK

SEQ ID NO: 626 - protein, 52 aa, synthetic construct
KLCRRRSKTFSSSCFISEHCDKECKEKEGAKRGMCIKKSIFGRFWFWFHKCK

SEQ ID NO: 627 - protein, 20 aa, synthetic construct
  VARIANT 13: note = W, F, or Y
  VARIANT 15: note = W, F, or Y
GMCIKKSIFGRFXYXFHKCK

SEQ ID NO: 628 - protein, 49 aa, Cajanus cajan; identical to SEQ ID NO: 106

SEQ ID NO: 629 - protein, 49 aa, synthetic construct
  VARIANT 41: note = W, F, or Y
  VARIANT 43: note = W, F, or Y
AVCSKPSTYFFGPCVRRSTCRRACSHENYPDGKCSRFLGKXIXYKPCTT

SEQ ID NO: 630 - protein, 49 aa, synthetic construct
  VARIANT 41..43: note = W, F, or Y
AVCSKPSTYFFGPCVRRSTCRRACSHENYPDGKCSRFLGKXXXYKPCTT

SEQ ID NO: 631 - protein, 49 aa, synthetic construct
AVCSKPSTYFFGPCVRRSTCRRACSHENYPDGKCSRFLGKWFWYKPCTT

SEQ ID NO: 632 - protein, 18 aa, synthetic construct
  VARIANT 10..12: note = W, F, or Y
GKCSRFLGKXXXYKPCTT

SEQ ID NO: 633 - protein, 46 aa, Eucalyptus grandis
ATCRKPSMYFSGACFSRTNCQKACNREDWPNGKCLVGFKCFCQRPC

SEQ ID NO: 634 - protein, 46 aa, synthetic construct
  VARIANT 40: note = W, F, or Y
  VARIANT 42: note = W, F, or Y
ATCRKPSMYFSGACFSRTNCQKACNREDWPNGKCLVGFKXFXQRPC

SEQ ID NO: 635 - protein, 46 aa, synthetic construct
ATCRKPSMYFSGACFSRTNCQKACNREDWPNGKCLVGFKWFWQRPC

SEQ ID NO: 636 - protein, 15 aa, synthetic construct
  VARIANT 9: note = W, F, or Y
  VARIANT 11: note = W, F, or Y
GKCLVGFKXFXQRPC

SEQ ID NO: 637 - protein, 46 aa, Eucalyptus grandis; identical to SEQ ID NO: 21

SEQ ID NO: 638 - protein, 46 aa, synthetic construct
  VARIANT 40..42: note = W, F, or Y
AICKKPSKFFKGACGRDADCEKACDQENWPGGVCVPFLRXXXQRSC

SEQ ID NO: 639 - protein, 46 aa, synthetic construct
AICKKPSKFFKGACGRDADCEKACDQENWPGGVCVPFLRWFWQRSC

SEQ ID NO: 640 - protein, 15 aa, synthetic construct
  VARIANT 9..11: note = W, F, or Y
GVCVPFLRXXXQRSC

SEQ ID NO: 641 - protein, 49 aa, Eucalyptus grandis; identical to SEQ ID NO: 109

SEQ ID NO: 642 - protein, 49 aa, synthetic construct
  VARIANT 43..45: note = W, F, or Y
ATCAKPSKYYKGGCFATISVSACKKTCSRENWPDGACVYPFRXXXRRPC

SEQ ID NO: 643 - protein, 49 aa, synthetic construct
ATCAKPSKYYKGGCFATISVSACKKTCSRENWPDGACVYPFRWFWRRPC

SEQ ID NO: 644 - protein, 15 aa, synthetic construct
  VARIANT 9..11: note = W, F, or Y
GACVYPFRXXXRRPC

SEQ ID NO: 645 - protein, 49 aa, Eucalyptus grandis; identical to SEQ ID NO: 111

SEQ ID NO: 646 - protein, 49 aa, synthetic construct
  VARIANT 43..45: note = W, F, or Y
ANCAKPSKFYRGPCFNSISVKPCTQRCSQENWPIGVCTKDLRXXXQRPC

SEQ ID NO: 647 - protein, 49 aa, synthetic construct
ANCAKPSKFYRGPCFNSISVKPCTQRCSQENWPIGVCTKDLRWFWQRPC

SEQ ID NO: 648 - protein, 15 aa, synthetic construct
  VARIANT 9..11: note = W, F, or Y
GVCTKDLRXXXQRPC

SEQ ID NO: 649 - protein, 47 aa, Fragaria X ananassa; identical to SEQ ID NO: 246

SEQ ID NO: 650 - protein, 47 aa, synthetic construct
  VARIANT 41..43: note = W, F, or Y
RVCEVPSKTFTGLCFRDTHCIVRCHSEGYGYGKCSHVLRRXXXLKAC

SEQ ID NO: 651 - protein, 47 aa, synthetic construct
RVCEVPSKTFTGLCFRDTHCIVRCHSEGYGYGKCSHVLRRWFWLKAC

SEQ ID NO: 652 - protein, 16 aa, synthetic construct
  VARIANT 10..12: note = W, F, or Y
GKCSHVLRRXXXLKAC

SEQ ID NO: 653 - protein, 48 aa, Fragaria X ananassa
KTCESPSAKFEGPCIMGSNCALTCQKEGHDGGFCRDFWLPKCFCTKPC

SEQ ID NO: 654 - protein, 48 aa, synthetic construct
  VARIANT 42: note = W, F, or Y
  VARIANT 44: note = W, F, or Y
KTCESPSAKFEGPCIMGSNCALTCQKEGHDGGFCRDFWLPKXFXTKPC

SEQ ID NO: 655 - protein, 48 aa, synthetic construct
KTCESPSAKFEGPCIMGSNCALTCQKEGHDGGFCRDFWLPKWFWTKPC

SEQ ID NO: 656 - protein, 17 aa, synthetic construct
  VARIANT 11: note = W, F, or Y
  VARIANT 13: note = W, F, or Y
GFCRDFWLPKXFXTKPC

SEQ ID NO: 657 - protein, 47 aa, Fragaria X ananassa
ITCKTQSIKYEGMCSREKPCASLCQTKGYSRGRCHGVYKICVCATQC

SEQ ID NO: 658 - protein, 47 aa, synthetic construct
  VARIANT 41: note = W, F, or Y
  VARIANT 43: note = W, F, or Y
ITCKTQSIKYEGMCSREKPCASLCQTKGYSRGRCHGVYKIXVXATQC

SEQ ID NO: 659 - protein, 47 aa, synthetic construct
  VARIANT 41..43: note = W, F, or Y
ITCKTQSIKYEGMCSREKPCASLCQTKGYSRGRCHGVYKIXXXATQC

SEQ ID NO: 660 - protein, 47 aa, synthetic construct
ITCKTQSIKYEGMCSREKPCASLCQTKGYSRGRCHGVYKIWFWATQC

SEQ ID NO: 661 - protein, 16 aa, synthetic construct
  VARIANT 10..12: note = W, F, or Y
GRCHGVYKIXXXATQC

SEQ ID NO: 662 - protein, 48 aa, Fragaria vesca; identical to SEQ ID NO: 259

SEQ ID NO: 663 - protein, 48 aa, synthetic construct
  VARIANT 41: note = W, F, or Y
  VARIANT 43: note = W, F, or Y
SICEIPSKKFKGLCIRDRKCIVICHSEGYGYGKCSHIGRRXMXLKTCD

SEQ ID NO: 664 - protein, 48 aa, synthetic construct
SICEIPSKKFKGLCIRDRKCIVICHSEGYGYGKCSHIGRRWFWLKTCD

SEQ ID NO: 665 - protein, 17 aa, synthetic construct
  VARIANT 10..12: note = W, F, or Y
GKCSHIGRRXXXLKTCD

SEQ ID NO: 666 - protein, 47 aa, Glycine max; identical to SEQ ID NO: 32

SEQ ID NO: 667 - protein, 47 aa, synthetic construct
  VARIANT 41..43: note = W, F, or Y
KDCLTRRHGFQGRCLFDRQCAHVCRSDGFIGGQCRGPLRKXXXSRPC

SEQ ID NO: 668 - protein, 47 aa, synthetic construct
KDCLTRRHGFQGRCLFDRQCAHVCRSDGFIGGQCRGPLRKWFWSRPC

SEQ ID NO: 669 - protein, 16 aa, synthetic construct
  VARIANT 10..12: note = W, F, or Y
GQCRGPLRKXXXSRPC

SEQ ID NO: 670 - protein, 48 aa, Gossypium hirsutum
RICQSRSTEYRGMCLFDVNCDNICKTEPGFTGGHCHSFFRSCYCTKPC

SEQ ID NO: 671 - protein, 48 aa, synthetic construct
  VARIANT 42..44: note = W, F, or Y
RICQSRSTEYRGMCLFDVNCDNICKTEPGFTGGHCHSFFRSXXXTKPC

SEQ ID NO: 672 - protein, 48 aa, synthetic construct
RICQSRSTEYRGMCLFDVNCDNICKTEPGFTGGHCHSFFRSWFWTKPC

SEQ ID NO: 673 - protein, 16 aa, synthetic construct
  VARIANT 10..12: note = W, F, or Y
GHCHSFFRSXXXTKPC

SEQ ID NO: 674 - protein, 55 aa, Gossypium hirsutum
GPTIVGGRKCKSPSKKFNGMCMNHVNCATVCQTESHEDGKCEGFYRRCICIKPCF

SEQ ID NO: 675 - protein, 55 aa, synthetic construct
  VARIANT 48: note = W, F, or Y
  VARIANT 50: note = W, F, or Y
GPTIVGGRKCKSPSKKFNGMCMNHVNCATVCQTESHEDGKCEGFYRRXIXIKPCF

SEQ ID NO: 676 - protein, 55 aa, synthetic construct
  VARIANT 48..50: note = W, F, or Y
GPTIVGGRKCKSPSKKFNGMCMNHVNCATVCQTESHEDGKCEGFYRRXXXIKPCF

SEQ ID NO: 677 - protein, 55 aa, synthetic construct
GPTIVGGRKCKSPSKKFNGMCMNHVNCATVCQTESHEDGKCEGFYRRWFWIKPCF

SEQ ID NO: 678 - protein, 17 aa, synthetic construct
  VARIANT 10..12: note = W, F, or Y
GKCEGFYRRXXXIKPCF

SEQ ID NO: 679 - protein, 48 aa, Lupinus albus
RTCKSKSQRYKGSCYSDNNCAIICEGEGFTGGKCHGFFCKCWCKRPCL

SEQ ID NO: 680 - protein, 48 aa, synthetic construct
  VARIANT 41: note = W, F, or Y
  VARIANT 43: note = W, F, or Y
RTCKSKSQRYKGSCYSDNNCAIICEGEGFTGGKCHGFFCKXWXKRPCL

SEQ ID NO: 681 - protein, 48 aa, synthetic construct
RTCKSKSQRYKGSCYSDNNCAIICEGEGFTGGKCHGFFCKWFWKRPCL

SEQ ID NO: 682 - protein, 17 aa, synthetic construct
  VARIANT 10: note = W, F, or Y
  VARIANT 12: note = W, F, or Y
GKCHGFFCKXWXKRPCL

SEQ ID NO: 683 - protein, 48 aa, Lupinus albus; identical to SEQ ID NO: 323

SEQ ID NO: 684 - protein, 48 aa, synthetic construct
  VARIANT 42: note = W, F, or Y
  VARIANT 44: note = W, F, or Y
NNCKYESNKFHGVCLSDTNCESVCQTEGFPFGKCDGVLPRTXVXKKPC

SEQ ID NO: 685 - protein, 48 aa, synthetic construct
  VARIANT 42..44: note = W, F, or Y
NNCKYESNKFHGVCLSDTNCESVCQTEGFPFGKCDGVLPRTXXXKKPC

SEQ ID NO: 686 - protein, 48 aa, synthetic construct
NNCKYESNKFHGVCLSDTNCESVCQTEGFPFGKCDGVLPRTWFWKKPC

SEQ ID NO: 687 - protein, 17 aa, synthetic construct
  VARIANT 11..13: note = W, F, or Y
GKCDGVLPRTXXXKKPC

SEQ ID NO: 688 - protein, 42 aa, Manihot esculenta
HTCHKKIDIKTCDFQKCNKECAKETLGVGDCRNALCFCTYYC

SEQ ID NO: 689 - protein, 42 aa, synthetic construct
  VARIANT 36: note = W, F, or Y
  VARIANT 38: note = W, F, or Y
HTCHKKIDIKTCDFQKCNKECAKETLGVGDCRNALXFXTYYC

SEQ ID NO: 690 - protein, 42 aa, synthetic construct
HTCHKKIDIKTCDFQKCNKECAKETLGVGDCRNALWFWTYYC

SEQ ID NO: 691 - protein, 14 aa, synthetic construct
  VARIANT 8: note = W, F, or Y
  VARIANT 10: note = W, F, or Y
GDCRNALXFXTYYC

SEQ ID NO: 692 - protein, 48 aa, Manihot esculenta
RICVKPEPLAHCTETICTQFCIRKYGPPPGTVGKCNLPPGFCTCYHNC

SEQ ID NO: 693 - protein, 48 aa, synthetic construct
  VARIANT 42: note = W, F, or Y
  VARIANT 44: note = W, F, or Y
RICVKPEPLAHCTETICTQFCIRKYGPPPGTVGKCNLPPGFXTXYHNC

SEQ ID NO: 694 - protein, 48 aa, synthetic construct
  VARIANT 42..44: note = W, F, or Y
RICVKPEPLAHCTETICTQFCIRKYGPPPGTVGKCNLPPGFXXXYHNC

SEQ ID NO: 695 - protein, 48 aa, synthetic construct
RICVKPEPLAHCTETICTQFCIRKYGPPPGTVGKCNLPPGFWFWYHNC

SEQ ID NO: 696 - protein, 16 aa, synthetic construct
  VARIANT 10..12: note = W, F, or Y
GKCNLPPGFXXXYHNC

SEQ ID NO: 697 - protein, 47 aa, Malus domestica
KICEFPSTLFKGLCFSSNNCKHTCRKEQFTRGKCSLLTRTCMCTKKC

SEQ ID NO: 698 - protein, 47 aa, synthetic construct
  VARIANT 41: note = W, F, or Y
  VARIANT 43: note = W, F, or Y
KICEFPSTLFKGLCFSSNNCKHTCRKEQFTRGKCSLLTRTXMXTKKC

SEQ ID NO: 699 - protein, 47 aa, synthetic construct
  VARIANT 41..43: note = W, F, or Y
KICEFPSTLFKGLCFSSNNCKHTCRKEQFTRGKCSLLTRTXXXTKKC

SEQ ID NO: 700 - protein, 47 aa, synthetic construct
KICEFPSTLFKGLCFSSNNCKHTCRKEQFTRGKCSLLTRTWFWTKKC

SEQ ID NO: 701 - protein, 16 aa, synthetic construct
  VARIANT 10..12: note = W, F, or Y
GKCSLLTRTXXXTKKC

SEQ ID NO: 702 - protein, 49 aa, Setaria italica
RHCLSQSHTFKGLCFSSENCASVCKSEKFPGGQCQMHGASRKCFCKVVC

SEQ ID NO: 703 - protein, 49 aa, synthetic construct
  VARIANT 43: note = W, F, or Y
  VARIANT 45: note = W, F, or Y
RHCLSQSHTFKGLCFSSENCASVCKSEKFPGGQCQMHGASRKXFXKVVC

SEQ ID NO: 704 - protein, 49 aa, synthetic construct
RHCLSQSHTFKGLCFSSENCASVCKSEKFPGGQCQMHGASRKWFWKVVC

SEQ ID NO: 705 - protein, 18 aa, synthetic construct
  VARIANT 12: note = W, F, or Y
  VARIANT 14: note = W, F, or Y
GQCQMHGASRKXFXKVVC

SEQ ID NO: 706 - protein, 47 aa, Setaria italica; identical to SEQ ID NO: 43

SEQ ID NO: 707 - protein, 47 aa, synthetic construct
  VARIANT 41: note = W, F, or Y
  VARIANT 43: note = W, F, or Y
DRCETHSRTYKGRCNNHNCWSICITEGNTGGFCKGTLLLKXMXTSEC

SEQ ID NO: 708 - protein, 47 aa, synthetic construct
  VARIANT 41..43: note = W, F, or Y
DRCETHSRTYKGRCNNHNCWSICITEGNTGGFCKGTLLLKXXXTSEC

SEQ ID NO: 709 - protein, 47 aa, synthetic construct
DRCETHSRTYKGRCNNHNCWSICITEGNTGGFCKGTLLLKWFWTSEC

SEQ ID NO: 710 - protein, 17 aa, synthetic construct
  VARIANT 11..13: note = W, F, or Y
GFCKGTLLLKXXXTSEC

SEQ ID NO: 711 - protein, 49 aa, Setaria italica
RHCLSQSHHFKGLCLSSSNCANVCRVERFPDGECQTTAGTRKCFCKRIC

SEQ ID NO: 712 - protein, 49 aa, synthetic construct
  VARIANT 43: note = W, F, or Y
  VARIANT 45: note = W, F, or Y
RHCLSQSHHFKGLCLSSSNCANVCRVERFPDGECQTTAGTRKXFXKRIC

SEQ ID NO: 713 - protein, 49 aa, synthetic construct
RHCLSQSHHFKGLCLSSSNCANVCRVERFPDGECQTTAGTRKWFWKRIC

SEQ ID NO: 714 - protein, 18 aa, synthetic construct
  VARIANT 12: note = W, F, or Y
  VARIANT 14: note = W, F, or Y
GECQTTAGTRKXFXKRIC

SEQ ID NO: 715 - protein, 50 aa, Setaria italica; identical to SEQ ID NO: 45

SEQ ID NO: 716 - protein, 50 aa, synthetic construct
  VARIANT 44: note = W, F, or Y
  VARIANT 46: note = W, F, or Y
RRCVSQSHKFVGSCMRKSNCQHVCQTEGFPWGECRFHGGLLRRXFXNKLC

SEQ ID NO: 717 - protein, 50 aa, synthetic construct
RRCVSQSHKFVGSCMRKSNCQHVCQTEGFPWGECRFHGGLLRRWFWNKLC

SEQ ID NO: 718 - protein, 19 aa, synthetic construct
  VARIANT 13: note = W, F, or Y
  VARIANT 15: note = W, F, or Y
GECRFHGGLLRRXFXNKLC

SEQ ID NO: 719 - protein, 47 aa, Striga asiatica
RKCESKSQQFKGLCFIKANCAAVCRTEGFHSGHCRGFRRRCYCTKHC

SEQ ID NO: 720 - protein, 47 aa, synthetic construct
  VARIANT 41: note = W, F, or Y
  VARIANT 43: note = W, F, or Y
RKCESKSQQFKGLCFIKANCAAVCRTEGFHSGHCRGFRRRXYXTKHC

SEQ ID NO: 721 - protein, 47 aa, synthetic construct
RKCESKSQQFKGLCFIKANCAAVCRTEGFHSGHCRGFRRRWFWTKHC

SEQ ID NO: 722 - protein, 16 aa, synthetic construct
  VARIANT 10: note = W, F, or Y
  VARIANT 12: note = W, F, or Y
GHCRGFRRRXYXTKHC

SEQ ID NO: 723 - protein, 48 aa, Rubus argutus
RICEVPSKTFIGLCFKDTNCIVRCRSEGYGYGKCSHFRRRCRCFKPCP

SEQ ID NO: 724 - protein, 48 aa, synthetic construct
  VARIANT 41: note = W, F, or Y
  VARIANT 43: note = W, F, or Y
RICEVPSKTFIGLCFKDTNCIVRCRSEGYGYGKCSHFRRRXRXFKPCP

SEQ ID NO: 725 - protein, 48 aa, synthetic construct
  VARIANT 41..43: note = W, F, or Y
RICEVPSKTFIGLCFKDTNCIVRCRSEGYGYGKCSHFRRRXXXFKPCP

SEQ ID NO: 726 - protein, 48 aa, synthetic construct
RICEVPSKTFIGLCFKDTNCIVRCRSEGYGYGKCSHFRRRWFWFKPCP

SEQ ID NO: 727 - protein, 17 aa, synthetic construct
  VARIANT 10..12: note = W, F, or Y
GKCSHFRRRXXXFKPCP

SEQ ID NO: 728 - protein, 57 aa, Prunus armeniaca
VICPLESKRSKTWSGVCAQSKNCDKQCKTWERAKHGACHATYGKVLGVKACMHAGNK

SEQ ID NO: 729 - protein, 22 aa, synthetic construct
GACHATYGKVLGVKACMHAGNK

SEQ ID NO: 730 - protein, 56 aa, Prunus armeniaca
KLCERRSKTWTGWCAKTSHCNKQCKDWERVEHGACHGEFLEKPVSAISNAHCKISF

SEQ ID NO: 731 - protein, 24 aa, synthetic construct
GACHGEFLEKPVSAISNAHCKISF

SEQ ID NO: 732 - protein, 63 aa, Potentilla micrantha
GACPLVQKRSKTWSGFCGSSNNCDKQCRTWEKAKHGACHATGDLKGTPRLKLRSCHARRSKDH

SEQ ID NO: 733 - protein, 28 aa, synthetic construct
GACHATGDLKGTPRLKLRSCHARRSKDH

SEQ ID NO: 734 - protein, 37 aa, Lepidium sativum
RVCLRRSKTWTGFCGNTRGFDSQCKRWECALHGACHC

SEQ ID NO: 735 - protein, 26 aa, synthetic construct
GFCGNTRGFDSQCKRWECALHGACHC

SEQ ID NO: 736 - protein, 18 aa, synthetic construct
GFCGNTRGFDSQCKRWEC

SEQ ID NO: 737-740 - skipped (4 entries)

SEQ ID NO: 741 - protein, 47 aa, synthetic construct
  VARIANT 41..43: note = W, F, or Y
RTCESQSHKFKGPCASDHNCASVCQTERFSGGRCKGFRRRXXXTRIC

SEQ ID NO: 742 - protein, 47 aa, synthetic construct
RTCESQSHKFKGPCASDHNCASVCQTERFSGGRCKGFRRRWFWTRIC

SEQ ID NO: 743 - protein, 53 aa, Olea europaea
KPCTKLSKGWRGLCAPHKCSSYCIHHEGAYHGACLKNRHSKHYGCYCYYRHCY

SEQ ID NO: 744 - protein, 53 aa, synthetic construct
  VARIANT 45..47: note = W, F, or Y
KPCTKLSKGWRGLCAPHKCSSYCIHHEGAYHGACLKNRHSKHYGXXXYYRHCY

SEQ ID NO: 745 - protein, 53 aa, synthetic construct
KPCTKLSKGWRGLCAPHKCSSYCIHHEGAYHGACLKNRHSKHYGWFWYYRHCY

SEQ ID NO: 746 - protein, 45 aa, Medicago truncatula
KTCENLADKYRGPCFSGCDTHCTTKENAVSGRCRDDFRCWCTKNC

SEQ ID NO: 747 - protein, 45 aa, synthetic construct
  VARIANT 39..41: note = W, F, or Y
KTCENLADKYRGPCFSGCDTHCTTKENAVSGRCRILFRXXXTKNC

SEQ ID NO: 748 - protein, 45 aa, synthetic construct
KTCENLADKYRGPCFSGCDTHCTTKENAVSGRCRILFRWFWTKNC

SEQ ID NO: 749 - protein, 50 aa, Medicago truncatula
KLCQKRSTTWSGPCLNTGNCKRQCINVEHATFGACHRQGFGFACFCYKKC

SEQ ID NO: 750 - protein, 50 aa, synthetic construct
  VARIANT 44..46: note = W, F, or Y
KLCQKRSTTWSGPCLNTGNCKRQCINVEHATFGACHRQGFGFAXXXYKKC

SEQ ID NO: 751 - protein, 50 aa, synthetic construct
KLCQKRSTTWSGPCLNTGNCKRQCINVEHATFGACHRQGFGFAWFWYKKC

SEQ ID NO: 752 - protein, 49 aa, Sorghum bicolor
VHVCTMRNKFFHGPCMSNKNCAASCIQHRIGGGGYCSSRRQICKCTLQC

SEQ ID NO: 753 - protein, 49 aa, synthetic construct
  VARIANT 43..45: note = W, F, or Y
VHVCTMRNKFFHGPCMSNKNCAASCIQHRIGGGGYCSSRRQIXXXTLQC

SEQ ID NO: 754 - protein, 49 aa, synthetic construct
VHVCTMRNKFFHGPCMSNKNCAASCIQHRIGGGGYCSSRRQIWFWTLQC

SEQ ID NO: 755 - protein, 49 aa, synthetic construct; identical to SEQ ID NO: 753
  VARIANT 43: note = W, F, or Y
  VARIANT 44: note = K or R
  VARIANT 45: note = W, F, or Y

SEQ ID NO: 756 - protein, 49 aa, synthetic construct
VHVCTMRNKFFHGPCMSNKNCAASCIQHRIGGGGYCSSRRQIWKWTLQC

SEQ ID NO: 757 - protein, 17 aa, synthetic construct
  MOD_RES 17: note = AMIDATION
GGRCRGFRRRCFCTTHC

SEQ ID NO: 758 - protein, 17 aa, synthetic construct
  MOD_RES 17: note = AMIDATION
GGRCKGFRRRCFCTRIC

SEQ ID NO: 759 - protein, 17 aa, synthetic construct
  MOD_RES 17: note = AMIDATION
GGRCRGFRRRCFCTRIC

SEQ ID NO: 760 - protein, 17 aa, synthetic construct
  MOD_RES 17: note = AMIDATION
GGRCRGFRRRVFVTRIC

SEQ ID NO: 761 - protein, 17 aa, synthetic construct
  MOD_RES 17: note = AMIDATION
FGRCRGFRRRCFCWRWC

SEQ ID NO: 762 - protein, 17 aa, synthetic construct
FGRCRGFRRRCFCWRWC

SEQ ID NO: 763 - protein, 17 aa, synthetic construct
  MOD_RES 17: note = AMIDATION
  SITE 3: note = diaminobutyric acid
  SITE 5: note = diaminobutyric acid
  SITE 8..10: note = diaminobutyric acid
  SITE 15: note = diaminobutyric acid
FGXCXGFXXXCFCWXWC

SEQ ID NO: 764 - protein, 17 aa, synthetic construct
  SITE 3: note = diaminobutyric acid
  SITE 5: note = diaminobutyric acid
  SITE 8..10: note = diaminobutyric acid
  SITE 15: note = diaminobutyric acid
FGXCXGFXXXCFCWXWC

SEQ ID NO: 765 - protein, 17 aa, synthetic construct
  MOD_RES 17: note = AMIDATION
GGRCKGFRRRWFWTRIC

SEQ ID NO: 766 - protein, 16 aa, synthetic construct
GRCRGFRRRCFCTTHC

SEQ ID NO: 767 - protein, 22 aa, synthetic construct
GACLKNRHSKHYGFFWYYRHCY

SEQ ID NO: 768 - protein, 22 aa, synthetic construct
  MOD_RES 22: note = AMIDATION
GACLKNRHSKHYGCYCYYRHCY

SEQ ID NO: 769 - protein, 22 aa, synthetic construct
  MOD_RES 22: note = AMIDATION
GACLKNRHSKHYGWFWYYRHCY

SEQ ID NO: 770 - protein, 22 aa, synthetic construct
  MOD_RES 22: note = AMIDATION
GACLKNRHSKHYGFFWYYRHCY

SEQ ID NO: 771 - protein, 16 aa, synthetic construct
  MOD_RES 16: note = AMIDATION
GRCRGFRRRCFCTTHC

SEQ ID NO: 772 - protein, 17 aa, synthetic construct
  MOD_RES 17: note = AMIDATION
GGRCKGFLRRFWFTRIC

SEQ ID NO: 773 - protein, 17 aa, synthetic construct
  MOD_RES 17: note = AMIDATION
GGRCKGFRRRWYWTRIC

SEQ ID NO: 774 - protein, 16 aa, synthetic construct
  MOD_RES 16: note = AMIDATION
SGRCRILFRCFCTKNC

SEQ ID NO: 775 - protein, 16 aa, synthetic construct
  MOD_RES 16: note = AMIDATION
SGRCRILFRWFWTKNC

SEQ ID NO: 776 - protein, 16 aa, synthetic construct
  MOD_RES 16: note = AMIDATION
SGRCRILFRWYFTKNC

SEQ ID NO: 777 - protein, 18 aa, synthetic construct
  MOD_RES 18: note = AMIDATION
GACHRQGFGFACFCYKKC

SEQ ID NO: 778 - protein, 18 aa, synthetic construct
  MOD_RES 18: note = AMIDATION
GACHRQGFGFAWFWYKKC

SEQ ID NO: 779 - protein, 18 aa, synthetic construct
  MOD_RES 18: note = AMIDATION
GACHRQGFGFAFFFYKKC

SEQ ID NO: 780 - DNA, 174 bp, synthetic construct
  source 1..174: mol_type = other DNA
ctcgagaaaagagctaggatgtgcaaaacgccctcaggtaaattcaagggctactgcgtatcctctaccaattgtaagaacgtgtgccgcactgagggatttccgacagggagttgtgattttcacgttgcttcgcggaagtgttactgttataaaccttgctagtaatctaga

SEQ ID NO: 781 - DNA, 177 bp, synthetic construct
  source 1..177: mol_type = other DNA
ctcgagaaaagagctagaacttgtctgtctaagagccacaggtttcacggctattgtgtccgcagttcgtcatgtaaagagatttgccagaccgaacgattcctatccggggaatgtcggcatacacatcttggtaaccggcgttgcttttgcgagaagccttgctagtaatctaga

SEQ ID NO: 782 - DNA, 168 bp, synthetic construct
  source 1..168: mol_type = other DNA
ctcgagaaaagagctcggatgtgcgaaagccagtcgcataagttccacggtctgtgtatgtctgatcgaacctgcgtggtcgtatgcaaagaagaggattttgacgacggcaaatgcaggggattccgtcacagatgtttttgtcgcaagccttgttagtaatctaga

SEQ ID NO: 783 - DNA, 168 bp, synthetic construct
  source 1..168: mol_type = other DNA
ctcgagaaaagagctaaaatctgtgagtacaagagcgaaaggttttggggggtttgtctctctgacataaaatgcgcggatgtgtgtaaggacgaatcgttcgagtctggtgattgccagggtttgcgacgccggtgtatgtgctataatcattgctagtaatctaga

SEQ ID NO: 784 - DNA, 174 bp, synthetic construct
  source 1..174: mol_type = other DNA
ctcgagaaaagagctaaattgtgcgaaaaggcctctctcacttggtcaggtaaatgcgggaatacacagaattgtgataagcaatgtcaaaactgggagagtgcaaaacacggcgcttgccataagcggccaacgtggaagtgcttttgttacagcgactgttagtaatctaga

SEQ ID NO: 785 - DNA, 162 bp, synthetic construct
  source 1..162: mol_type = other DNA
ctcgagaaaagagctagtctgtgcgagaccccttctcgatattttaacgggatttgcttttccaggagaaattgtggagcgatctgtgaaaaagaggggttctcgtcaggccggtgcaaggctttcaaatgcgtttgtggtaaggattgttagtaatctaga

SEQ ID NO: 786 - DNA, 177 bp, synthetic construct
  source 1..177: mol_type = other DNA
ctcgagaaaagagctaagttatgccaaaggccatcgggcacctggtcaggagtttgtggtaataataacgcatgtaaaaaccagtgcattcggttagaaaaagctcgacacgggagttgtaattatgtgttccccgcccataaatgcatctgttactttccgtgctagtaatctaga

SEQ ID NO: 787 - DNA, 183 bp, synthetic construct
  source 1..183: mol_type = other DNA
ctcgagaaaagagctaagcattgcgcgaaactgagtaaggggtggcacggtttttgtgctcctcataaatgcagcaggtactgcatacaccacgagggagcctatgacggggcatgtcttaaacatcatcactcgaagcatattggctgttactgctattaccggcactgttagtaatctaga

SEQ ID NO: 788 - DNA, 171 bp, synthetic construct
  source 1..171: mol_type = other DNA
ctcgagaaaagagctaaattatgtcgaaagccttcgggcacctggtcaggtctttgcgctaatagcgactcttgtagaaagcaatgtattaacctggagaaggcaaaggatggatcctgcaaatataaatttcccgcccacaaatgcttctgttactactagtaatctaga

SEQ ID NO: 789 - DNA, 168 bp, synthetic construct
  source 1..168: mol_type = other DNA
ctcgagaaaagagctcgggtctgccgacgcagaagtgccgggttcaggggactctgtgtaagcaaccataactgcgcgcaagtttgtatgcaggagggctggggtggtgggaattgtgacggatttcgacgtcaatgtaaatgcatgcgccagtgctagtaatctaga

SEQ ID NO: 790 - DNA, 219 bp, synthetic construct
  source 1..219: mol_type = other DNA
ctcgagaaaagagcttacatgtgcaccacacaaaatcgcttcttccatggacgctgcgtcaataacttgaactgtgcgtcgagctgcgttcacggtcgtttaggcactggggggcattgcgccacgagaaaacggtccgcagctgaccctggagtagatatagattgggtttggaataggcgaatttgtgtgtgtgtgtttcagtgttagtaatctaga

SEQ ID NO: 791 - DNA, 171 bp, synthetic construct
  source 1..171: mol_type = other DNA
ctcgagaaaagagctcgaaagtgcgaatcaaaacgtagatggatttacggaaactgcctatccgacgagaagtgtgcctctgattgcctggttaagggctttcttttcggtaaatgtcgggggtacgataatcgctgttattgcaagaaaccaaggtgttagtaatctaga

SEQ ID NO: 792 - DNA, 171 bp, synthetic construct
  source 1..171: mol_type = other DNA
ctcgagaaaagagctaagatttgtgaagttccttcaaaaacatttaagggactttgcgttaaagatcgcaattgcgtcgtgcggtgccatagcgaaggctatggtaacgggaaatgctcgcacgtcagacgtcagtgtatgtgtctcaagatctgtgactagtaatctaga

SEQ ID NO: 793 - DNA, 171 bp, synthetic construct
  source 1..171: mol_type = other DNA
ctcgagaaaagagctaagctttgtcaatatcactcagagacttttcacggagtgtgtgttaccggtaacaattgcaatcgtcattgtcagcgcgaaaacttcgagggtgggcggtgccatggcataagacactacaggtgcgtctgctaccgaacatgttagtaatctaga

SEQ ID NO: 794 - DNA, 168 bp, synthetic construct
  source 1..168: mol_type = other DNA
ctcgagaaaagagctcggcattgcgaatctccatcgcaccgattcaaagggatttgcgttcgtaagtcaaactgtgcggctatatgtcaaaccgagggtttcagcagtggcaaatgtaggggatttagatcccgctgtgtatgcatcaagcactgctagtaatctaga

SEQ ID NO: 795 - DNA, 180 bp, synthetic construct
  source 1..180: mol_type = other DNA
ctcgagaaaagagctcgcatgtgtgagtctaaatcccatgaatttaaaggcttctgcaccaagtggagaatatgccgagatgtttgcattactgagggctttacagacgggggatgtcacggtgtattgcggcattgtgtgtgtaaaaagccttgcttactttcgaactagtaatctaga

SEQ ID NO: 796 - DNA, 222 bp, synthetic construct
  source 1..222: mol_type = other DNA
ctcgagaaaagagctggggcatgtcctctcgtctcaaagcgatctaagacttggtcgggattctgtggtagctccaataattgcaacaaacaatgcaggtcctgggaaagtgcgcagcacggcgcttgtcacaacacgtataagaccgttctgggagtgaaagtaatttctgggcgcgcctgtttctgctacttttgcaaaaagcatacatagtaatctaga

SEQ ID NO: 797 - DNA, 219 bp, synthetic construct
  source 1..219: mol_type = other DNA
ctcgagaaaagagctgttacgtgcccgctggagtccaagcgctcgaaaacatggtcaggagtctgtattaaatctaaaaactgtgataagcagtgtaagacttgggaaagggcaaagcacggcgcctgccatgctacctatcggaatgtattaggtgtgaaagtctttaaagggttcgcgtgcttctgttacttttgcaaaaagcactagtaatctaga

SEQ ID NO: 798 - DNA, 177 bp, synthetic construct
  source 1..177: mol_type = other DNA
ctcgagaaaagagctaagttatgtcaaaaacggtccaaaacctggacagggccctgcatcaaaactaagaattgcgaccatcagtgcaggaagtgggaaaaagctcagcacggcgcctgtcattggcaatggcctggatttgcgtgcttctgttatgtcaactgttagtaatctaga

SEQ ID NO: 799 - DNA, 174 bp, synthetic construct
  source 1..174: mol_type = other DNA
ctcgagaaaagagctcaacgtggttgcaagcaggccagtaaatcaatgaccacgtgtacagcacatccagagtgtgacaaaaagtgcaaaggagaaaaagctaagatggggctctgtcgggcggatggcccgaaagggaagaagtgttggtgctacatgtgctagtaatctaga

SEQ ID NO: 800 - DNA, 183 bp, synthetic construct
  source 1..183: mol_type = other DNA
ctcgagaaaagagctaaattatgccgtcggcgatcaaagacattttctagttcgtgctttatcagcgaacattgcgataaggaatgtaaagagaaggagggcgccaaaaggggaatgtgtattaagaaatccatattcggtcgcttctgttactgctttcacaagtgtaaatagtaatctaga

SEQ ID NO: 801 - DNA, 174 bp, synthetic construct
  source 1..174: mol_type = other DNA
ctcgagaaaagagctgcggtgtgcagtaagccatctacgtatttctttgggccttgcgttcgtcgtagcacctgccgtagagcatgttcacacgagaactaccccgatggcaagtgctcgcgcttcctcggaaaatgtatttgttataaaccgtgtactacatagtaatctaga

SEQ ID NO: 802 - DNA, 165 bp, synthetic construct
  source 1..165: mol_type = other DNA
ctcgagaaaagagctgcgacatgcagaaagccatctatgtacttcagcggcgcatgttttagtcggactaactgtcagaaagcctgtaatcgtgaagattggccgaacggaaaatgtttagtgggtttcaagtgcttttgccaacgcccctgctagtaatctaga

SEQ ID NO: 803 - DNA, 165 bp, synthetic construct
  source 1..165: mol_type = other DNA
ctcgagaaaagagctgcgatttgtaagaagccttcgaagtttttcaaaggagcctgcggtagggacgctgactgcgagaaagcatgtgatcaagagaactggccggggggcgtatgtgtcccattcttgcgctgtgaatgccagcgttcttgctagtaatctaga

SEQ ID NO: 804 - DNA, 174 bp, synthetic construct
  source 1..174: mol_type = other DNA
ctcgagaaaagagctgccacgtgtgctaaaccttccaagtactacaaaggcgggtgctttgcaacaatctctgtgagcgcttgtaaaaagacctgttctcgtgaaaactggccggatggtgcgtgcgtttatccattccgatgcgagtgtcgtcgcccctgttagtaatctaga

SEQ ID NO: 805 - DNA, 174 bp, synthetic construct
  source 1..174: mol_type = other DNA
ctcgagaaaagagctgcaaattgcgctaagccgtcgaaattttaccgtggcccatgcttcaactccatctcagttaaaccgtgtactcagaggtgcagccaggagaactggcccattggggtatgtaccaaggaccttcgctgcgaatgtcaacggccttgttagtaatctaga

SEQ ID NO: 806 - DNA, 168 bp, synthetic construct
  source 1..168: mol_type = other DNA
ctcgagaaaagagctagggtttgtgaagtaccgtccaagacattcacgggtctatgttttagagacacccactgtatcgtccggtgccattctgaggggtacggctatggaaagtgcagtcacgtgcttcgtcgctgccgatgtctgaaagcctgctagtaatctaga

SEQ ID NO: 807 - DNA, 171 bp, synthetic construct
  source 1..171: mol_type = other DNA
ctcgagaaaagagctaaaacctgtgaatcaccctccgcgaagttcgagggcccttgcatcatgggcagtaactgcgccctaacttgccagaaagaaggacatgatggtgggttctgtagagacttttggctcccgaaatgtttttgcacgaagccatgttagtaatctaga

SEQ ID NO: 808 - DNA, 168 bp, synthetic construct
  source 1..168: mol_type = other DNA
ctcgagaaaagagctataacgtgcaagacacaatcgatcaagtacgaaggaatgtgctccagagagaagccatgcgccagtctttgtcagaccaaagggtactctcgtggtcgctgtcatggcgtctataaaatttgtgtatgcgcaactcagtgttagtaatctaga

SEQ ID NO: 809 - DNA, 171 bp, synthetic construct
  source 1..171: mol_type = other DNA
ctcgagaaaagagcttcaatatgtgagattccatccaaaaagtttaaagggctgtgcatacgagatcgtaaatgtattgttatctgccattcggaaggctacgggtatggtaagtgcagccacatcggacgcagatgcatgtgtctcaagacatgtgactagtaatctaga

SEQ ID NO: 810 - DNA, 168 bp, synthetic construct
  source 1..168: mol_type = other DNA
ctcgagaaaagagctaaggattgcctgacccgtaggcacgggttccagggtcggtgtttgttcgaccgacaatgcgcgcatgtatgtcggtccgacggatttatcggcgggcaatgtcgtggacctctaagaaaatgcttttgttctcgcccgtgctagtaatctaga

SEQ ID NO: 811 - DNA, 171 bp, synthetic construct
  source 1..171: mol_type = other DNA
ctcgagaaaagagctcgcatttgccagtcgagaagtactgagtacaggggcatgtgcctgtttgatgtgaactgcgacaatatatgtaaaacagaacctggttttaccggagggcattgtcactccttcttccgatcttgttattgcacgaagccgtgttagtaatctaga

SEQ ID NO: 812 - DNA, 192 bp, synthetic construct
  source 1..192: mol_type = other DNA
ctcgagaaaagagctggccccacaatcgttggaggtaggaaatgtaagtcaccttccaagaaattcaacggtatgtgcatgaatcatgtgaattgtgcaacggtctgtcaaactgaatcgcacgaagatggcaagtgcgaggggttttaccggcgttgtatttgcataaaaccgtgcttttagtaatctaga

SEQ ID NO: 813 - DNA, 171 bp, synthetic construct
  source 1..171: mol_type = other DNA
ctcgagaaaagagctcggacctgtaagtcaaagagtcagagatacaaaggatcctgttattctgacaacaattgcgcaattatctgtgagggcgaagggttcactgggggcaaatgtcatggtttcttttgcaaatgttggtgcaagcgaccatgcctatagtaatctaga

SEQ ID NO: 814 - DNA, 171 bp, synthetic construct
  source 1..171: mol_type = other DNA
ctcgagaaaagagctaataactgtaagtatgaatccaataagtttcatggggtttgcctaagtgacacaaactgtgagtcggtgtgtcagactgaaggattcccgttcggtaagtgcgatggcgtcctcccacgcacctgtgtatgcaaaaaaccttgctagtaatctaga

SEQ ID NO: 815 - DNA, 153 bp, synthetic construct
  source 1..153: mol_type = other DNA
ctcgagaaaagagctcatacctgtcacaaaaagatcgatattaaaacatgcgatttccaaaaatgtaacaaggaatgtgctaaggagacgcttggcgtcggggactgcagaaatgccctatgtttttgcacttactattgctagtaatctaga

SEQ ID NO: 816 - DNA, 171 bp, synthetic construct
  source 1..171: mol_type = other DNA
ctcgagaaaagagctcgaatctgcgtaaaacctgaaccgttggctcactgcacagagacaatttgtacccagttctgcataagaaagtatggtccaccccccgggactgtgggcaaatgcaatctgccgcctggattttgtacgtgttaccataactgttagtaatctaga

SEQ ID NO: 817 - DNA, 168 bp, synthetic construct
  source 1..168: mol_type = other DNA
ctcgagaaaagagctaaaatttgcgagttcccatctacgcttttcaaaggattgtgctttagctcaaataactgtaagcatacatgccgaaaggaacaatttactagagggaagtgttccttactcacacggacgtgtatgtgtaccaaaaaatgctagtaatctaga

SEQ ID NO: 818 - DNA, 174 bp, synthetic construct
  source 1..174: mol_type = other DNA
ctcgagaaaagagctcgccattgtctttcccagtctcacaccttcaagggactgtgctttagttcagaaaactgcgcctcggtttgtaaatcggagaaatttcccggtggccagtgtcaaatgcacggggcgagccggaagtgtttctgcaaagtagtgtgctagtaatctaga

SEQ ID NO: 819 - DNA, 168 bp, synthetic construct
  source 1..168: mol_type = other DNA
ctcgagaaaagagctgatcggtgtgaaacgcactctaggacgtataaaggcagatgcaacaatcataactgctggtcaatctgcattaccgaggggaatactgggggattctgcaagggtacactcttgctgaagtgtatgtgtactagcgagtgttagtaatctaga

SEQ ID NO: 820 - DNA, 174 bp, synthetic construct
  source 1..174: mol_type = other DNA
ctcgagaaaagagctcgacattgtctatcgcagtcacaccacttcaaaggactttgtttaagctcttccaattgtgctaacgtgtgccgcgtcgaacggttccctgatggggagtgccaaaccacggccggcactagaaagtgcttttgcaagcgtatatgttagtaatctaga

SEQ ID NO: 821 - DNA, 177 bp, synthetic construct
  source 1..177: mol_type = other DNA
ctcgagaaaagagctaggcgttgcgtgtctcagtcccacaagttcgttggtagctgtatgcggaaatcgaattgccaacatgtatgccagaccgaagggtttccgtggggcgagtgtagattccatggagggctcttacgccgatgtttttgcaacaaactgtgttagtaatctaga

SEQ ID NO: 822 - DNA, 168 bp, synthetic construct
  source 1..168: mol_type = other DNA
ctcgagaaaagagctcggaaatgcgagagtaagtcacaacagttcaagggtctctgctttataaaagccaattgtgctgcagtctgccgcacggaagggttccactccggccactgccgtggatttcgaaggagatgttactgtaccaaacattgttagtaatctaga

SEQ ID NO: 823 - DNA, 171 bp, synthetic construct
  source 1..171: mol_type = other DNA
ctcgagaaaagagctaggatatgcgaagtaccaagtaagacgttcatcggactttgttttaaagatacaaactgcattgttaggtgtcgctcggaggggtacggctatggtaagtgctcccacttccgtagacggtgccgatgttttaaaccttgtccgtagtaatctaga

SEQ ID NO: 824 - DNA, 198 bp, synthetic construct
  source 1..198: mol_type = other DNA
ctcgagaaaagagctgtaatttgtccgctcgaaagtaaaaggtcgaaaacatggtcaggggtttgtgcccaatccaagaattgcgacaagcagtgcaagacctgggagcgagctaagcacggggcatgccatgcgacgtatggcaaagtgttgggtgtcaaggcttgtatgcacgcgggaaacaaatagtaatctaga

SEQ ID NO: 825 - DNA, 195 bp, synthetic construct
  source 1..195: mol_type = other DNA
ctcgagaaaagagctaagttatgcgagcgacggagtaaaacgtggaccggatggtgcgccaaaacatctcactgtaacaaacagtgtaaggattgggagagagtggagcatggcgcttgtcacggggaatttcttgaaaagccagtttccgcgatctcaaatgcacattgcaagataagcttctagtaatctaga

SEQ ID NO: 826 - DNA, 216 bp, synthetic construct
  source 1..216: mol_type = other DNA
ctcgagaaaagagctggcgcctgtccattggtgcaaaaacgaagtaagacctggagcggattttgtgggtcctcaaataactgcgataaacagtgtcgtacttgggaaaaggctaagcatggtgcttgccatgcaacaggtgacctaaaggggacgccgaggctcaaactgagatcgtgccacgcgcgccggtctaaagaccactagtaatctaga

SEQ ID NO: 827 - DNA, 138 bp, synthetic construct
  source 1..138: mol_type = other DNA
ctcgagaaaagagctcgcgtgtgcttacgtcgatccaagacgtggacagggttttgcggcaataccagaggattcgacagtcaatgtaaacggtgggagtgtgcccttcacggtgcttgccattgttagtaatctaga

SEQ ID NO: 828 - protein, 21 aa, synthetic construct
GVCAVIRARPLFMRWLWKKKC

SEQ ID NO: 829 - protein, 21 aa, synthetic construct
GVCAVIRARPLFMRYLYKKKC

SEQ ID NO: 830 - protein, 21 aa, synthetic construct
GVCAVIRARPLFMRFLFKKKC

SEQ ID NO: 831 - protein, 16 aa, synthetic construct
GHCRGFWRKWYWTRPC

SEQ ID NO: 832 - protein, 16 aa, synthetic construct
GHCRGFWRKYYYTRPC

SEQ ID NO: 833 - protein, 16 aa, synthetic construct
GHCRGFWRKFYFTRPC

(19) United States
(12) Patent Application Publication
(10) Pub. No.: US 2026/0347421 A1
(43) Pub. Date: Nov. 5, 2026

(54) ANTIMICROBIAL PEPTIDES AND MODIFICATIONS THEREOF

(71) Applicant: Donald Danforth Plant Science Center, St. Louis, MO (US)

(72) Inventors: Kirk Czymmek, St. Louis, MO (US); Dilip M. Shah, Chesterfield, MO (US); Siva L.S. Velivelli, St. Louis, MO (US); Saurav Misra, St. Louis, MO (US); Hui Li, SuZhou (CN); Meenakshi Tetorya, St. Louis, MO (US)

(21) Appl. No.: 19/736,614

(22) Filed: Jul. 9, 2026

Related U.S. Application Data
(63) Continuation of application No. 18/392,130, filed on Dec. 21, 2023.
(60) Provisional application No. 63/476,488, filed on Dec. 21, 2022.
(60) Provisional application No. 63/603,414, filed on Nov. 28, 2023.

(51) Int. Cl. C07K 14/47; C07K 14/415; C12N 1/16; C12N 15/82; C12R 1/72; C12R 1/78; C12R 1/84; C12R 1/85

(52) U.S. Cl. CPC C07K 14/4723; C07K 14/415; C12N 1/16; C12N 15/8282; C12R 2001/72; C12R 2001/78; C12R 2001/84; C12R 2001/85

(57) ABSTRACT

Antimicrobial modified defensin or defensin-like peptides, modified C-terminal fragments of a defensin or defensin-like peptides and nucleic acids encoding the same are disclosed. Compositions comprising the defensin variant peptides and methods of their use to control microbial infections of plants and vertebrate subjects as well as contamination of feedstuffs and foodstuffs are also disclosed.

### CROSS-REFERENCE TO RELATED APPLICATIONS

This patent application is a continuation of U.S. non-provisional patent application Ser. No. 18/392,130, filed Dec. 21, 2023, which claims the benefit of U.S. provisional Patent Application Ser. No. 63/603,414, filed Nov. 28, 2023, and U.S. Patent Application Ser. No. 63/476,488, filed Dec. 21, 2022, which are each incorporated herein by reference in their entireties.

### GOVERNMENT SUPPORT STATEMENT

This invention was made with government support under National Science Foundation EAGER Award Number 1955461 awarded by the National Science Foundation. The government has certain rights in the invention.

### SEQUENCE LISTING

The sequence listing contained in the XML file named “P14299US02,” which is 825,444 bytes as measured in the Windows operating system, and which was created on Apr. 3, 2024 and electronically filed herewith, is incorporated herein by reference in its entirety. The sequence listing contained in the XML file named “P14299US02,” which is 823,641 bytes as measured in the Windows operating system, and which was created on Dec. 19, 2023, is incorporated herein by reference in its entirety.

### BACKGROUND

Protection of agriculturally important crops from pathogenic microbes (e.g., fungi or oomycetes) is crucial for the improvement of crop yields. Microbial infections are a particular problem in damp climates and can become a major concern during crop storage, where such infections can result in spoilage and contamination of food or feed products with microbial toxins. Unfortunately, modern growing methods, harvesting and storage systems can promote plant pathogen infections.

Control of plant pathogens is further complicated by the need to simultaneously control multiple microbes of distinct genera. For example, microbes such as Alternaria; Ascochyta; Aphenomyces; Botrytis; Cercospora; Colletotrichum; Diplodia; Erysiphe; Fusarium; Gaeumanomyces; Helminthosporium; Leptosphaeria, Macrophomina; Magnaporthe; Nectria; Peronospora; Phoma; Phakopsora, Phymatotrichum; Phytophthora; Plasmopara; Podosphaera; Puccinia; Pythium; Pyrenophora; Pyricularia; Rhizoctonia; Sclerotium; Sclerotinia; Septoria; Thielaviopsis; Uncinula; Venturia; and Verticillium species are all recognized plant pathogens.

Certain microbes (e.g., fungi, including mold, yeast and dimorphic fungi, or oomycetes) can also be pathogenic to various vertebrates including humans, livestock, companion animals, fish, and the like. Microbes including dermatophytes, Aspergillus, Candida, Cryptococcus, Coccidiomyces, Penicillium, Rhizopus, Apophysomyces, Cunninghamella, Saksenaea, Rhizomucor, Syncephalostrum, Cokeromyces, Actinomucor, Pythium, Fusarium, Histoplasmosis, or Blastomyces species are also important vertebrate pathogens.

A group of proteins known as defensins have been shown to inhibit plant pathogens. Defensins have been previously identified as small cysteine-rich peptides of about 45-54 amino acids that constitute an important component of the innate immunity of plants (Shafee et al., 2016; Thomma et al., 2002; Lay and Anderson, 2005; Vriens et al., 2014). Widely distributed in plants, defensins vary greatly in their amino acid composition. However, they all have a compact shape which is stabilized by either four or five intramolecular disulfide bonds. Plant defensins have previously been characterized as comprising a conserved gamma-core (i.e., γ-core) peptide comprising a conserved GXCX3-9C (where X is any amino acid) sequence (Sagaram et al., 2011; Lacerda et al., 2014). The three-dimensional structures of previously characterized gamma-core peptides consists of two antiparallel β-sheets, with an interpolated turn region (Ibid.). Antimicrobial activity of certain defensins has been correlated with the presence of positively charged amino acid residues in the gamma-core peptide (Spelbrink et al, Plant Physiol., 2004; Sagaram et al, 2013).

Plant defensins have been extensively studied for their role in plant defense. Some plant defensins at micromolar concentrations inhibit the growth of a broad range of microbes (Broekaert et al, 1995; Broekaert et al, 1997; da Silva Conceicao and Broekaert, 1999). When expressed in transgenic plants, these confer strong resistance to microbial pathogens (da Silva Conceicao and Broekaert, 1999; Thomma et al., 2002; Lay and Anderson, 2005). Two small cysteine-rich proteins isolated from radish seed, Rs-AFP1 and Rs-AFP2, inhibited the growth of many pathogenic microbes when the pure proteins were added to an in vitro antimicrobial assay medium (U.S. Pat. No. 5,538,525). Transgenic tobacco plants containing the gene encoding Rs-AFP2 protein were found to be more resistant to attack by microbes than non-transformed plants.

Defensin genes have also been identified in the legume Medicago truncatula (Hanks et al., 2005). The cloned M. truncatula defensin protein MtDef2 has been demonstrated through in vitro experiments to have little or no antimicrobial activity (Spelbrink et al., 2004). In contrast, the Medicago truncatula defensin proteins MtDef4 (U.S. Pat. No. 7,825,297; incorporated herein by reference in its entirety) and MtDef5 (WO2014179260 and US Patent Appl. Pub. No. 20160208278; both incorporated herein by reference in its entirety) have antimicrobial activity. The peptide GMA4-C, which consists of the C-terminal 16 amino acids of the MtDef4 defensin protein inhibits Fusarium graminearum at concentrations as low as 3 μM in vitro (Sagaram et al., 2011)

Plant defensins with potent in vitro antifungal activity in vitro often fail to confer effective disease resistance in planta. This constrains their commercial development as antifungal agents in transgenic crops. Antifungal plant defensins are generally cationic and cationic residues in their sequences are believed to initiate passage through fungal cell envelopes by electrostatic interactions with anionic fungal cell membranes (Kerenga et al., 2019). Potassium (K+) is an essential macronutrient and is also the most abundant cation in plants. The concentration of K+ in the plant cell cytoplasm is consistently between 100 and 200 mM (Shabala and Pottosin, 2010 and between 10 and 200 mM in the apoplast (White and Karley, 2010). Calcium is an essential secondary micronutrient and its concentrations can range from 0.1% to 6% of the dry weight of plants (Broadley et al., 2003). The concentrations of sodium (Na+) in plants range from 0.001%-8% (Marschner, 1995). Na+ is an essential micronutrient for plants in saline soils.

Many plant defensins that have been characterized to date lose their antifungal activity at elevated concentrations of mono- and bivalent cations such as 100 mM KCl or 2 mM CaCl₂). However, the maize (Zea mays) defensin ZmD32, which has a predicted charge of +10.1 at pH 7 exhibits inhibitory activity against Candida sp. and E. coli in the presence of 100 mM NaCl. Similarly, the Nicotiana benthamiana plant defensin NbD6 having a predicted charge of +7.6 at pH 7, exhibits inhibitory activity against Candida albicans in the presence of 100 mM NaCl (Kerenga et al., 2019).

### SUMMARY

Peptides comprising the amino acid sequence of a modified defensin or modified defensin-like peptide wherein the wild-type gamma-core consensus peptide GXCX3-9C or GXCX3-22C of the wild-type defensin or wild-type defensin-like peptide is replaced by a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M) (F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22(F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), and wherein the peptide has a net positive charge of at least 3 and a hydrophobic amino acid content at least 18% are provided. Peptides comprising the amino acid sequence of a modified defensin peptide fragment wherein the wild-type gamma-core consensus peptide of GXCX3-9C or GXCX3-22C of the corresponding wild-type defensin peptide fragment is replaced by a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M)(F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), wherein the peptide further comprises a second C-terminal cysteine residue located C-terminal to the cysteine residue in the modified gamma-core consensus sequence, wherein the peptide has a net positive charge of at least 3 and a hydrophobic amino acid content at least 18%, and optionally wherein the peptide comprises, essentially consists, or consists of: (i) 30 amino acid residues or less; or (ii) 15, 16, or 17 to 30 amino acid residues are provided. Peptides comprising a C-terminal fragment of a defensin-like peptide, wherein said C-terminal fragment lacks 1 to 35 amino terminal amino acids of the corresponding wild-type defensin-like peptide and/or comprises a modified gamma-core consensus peptide comprising the peptide sequence e GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M) (F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M) are provided. Peptides comprising, consisting essentially of, or consisting of SEQ ID NO: 729, 731, 733, 735, 736, 828, 829, 830, 831, 832, or 833 a variant thereof having comprising a conservative amino acid substitution of 1 to 2, 3, 4, or 5 amino acid residues, or a variant thereof having at least 90% or 95% sequence identity thereto, wherein the gamma-core consensus peptide is conserved in the variants or wherein the gamma-core consensus peptide is replaced with a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M) (F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), optionally wherein the gamma-core consensus peptide comprises the peptide sequence GXCX3-9C or GXCX9-16C, are provided. In certain embodiments, the aforementioned peptide exhibits antimicrobial activity, wherein the antimicrobial activity is optionally one or more of an antifungal or antibacterial activity. In certain embodiments, the peptide is an isolated peptide. Compositions comprising any of the aforementioned peptides and an agriculturally, pharmaceutically, or veterinary-practicably acceptable carrier, diluent, or excipient are also provided. Compositions comprising any of the aforementioned peptides and carriers, diluents, or excipients for use in treating, preventing, or inhibiting microbial infection in a subject in need thereof are also provided.

Methods for: (i) preventing or reducing crop damage by a plant pathogenic microbe or (ii) preventing contamination of plants, plant parts, seeds, feedstuff obtained therefrom, or foodstuff obtained therefrom with an undesirable microbe, comprising the step of contacting a plant, a plant seed, or other part of said plant with an effective amount of any of the aforementioned compositions, where the composition optionally comprises an agriculturally acceptable carrier, diluent, or excipient, are also provided.

Plant parts including seeds which are least partly coated with any of the aforementioned compositions, where the composition optionally comprises an agriculturally acceptable carrier, diluent, or excipient, are also provided.

Methods for treating, preventing, or inhibiting a microbial infection in a subject in need thereof comprising administering to said subject an effective amount of the aforementioned compositions are provided.

Medical devices comprising the device and an aforementioned composition, wherein the device comprises at least one surface that is topically coated and/or impregnated with the composition, where the composition optionally comprises a pharmaceutically or veterinary-practicably acceptable carrier, diluent, or excipient are also provided.

Methods for treating, preventing, or inhibiting a microbial infection in a subject in need thereof comprising administering to said subject an effective amount of any of the aforementioned compositions, where the composition optionally comprises a pharmaceutically or veterinary-practicably acceptable carrier, diluent, or excipient are also provided. Use of any of any of the aforementioned compositions in a method of treating, preventing, or inhibiting microbial or yeast infection in a subject in need thereof are provided. Use of any of the aforementioned first antimicrobial peptide or proteins in the manufacture of a medicament or composition for inhibiting microbial or yeast infection in a subject in need thereof are also provided.

Recombinant polynucleotides comprising a polynucleotide encoding a peptide comprising any of the aforementioned peptides, wherein the polynucleotide encoding the antimicrobial peptide is operably linked to a polynucleotide comprising a promoter which is heterologous to the polynucleotide encoding the peptide are provided. Recombinant polynucleotides encoding a peptide comprising: (i) a defensin peptide comprising a long gamma-core consensus sequence GXCX16-22C, optionally wherein the defensin peptide comprises SEQ ID NO: 578, 608, 612, or a variant thereof having at least 70%, 75%, 80%, 85%, 90%, 95%, 98%, or 99% sequence identity to SEQ ID NO: 578, 608, or 612, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues; or (ii) a defensin or defensin-like peptide comprising a wild-type gamma-core consensus peptide GXCX₃₋₉C, GXCX₉₋₁₆C or GXCX₃₋₂₂C, optionally wherein the defensin-like peptide comprises SEQ ID NO: 534, 538, 560, 565, 569, 573, 583, 598, 603, 616, 624, 628, 633, 645, 697, 702, 711, 715, 723, 728, 729, 730, 731, 732, 733, 734, 735, 736, or a variant thereof having at least 70%, 75%, 80%, 85%, 90%, 95%, 98%, or 99% sequence identity to SEQ ID NO: 534, 538, 560, 565, 569, 573, 583, 598, 603, 616, 624, 628, 633, 645, 697, 702, 711, 715, 723, 728, 729, 730, 731, 732, 733, 734, 735, 736, a variant thereof comprising a deletion of 1 to 10 or 1 to 35 N-terminal amino acid residues, a deletion of 1 to 10 C-terminal amino acid residues, and/or a variant thereof having a conservative amino acid substitution of 1 to 10 amino acid residues; wherein the polynucleotide encoding the peptide is operably linked to a polynucleotide comprising a promoter which is heterologous to the polynucleotide encoding the peptide are provided.

Isolated peptide comprising: (i) peptides encoded by any of the aforementioned recombinant polynucleotides; (ii) a defensin peptide comprising a long gamma-core consensus sequence GXCX16-22C, optionally wherein the defensin peptide comprises SEQ ID NO: 578, 608, 612, or a variant thereof having at least 70%, 75%, 80%, 85%, 90%, 95%, 98%, or 99% sequence identity to SEQ ID NO: 578, 608, or 612, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues; or (iii) a defensin or defensin-like peptide comprising a wild-type gamma-core consensus peptide GXCX3-9C, GXCX9-16C or GXCX3-22C, optionally wherein the defensin or defensin-like peptide comprises SEQ ID NO: 534, 538, 560, 565, 569, 573, 583, 598, 603, 616, 624, 628, 633, 645, 697, 702, 711, 715, 723, 728, 729, 730, 731, 732, 733, 734, 735, 736, or a variant thereof having at least 70%, 75%, 80%, 85%, 90%, 95%, 98%, or 99% sequence identity to SEQ ID NO: 534, 538, 560, 565, 569, 573, 583, 598, 603, 616, 624, 628, 633, 645, 697, 702, 711, 715, 723, 728, 729, 730, 731, 732, 733, 734, 735, 736, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues are provided. Compositions comprising the aforementioned peptides of embodiment and an agriculturally, pharmaceutically, or veterinary-practicably acceptable carrier, diluent, or excipient are provided.

Plant nuclear or plastid genomes comprising a polynucleotide encoding an antimicrobial peptide comprising any of the aforementioned peptides, wherein the polynucleotide is heterologous to the nuclear or plastid genome and wherein the polynucleotide is operably linked to an endogenous promoter of the nuclear or plastid genome, are provided.

Cells, plants, and plant parts including seeds comprising the aforementioned recombinant polynucleotides or genomes and/or which are at least partially coated with any of the aforementioned compositions are provided. Methods for producing plant seed that provides plants resistant to infection by a plant pathogenic microbe that comprises the steps of: (i) selfing or crossing the aforementioned plant; and (ii) harvesting seed that comprises the recombinant polynucleotide of the plant from the self or cross, thereby producing plant seed that provide plants resistant to infection by a plant pathogenic microbe are provided. Methods for producing antifungal peptides comprising: (i) culturing the cells comprising the aforementioned recombinant polynucleotides under conditions wherein the peptide, defensin, or defensin-like peptide is expressed by the cell; and (ii) purifying the peptide, defensin, or defensin-like peptide from the culture are provided.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a non-limiting subset of reference wild-type defensin peptides aligned at the conserved C1, C2, C3, C4, C5, C6, C7, and C8 cysteines (in bold). The conserved gamma-core peptide is underlined.

FIG. 2 shows a non-limiting subset of reference wild-type defensin peptides aligned at the conserved C1, C2, C3, C4, C5, C6, C7, and C8 cysteines (in bold). The conserved gamma-core peptide is underlined. Beneath each wild-type defensin peptide are non-limiting examples of a modified defensin peptide and a modified defensin C-terminal fragment aligned at the conserved and unsubstituted C₁, C2, C3, C4, C5, and C8 cysteines (in bold), where at least the conserved C6 and C7 cysteine residues are substituted. Substituted residues in the modified defensin peptide and a modified defensin C-terminal fragment are double underlined and shown in italics. The conserved gamma-core peptide is underlined in the wild-type defensin peptides and is absent from the modified gamma-core consensus peptides. The corresponding amino acid residue numbers of the modified defensin peptides that are present in the modified C-terminal fragment peptides are shown in the parentheses.

FIG. 3A, B show preventative (FIG. 3A) and curative (FIG. 3B) effects of antifungal peptide PD122.1.1 (SEQ ID NO: 715 with an N-terminal alanine residue).

FIG. 4 shows curative effects of peptide PD101.1 in wheat plant leaves inoculated with Z. tritici.

### DETAILED DESCRIPTION

### Definitions

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

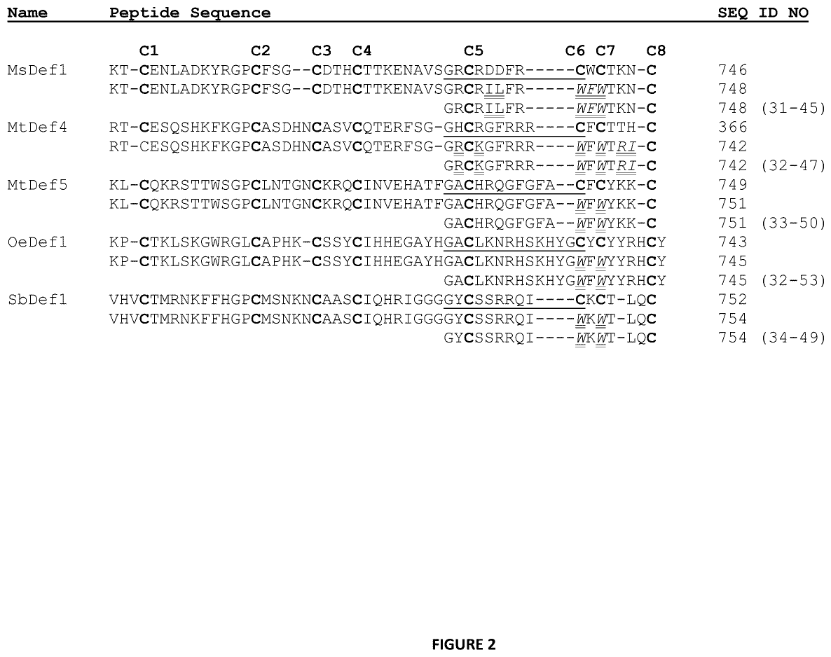

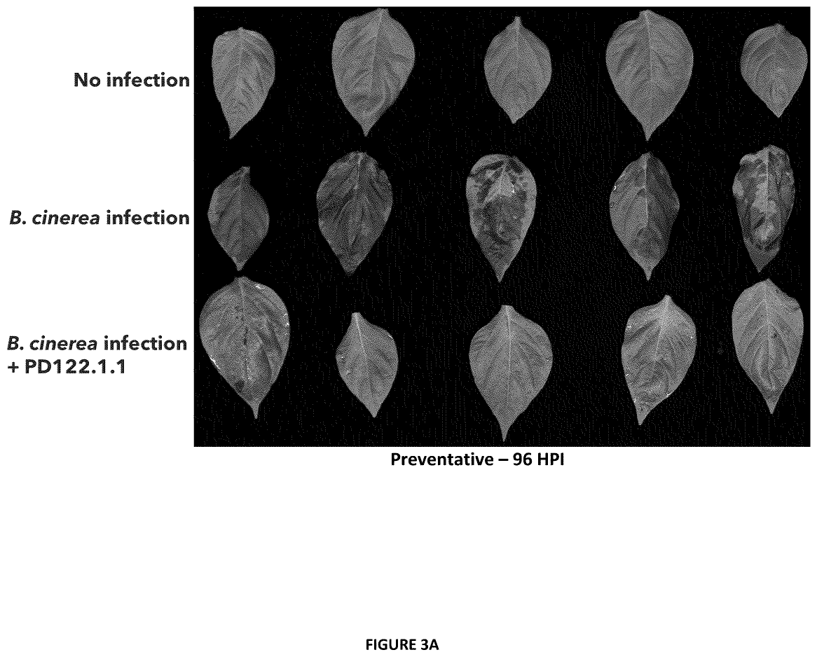

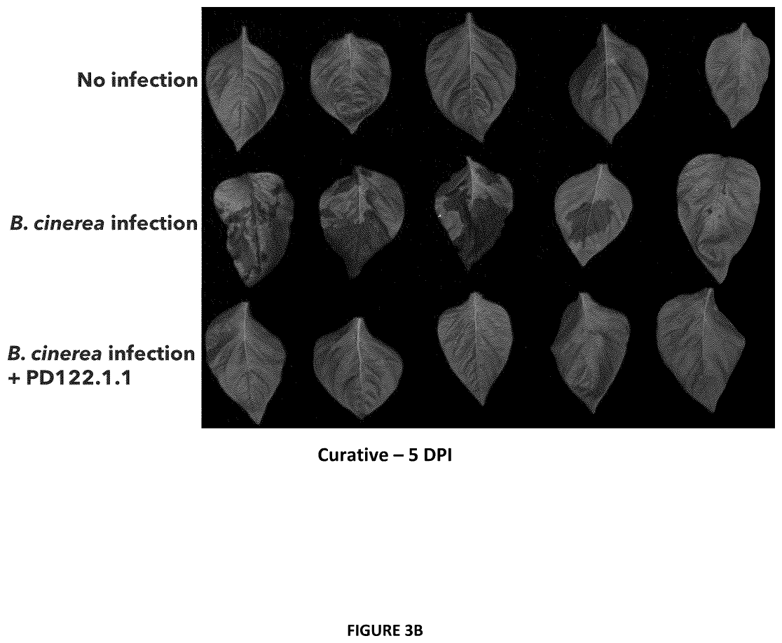

As used herein, the terms “correspond,” “corresponding,” and the like, when used in the context of an amino acid position, mutation, and/or substitution in any given peptide (e.g., a defensin variant peptide) with respect to the reference peptide sequence (e.g., reference defensin C-terminal peptide sequence including SEQ ID NO: 8, 16, 20, 23, 26, 29, 37, 38, 39, 40, 41 or 89-122) all refer to the amino acid residue in the given peptide sequence that has the same location in the given peptide as the residue in the reference amino acid sequence when the given peptide is aligned to the reference sequence. In certain embodiments, the alignment is an alignment of the 4 conserved cysteine residues of a defensin C-terminal peptide of a defensin variant peptide and a reference defensin C-terminal peptide sequence (e.g., as shown in FIGS. 2 and 3).

As used herein, the terms “include,” “includes,” and “including” are to be construed as at least having the features to which they refer while not excluding any additional unspecified features.

Where a term is provided in the singular, other embodiments described by the plural of that term are also provided.

The phrase “antimicrobial peptide” as used herein refer to peptides which exhibit any one or more of the following characteristics of inhibiting the growth of microbial cells, killing microbial cells, disrupting or retarding stages of the microbial life cycle such as spore germination, sporulation, or mating, and/or disrupting microbial cell infection, penetration or spread within a plant or other susceptible subject, including a human, livestock, poultry, fish, or a companion animal (e.g. dog or cat).

As used herein, the terms “acidic” or “anionic” are used interchangeably to refer to amino acids such as aspartic acid and glutamic acid.

As used herein, the term “amino acid” refers to an organic compound that contains amino (—NH₃) and carboxylate (—CO₂) functional groups, along with a side chain (R group) specific to each amino acid. Amino acid residues in polypeptides are in certain instance referred to herein by one letter amino acid codes as follows: G—Glycine (Gly); P—Proline (Pro); A—Alanine (Ala); V—Valine (Val); L—Leucine (Leu); I—Isoleucine (Ile); M—Methionine (Met); C—Cysteine (Cys); F—Phenylalanine (Phe); Y—Tyrosine (Tyr); W—Tryptophan (Trp); H—Histidine (His); K—Lysine (Lys); R—Arginine (Arg); Q—Glutamine (Gln); N—Asparagine (Asn); E—Glutamic Acid (Glu); D—Aspartic Acid (Asp); S—Serine (Ser); or T—Threonine (Thr).

As used herein, the terms “basic” and “cationic” are used interchangeably to refer to amino acids such as arginine, histidine, and lysine.

As used herein, the phrase “cation-tolerant” refers to a defensin peptide or modified defensin or defensin-like peptide which exhibits equivalent in vitro antifungal or antimicrobial activity or no more than about a 1.5-, 2-, 3-, or 4-fold decrease in in vitro antifungal or antimicrobial activity in the presence of 100 mM KCl or 100 mM NaCl as compared to the antifungal activity of the defensin peptide or modified defensin or defensin-like peptide in the absence of KCl or NaCl.

As used herein, the phrase “consensus sequence” refers to an amino acid, DNA or RNA sequence created by aligning two or more homologous sequences and deriving a new sequence having either the conserved or set of alternative amino acid, deoxyribonucleic acid, or ribonucleic acid residues of the homologous sequences at each position in the created sequence.

The phrases “preventing crop damage” and “reducing crop damage” as used herein refer to prevention or reduction in damage to a crop plant or crop plant product due to infection by a microbial pathogen. More generally, these phrases refer to reduction in the adverse effects caused by the presence of a pathogenic microbe in the crop plant. Adverse effects of microbial growth are understood to include any type of plant tissue damage or necrosis, any type of plant yield reduction, any reduction in the value of the crop plant product, and/or production of undesirable microbial metabolites or microbial growth by-products including to mycotoxins.

The phrase “defensin peptide” is used herein to refer to a peptide comprising a conserved gamma-core peptide and two additional cysteine residues located C-terminal to the C-terminal cysteine residue of the conserved gamma-core peptide. Plant defensins have been previously characterized as comprising a conserved GXCX3-9C gamma-core peptide sequence, where X is any amino acid residue (Lacerda et al.) or a conserved GXCX3-10C variant gamma-core peptide sequence, where X is any amino acid residue other than cysteine. In certain embodiments, defensin peptides disclosed herein can also include non-standard defensin gamma-core peptides comprising a GXCX3-12C, GXCX3-15C, GXCX3-22C, or GXCX16-22C. Therefore, as used in this disclosure, a plant defensin or defensin or C-terminal peptide comprising fragment thereof can comprise a conserved GXCX3-9C, GXCX3-12C, GXCX3-15C, GXCX3-22C, or GXCX16-22C gamma-core peptide sequence, where X is any amino acid residue other than cysteine. Defensin peptides include proteins that are antimicrobial, that can permeabilize plasma membranes, that can bind phospholipids, that can bind sphingolipids, or that exhibit any combination of those properties. A defensin peptide can be naturally occurring or non-naturally occurring (e.g., synthetic and/or chimeric).

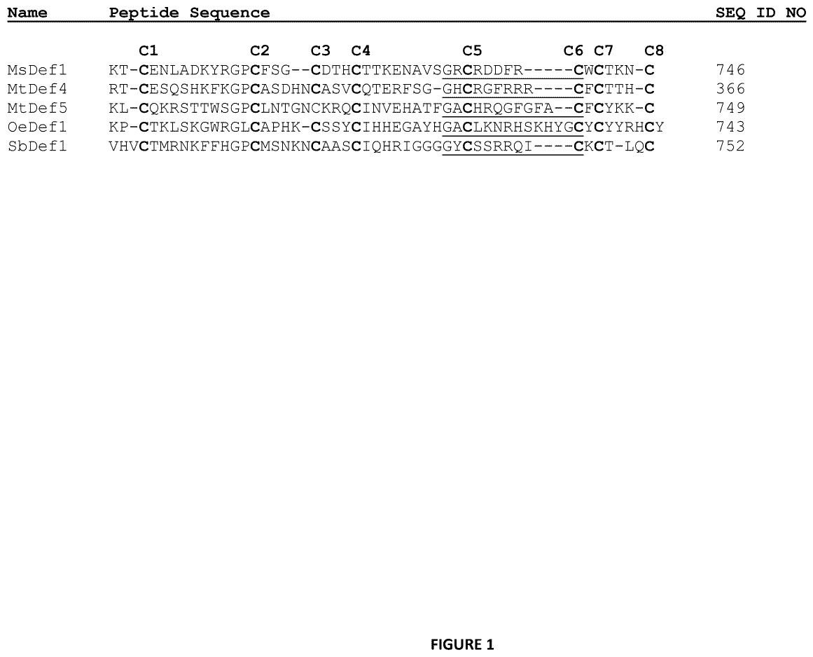

The phrase “defensin-like peptide” is used herein to refer to a peptide comprising a conserved gamma-core peptide but lacking at least one of the two additional cysteine residues located C-terminal to the C-terminal cysteine residue of the conserved gamma-core peptide. In certain embodiments, defensin-like peptides can thus contain 2 or 3 disulfide bonds (as opposed to 4 in a classical C8 defensin as depicted in FIGS. 1 and 2) and can contain a cysteine-stabilized α/β fold. In certain embodiments, defensin-like peptides disclosed herein can also include defensin gamma-core peptides comprising a GXCX3-12C, GXCX3-15C, GXCX9-16C, GXCX3-22C, or GXCX16-22C peptide sequence. Examples of defensin-like peptides disclosed herein include the peptides of SEQ ID NO: 728, 730, 732, 734, and variants thereof having at least 70%, 75%, 80%, 85%, 90%, 95%, 98%, and 99% identity thereto.

As used herein, the term “exemplary” refers to an example, an instance, or an illustration, and does not indicate a most preferred embodiment unless otherwise stated.

The phrases “modified defensin peptide,” “modified defensin-like peptide”, or “modified defensin or defensin-like peptide”, are used herein to describe a variant defensin or defensin-like peptide comprising either: (i) a conserved gamma-core peptide of GXCX3-12C, GXCX3-15C, GXCX3-22C, GXCX9-22C, or GXCX16-22C and at least one amino acid substitution in the corresponding wild-type defensin or defensin-like peptide; (ii) C-terminal fragment of a wild-type defensin or defensin-like peptide comprising a conserved gamma-core peptide of GXCX3-12C, GXCX3-15C, GXCX3-22C, GXCX9-22C, or GXCX16-22C and lacking at least one amino acid residue N-terminal to the conserved gamma-core peptide; or (iii) a modified gamma-core variant sequence. In certain embodiments, modified defensin or defensin-like peptides provided herein are variants of full length defensin peptides wherein the wild-type gamma-core consensus peptide of GXCX₃₋₉C or GXCX₃₋₂₂C of the wild-type defensin or wild-type defensin-like peptide is replaced by a modified gamma-core consensus peptide (e.g., GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M)(F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22(F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), where X is any amino acid other than cysteine). In certain embodiments, modified defensin or defensin-like peptides provided herein are less than full length defensin or defensin-like peptides (e.g., C-terminal fragments of a defensin or defensin-like peptide comprising a gamma-core peptide sequence or a modified gamma-core sequence (e.g., peptides) comprising, consisting essentially of, or consisting of: (i) 30 amino acid residues or less; or (ii) 15, 16, or 17 to 30 amino acid residues).

The phrase “modified defensin C-terminal fragment” is used herein to refer to a fragment of a defensin protein wherein at least one amino acid residue has been deleted from the N-terminus and wherein the wild-type consensus gamma-core sequence has been replaced with a modified gamma-core peptide. In certain embodiments, the modified defensin C-terminal fragments can comprise a substitution of at least the conserved C6 cysteine or at least the conserved C6 and C7 cysteines of a wild-type defensin with a phenylalanine, tryptophan, tyrosine, leucine, valine, isoleucine, or methionine residue. In certain embodiments, the modified defensin C-terminal fragment comprises the modified gamma-core peptide GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M) (F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), where X is any amino acid other than cysteine.

The phrase “reference defensin peptide” or “wild-type defensin” is used herein to refer to a full length defensin peptide comprising a conserved GXCX3-9C or GXCX3-22C gamma-core sequence, and the two additional conserved cysteine residues located C-terminal to the gamma-core peptide sequence, wherein the cysteine located closest to the N-terminus of the reference defensin C-terminal peptide corresponds to the cysteine located closest to the N-terminus of the gamma-core sequence. In certain embodiments, reference defensin C-terminal peptides thus include 8 conserved cysteine residues, which are referred to herein as C1 (most N-terminal in both the peptide and in the gamma-core peptide sequence), C2 (C-terminal to C1), C3 (C-terminal to C2), C4 (C-terminal to C3), C5 (C-terminal to C4 and located in the gamma-core peptide sequence). An alignment of a non-limiting subset of reference defensin C-terminal peptides comprising the conserved gamma-core peptide sequence and the C1, C2, C3, C4, C5, C6, C7, and C8 cysteines is shown in FIG. 1. An alignment of a non-limiting subset of wild-type defensin peptides of SEQ ID NO: 366, 743, 746, 749, and 752 comprising the conserved gamma-core peptide sequence and the C1, C2, C3, C4, C5, C6, C7, and C8 cysteines with exemplary modified defensin peptides and exemplary modified defensin C-terminal fragments where at least the conserved C6 and C7 cysteines are substituted with the indicated amino acids is shown in FIG. 2. Other wild-type defensin peptides include those contained in the full length defensin peptides of SEQ ID NO: 11-41, 81 to 533, 534, 538, 542, 546, 551, 555, 560, 565, 569, 573, 578, 583, 588, 593, 598, 603, 608, 612, 616, 620, 624, 628, 633, 637, 641, 645, 649, 653, 657, 662, 666, 670, 674, 679, 683, 688, 692, 697, 702, 706, 711, 715, 719, 723, 743, 746, 749, or 752.

As used herein, the terms “edit,” “editing,” “edited” and the like refer to processes or products where insertions, deletions, and/or nucleotide substitutions are introduced into a genome. Such processes include methods of inducing homology directed repair and/or non-homologous end joining of one or more sites in the genome.

The phrases “genetically edited plant” or “edited plant” are used herein to refer to a plant comprising one or more nucleotide insertions, deletions, substitutions, or any combination thereof in the genomic DNA of the plant. Such genetically edited plants can be constructed by techniques including CRISPR/Cas endonuclease-mediated editing, meganuclease-mediated editing, engineered zinc finger endonuclease-mediated editing, and the like.

The term “heterologous”, as used herein in the context of a second polynucleotide that is operably linked to a first polynucleotide, refers to: (i) a second polynucleotide that is derived from a source distinct from the source of the first polynucleotide; (ii) a second polynucleotide derived the same source as the first polynucleotide, where the first, second, or both polynucleotide sequence(s) is/are modified from its/their original form; (iii) a second polynucleotide arranged in an order and/or orientation or in a genomic position or environment with respect to the first polynucleotide that is different than the order and/or orientation in or genomic position or environment of the first and second polynucleotides in a naturally occurring cell; or (iv) the second polynucleotide does not occur in a naturally occurring cell that contains the first polynucleotide. Heterologous polynucleotides include polynucleotides that promote transcription (e.g., promoters and enhancer elements), transcript abundance (e.g., introns, 5′UTR, and 3′UTR), translation, or a combination thereof as well as polynucleotides encoding modified defensin or defensin-like peptides or defensin peptides, spacer peptides, or localization peptides. In certain embodiments, a nuclear or plastid genome can comprise the first polynucleotide, where the second polynucleotide is heterologous to the nuclear or plastid genome. A “heterologous” polynucleotide that promotes transcription, transcript abundance, translation, or a combination thereof as well as polynucleotides encoding modified defensin or defensin-like peptides or defensin peptides, spacer peptides, or localization peptides can be autologous to the cell but, however, arranged in an order and/or orientation or in a genomic position or environment that is different than the order and/or orientation in or genomic position or environment in a naturally occurring cell. A polynucleotide that promotes transcription, transcript abundance, translation, or a combination thereof as well as polynucleotides encoding modified defensin or defensin-like peptides or defensin peptides, spacer peptides, or localization can be heterologous to another polynucleotide when the polynucleotides are not operably linked to one another in a naturally occurring cell. Heterologous peptides or proteins include peptides or proteins that are not found in a cell or organism as the cell or organism occurs in nature. As such, heterologous peptides or proteins include peptides or proteins that are localized in a subcellular location, extracellular location, or expressed in a tissue that is distinct from the subcellular location, extracellular location, or tissue where the peptide is found in a cell or organism as it occurs in nature. Heterologous polynucleotides include polynucleotides that are not found in a cell or organism as the cell or organism occurs in nature.

The phrases “inhibiting growth of a plant pathogenic microbe,” “inhibit microbial growth”, and the like as used herein refers to methods that result in any measurable decrease in microbial growth, where microbial growth includes any measurable decrease in the numbers and/or extent of microbial cells, spores, conidia, or mycelia. As used herein, “inhibiting growth of a plant pathogenic microbe” is also understood to include any measurable decrease in the adverse effects cause by microbial growth in a plant. Adverse effects of microbial growth in a plant include any type of plant tissue damage or necrosis, any type of plant yield reduction, any reduction in the value of the crop plant product, and/or production of undesirable microbial metabolites or microbial growth by-products including mycotoxins. As used herein, the phrase “inhibition of microbial growth” and the like, unless otherwise specified, can include inhibition in a plant, human or animal.

The phrases “percent identity” or “sequence identity” as used herein refer to the number of elements (i.e., amino acids or nucleotides) in a sequence that are identical within a defined length of two DNA, RNA segments in an alignment resulting in the maximal number of identical elements and is calculated by dividing the number of identical elements by the total number of elements in the defined length of the aligned segments and multiplying by 100.

The phrase “transgenic” refers to an organism or progeny thereof wherein the organism's or progeny organism's DNA of the nuclear or organellar genome contains an inserted exogenous DNA molecule of 10 or more nucleotides in length. The phrase “transgenic plant” refers to a plant or progeny thereof wherein the plant's or progeny plant's DNA of the nuclear or plastid genome contains an introduced exogenous DNA molecule of 10 or more nucleotides in length. Such introduced exogenous DNA molecules can be naturally occurring, non-naturally occurring (e.g., synthetic and/or chimeric), from a heterologous source, or from an autologous source.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Further Description

Antimicrobial peptides referred to as modified defensin or defensin-like peptides are provided herein. The antimicrobial peptides and proteins can be applied directly to a plant, feedstuffs, or foodstuffs; applied to a plant in the form of microorganisms that produce the modified defensin or defensin-like peptide or protein, or the plants can be genetically edited to produce the modified defensin or defensin-like peptide or protein. The present disclosure also relates to recombinant or edited polynucleotides, microorganisms and plants transformed with the recombinant or edited polynucleotides, plants comprising genetically edited nuclear or plastid genomes encoding the modified defensin or defensin-like peptides and proteins and compositions comprising the modified defensin or defensin-like peptides and proteins useful in controlling pathogenic microbes including plant pathogenic microbes. In certain embodiments, the defensin variant protein comprising two modified defensin or defensin-like peptides or a modified defensin or defensin-like peptide and another peptide (including a modified defensin or defensin-like peptide or defensin peptide) can provide for improved inhibition of microbial growth when compared to a protein containing only one of the antimicrobial peptides found in the defensin variant protein. In certain embodiments, the modified defensin or defensin-like peptides provided herein are cation-tolerant. Such cation-tolerant defensins can be more effective than cation-sensitive defensins in providing effective control of plant pathogenic microbes in transgenic crops. Cation-tolerant defensins provided herein can function (e.g., inhibit plant pathogenic microbes including fungal pathogens) in the normal cation-rich physiological environment of plant tissues. Cation-tolerant defensins provided herein can also function (e.g., inhibit pathogenic microbes including fungal pathogens) in the normal cation-rich physiological environment of a subject (e.g., a human or animal) infected with pathogenic microbes. Also provided herein are recombinant polynucleotides comprising a polynucleotide encoding a peptide comprising a modified defensin, a modified defensin C-terminal fragment, a defensin-like molecule, a modified defensin-like molecule, or a defensin containing a long chain C16-C22 gamma core consensus peptide, operably linked to a polynucleotide comprising a promoter that is heterologous to the polynucleotide encoding the peptide. In certain embodiments, the peptide is a modified defensin, a modified defensin C-terminal fragment, or defensin-like peptide. In certain embodiments, modified defensin peptides and defensin like peptides include the peptides of SEQ ID NO: 535, 536, 537, 539, 540, 541, 543, 544, 545, 547, 548, 549, 550, 552, 553, 554, 556, 557, 558, 559, 561, 562, 563, 564, 566, 567, 568, 570, 571, 572, 574, 575, 576, 577, 579, 580, 581, 582, 584, 585, 586, 587, 589, 590, 591, 592, 594, 595, 596, 597, 599, 600, 601, 602, 604, 605, 606, 607, 609, 610, 611, 613, 614, 615, 617, 618, 619, 621, 622, 623, 625, 626, 627, 629, 630, 631, 632, 634, 635, 636, 638, 639, 640, 642, 643, 644, 646, 647, 648, 650, 651, 652, 654, 655, 656, 658, 659, 660, 661, 663, 664, 665, 667, 668, 669, 671, 672, 673, 675, 676, 677, 678, 680, 681, 682, 684, 685, 686, 687, 689, 690, 691, 693, 694, 695, 696, 698, 699, 700, 701, 703, 704, 705, 707, 708, 709, 710, 712, 713, 714, 716, 717, 718, 720, 721, 722, 724, 725, 726, 727, 728, 729, 730, 731, 732, 733, 734, 735, 736, 744, 745, 747, 748, 750, 751, 753, 754, 755, 756, and variants thereof having at least 70%, 75%, 80%, 85%, 90%, 95%, 98%, and 99% sequence identity thereto.

Modified defensin peptides and modified defensin C-terminal fragments include peptides comprising, consisting essentially of, or consisting of the sequences wherein a gamma-core consensus peptide of a defensin or defensin C-terminal fragment is replaced by a modified gamma-core peptide. In certain embodiments, such replacement of the gamma-core consensus peptide (e.g., GXCX3-9C or GXCX3-22C) by a modified gamma-core peptide can be accomplished by substituting one or more amino acids in the gamma-core consensus peptide with one or more amino acids set forth in a modified gamma-core peptide. In certain embodiments, the substitutions can comprise substitution of the C-terminal cysteine residue in the wild-type gamma-core consensus peptide of a defensin peptide (e.g., the cysteine corresponding to C6 in FIGS. 1 and 2) or of a defensin C-terminal peptide fragment with: (i) a phenylalanine, tryptophan, or tyrosine residue; or (ii) a phenylalanine, tryptophan, tyrosine, leucine, valine, isoleucine, or methionine residue). In certain embodiments, the substitutions can comprise substitution of both the C-terminal cysteine residue in the wild-type gamma-core consensus peptide (e.g., the cysteine corresponding to C6 in FIGS. 1 and 2) and the closest C-terminal cysteine in a defensin peptide or defensin C-terminal peptide fragment (e.g., the cysteine corresponding to C7 in FIGS. 1 and 2) with: (i) a phenylalanine, tryptophan, or tyrosine residue; or (ii) a phenylalanine, tryptophan, tyrosine, leucine, valine, isoleucine, or methionine residue). In certain embodiments, the substitutions can comprise substitution of the C-terminal cysteine residue in the wild-type gamma-core consensus peptide (e.g., the cysteine corresponding to C6 in FIGS. 1 and 2) and the closest C-terminal cysteine in a defensin peptide or defensin C-terminal peptide fragment (e.g., the cysteine corresponding to C7 in FIGS. 1 and 2) with: (i) a phenylalanine, tryptophan, or tyrosine residue; or (ii) a phenylalanine, tryptophan, tyrosine, leucine, valine, isoleucine, or methionine residue and an additional substitution of the amino acid located between the two aforementioned cysteines (e.g., the amino acid residue between the cysteines corresponding to the C6 and C7 defensins in FIGS. 1 and 2) with: (i) a phenylalanine, tryptophan, or tyrosine residue; (ii) a phenylalanine, tryptophan, tyrosine, leucine, valine, isoleucine, or methionine residue; or (iii) a lysine, arginine, or histidine residue. In certain embodiments, one or more additional amino acids located between the N-terminal and C-terminal cysteine of the wild-type gamma-core peptide are substituted (e.g., substitutions of residues corresponding to X3-22 in a GXCX3-22C gamma-core peptide). Substitutions of residues corresponding to X3-22 in a GXCX3-22C gamma-core peptide can include amino acid substitutions which increase or maintain the net positive charge of the peptide at neutral pH and/or increase or maintain the hydrophobicity of the peptide. In certain embodiments, substitutions of residues corresponding to X3-22 in a GXCX3-22C gamma-core peptide can thus include substitutions of one or more anionic aspartate or glutamate residues with cationic amino acids such as arginine, histidine, and lysine and/or with neutral nonpolar (hydrophobic) amino acids such as alanine, leucine, isoleucine, valine, proline, phenylalanine, tryptophan, and methionine. Wild-type defensins comprising wild-type gamma-core peptides that can be replaced or substituted as described above and elsewhere herein to obtain anti-microbial peptides include SEQ ID NO: 11-41, 81 to 533, 534, 538, 542, 546, 551, 555, 560, 565, 569, 573, 578, 583, 588, 593, 598, 603, 608, 612, 616, 620, 624, 628, 633, 637, 641, 645, 649, 653, 657, 662, 666, 670, 674, 679, 683, 688, 692, 697, 702, 706, 711, 715, 719, 723, 743, 746, 749, or 752 and gamma-core peptide containing variants thereof having at least 70%, 75%, 80%, 85%, 90%, 95%, 98%, and 99% identity thereto. Modified C-terminal fragments comprising modified gamma-core peptides and deletions of wild-type defensins that include SEQ ID NO: 11-41, 81 to 533, 534, 538, 542, 546, 551, 555, 560, 565, 569, 573, 578, 583, 588, 593, 598, 603, 608, 612, 616, 620, 624, 628, 633, 637, 641, 645, 649, 653, 657, 662, 666, 670, 674, 679, 683, 688, 692, 697, 702, 706, 711, 715, 719, 723, 743, 746, 749, or 752 and gamma-core peptide containing variants thereof having at least 70%, 75%, 80%, 85%, 90%, 95%, 98%, and 99% identity thereto are also provided herein. Modified defensin peptides and modified C-terminal variants thereof also include those set forth below in Table 1. In Table 1, wild-type defensins and the modified defensin peptides or the modified C-terminal fragments comprising modified gamma-core peptides with amino acid substitutions in the wild-type defensin peptide are set forth sequentially. For example, SEQ ID NO: 535 and 536 comprise modifications of the wild-type SEQ ID NO: 534 defensin while SEQ ID NO: 537 comprises a modified C-terminal fragment of SEQ ID NO: 534. In certain embodiments, the modified defensin peptide disclosed above has at least 70%, 75%, 80%, 85%, 90%, 95%, 98%, 99%, or 99.5% sequence identity to SEQ ID NO: 536, 540, 544, 549, 553, 558, 563, 567, 571, 576, 581, 586, 591, 596, 601, 606, 610, 614, 618, 622, 626, 631, 635, 639, 643, 647, 651, 655, 660, 664, 668, 672, 677, 681, 686, 690, 695, 700, 704, 709, 717, 721, 726, 745, 748, 751, 754, or 756.

TABLE 1
Partial listing of exemplary wild-type, modified defensin
peptide, and modified C-terminal fragments

| SEQ ID NO: | Source | Comments |
|---|---|---|
| 534 | Pinus sylvestris | wild-type defensin |
| 535 | synthetic | modified defensin |
| 536 | synthetic | modified defensin |
| 537 | synthetic | modified C-terminal |
| | | fragment |
| 538 | Pinus sylvestris | wild-type defensin |
| 539 | synthetic | modified defensin |
| 540 | synthetic | modified defensin |
| 541 | synthetic | modified C-terminal |
| | | fragment |
| 542 | Eucalyptus grandis | wild-type defensin |
| 543 | synthetic | modified defensin |
| 544 | synthetic | modified defensin |
| 545 | synthetic | modified C-terminal |
| | | fragment |
| 546 | Striga asiatica | wild-type defensin |
| 547 | synthetic | modified defensin |
| 548 | synthetic | modified defensin |
| 549 | synthetic | modified defensin |
| 550 | synthetic | modified C-terminal |
| | | fragment |
| 551 | Panax ginseng | wild-type defensin |
| 552 | synthetic | modified defensin |
| 553 | synthetic | modified defensin |
| 554 | synthetic | modified C-terminal |
| | | fragment |
| 555 | Erythranthe guttata | wild-type defensin |
| 556 | synthetic | modified defensin |
| 557 | synthetic | modified defensin |
| 558 | synthetic | modified defensin |
| 559 | synthetic | modified C-terminal |
| | | fragment |
| 560 | Raphanus sativus | wild-type defensin |
| 561 | synthetic | modified defensin |
| 562 | synthetic | modified defensin |
| 563 | synthetic | modified defensin |
| 564 | synthetic | modified C-terminal |
| | | fragment |
| 565 | Fraxinus excelsior | wild-type defensin |
| 566 | synthetic | modified defensin |
| 567 | synthetic | modified defensin |
| 568 | synthetic | modified C-terminal |
| | | fragment |
| 569 | Crambe maritima | wild-type defensin |
| 570 | synthetic | modified defensin |
| 571 | synthetic | modified defensin |
| 572 | synthetic | modified C-terminal |
| | | fragment |
| 573 | Chlorophytum | wild-type defensin |
| | laxum | |
| 574 | synthetic | modified defensin |
| 575 | synthetic | modified defensin |
| 576 | synthetic | modified defensin |
| 577 | synthetic | modified C-terminal |
| | | fragment |
| 578 | Setaria viridis | wild-type defensin |
| 579 | synthetic | modified defensin |
| 580 | synthetic | modified defensin |
| 581 | synthetic | modified defensin |
| 582 | synthetic | modified C-terminal |
| | | fragment |
| 583 | Trifolium pratense | wild-type defensin |
| 584 | synthetic | modified defensin |
| 585 | synthetic | modified defensin |
| 586 | synthetic | modified defensin |
| 587 | synthetic | modified C-terminal |
| | | fragment |
| 588 | Fragaria vesca | wild-type defensin |
| 589 | synthetic | modified defensin |
| 590 | synthetic | modified defensin |
| 591 | synthetic | modified defensin |
| 592 | synthetic | modified C-terminal |
| | | fragment |
| 593 | Solanum | wild-type defensin |
| | lycopersicum | |
| 594 | synthetic | modified defensin |
| 595 | synthetic | modified defensin |
| 596 | synthetic | modified defensin |
| 597 | synthetic | modified C-terminal |
| | | fragment |
| 598 | Mimulus guttatus | wild-type defensin |
| 599 | synthetic | modified defensin |
| 600 | synthetic | modified defensin |
| 601 | synthetic | modified defensin |
| 602 | synthetic | modified C-terminal |
| | | fragment |
| 603 | Thlaspi arvense | wild-type defensin |
| 604 | synthetic | modified defensin |
| 605 | synthetic | modified defensin |
| 606 | synthetic | modified defensin |
| 607 | synthetic | modified C-terminal |
| | | fragment |
| 608 | Rubus chingii | wild-type defensin |
| 609 | synthetic | modified defensin |
| 610 | synthetic | modified defensin |
| 611 | synthetic | modified C-terminal |
| | | fragment |
| 612 | Prunus dulcis | wild-type defensin |
| 613 | synthetic | modified defensin |
| 614 | synthetic | modified defensin |
| 615 | synthetic | modified C-terminal |
| | | fragment |
| 616 | Theobroma cacao | wild-type defensin |
| 617 | synthetic | modified defensin |
| 618 | synthetic | modified defensin |
| 619 | synthetic | modified C-terminal |
| | | fragment |
| 620 | Prunus armeniaca | wild-type defensin |
| 621 | synthetic | modified defensin |
| 622 | synthetic | modified defensin |
| 623 | synthetic | modified C-terminal |
| | | fragment |
| 624 | Nicotiana sylvestris | wild-type defensin |
| 625 | synthetic | modified defensin |
| 626 | synthetic | modified defensin |
| 627 | synthetic | modified C-terminal |
| | | fragment |
| 628 | Cajanus cajan | wild-type defensin |
| 629 | synthetic | modified defensin |
| 630 | synthetic | modified defensin |
| 631 | synthetic | modified defensin |
| 632 | synthetic | modified C-terminal |
| | | fragment |
| 633 | Eucalyptus grandis | wild-type defensin |
| 634 | synthetic | modified defensin |
| 635 | synthetic | modified defensin |
| 636 | synthetic | modified C-terminal |
| | | fragment |
| 637 | Eucalyptus grandis | wild-type defensin |
| 638 | synthetic | modified defensin |
| 639 | synthetic | modified defensin |
| 640 | synthetic | modified C-terminal |
| | | fragment |
| 641 | Eucalyptus grandis | wild-type defensin |
| 642 | synthetic | modified defensin |
| 643 | synthetic | modified defensin |
| 644 | synthetic | modified C-terminal |
| | | fragment |
| 645 | Eucalyptus grandis | wild-type defensin |
| 646 | synthetic | modified defensin |
| 647 | synthetic | modified defensin |
| 648 | synthetic | modified C-terminal |
| | | fragment |
| 649 | Fragaria x anannasa | wild-type defensin |
| 650 | synthetic | modified defensin |
| 651 | synthetic | modified defensin |
| 652 | synthetic | modified C-terminal |
| | | fragment |
| 653 | Fragaria x anannasa | wild-type defensin |
| 654 | synthetic | modified defensin |
| 655 | synthetic | modified defensin |
| 656 | synthetic | modified C-terminal |
| | | fragment |
| 657 | Fragaria x anannasa | wild-type defensin |
| 658 | synthetic | modified defensin |
| 659 | synthetic | modified defensin |
| 660 | synthetic | modified defensin |
| 661 | synthetic | modified C-terminal |
| | | fragment |
| 662 | Fragaria vesca | wild-type defensin |
| 663 | synthetic | modified defensin |
| 664 | synthetic | modified defensin |
| 665 | synthetic | modified C-terminal |
| | | fragment |
| 666 | Glycine max | wild-type defensin |
| 667 | synthetic | modified defensin |
| 668 | synthetic | modified defensin |
| 669 | synthetic | modified C-terminal |
| | | fragment |
| 670 | Gossypium | wild-type defensin |
| | hirsutum | |
| 671 | synthetic | modified defensin |
| 672 | synthetic | modified defensin |
| 673 | synthetic | modified C-terminal |
| | | fragment |
| 674 | Gossypium | wild-type defensin |
| | hirsutum | |
| 675 | synthetic | modified defensin |
| 676 | synthetic | modified defensin |
| 677 | synthetic | modified defensin |
| 678 | synthetic | modified C-terminal |
| | | fragment |
| 679 | Lupinus albus | wild-type defensin |
| 680 | synthetic | modified defensin |
| 681 | synthetic | modified defensin |
| 682 | synthetic | modified C-terminal |
| | | fragment |
| 683 | Lupinus albus | wild-type defensin |
| 684 | synthetic | modified defensin |
| 685 | synthetic | modified defensin |
| 686 | synthetic | modified defensin |
| 687 | synthetic | modified C-terminal |
| | | fragment |
| 688 | Manihot esculenta | wild-type defensin |
| 689 | synthetic | modified defensin |
| 690 | synthetic | modified defensin |
| 691 | synthetic | modified C-terminal |
| | | fragment |
| 692 | Manihot esculenta | wild-type defensin |
| 693 | synthetic | modified defensin |
| 694 | synthetic | modified defensin |
| 695 | synthetic | modified defensin |
| 696 | synthetic | modified C-terminal |
| | | fragment |
| 697 | Malus domestica | wild-type defensin |
| 698 | synthetic | modified defensin |
| 699 | synthetic | modified defensin |
| 700 | synthetic | modified defensin |
| 701 | synthetic | modified C-terminal |
| | | fragment |
| 702 | Setaria italica | wild-type defensin |
| 703 | synthetic | modified defensin |
| 704 | synthetic | modified defensin |
| 705 | synthetic | modified C-terminal |
| | | fragment |
| 706 | Setaria italica | wild-type defensin |
| 707 | synthetic | modified defensin |
| 708 | synthetic | modified defensin |
| 709 | synthetic | modified defensin |
| 710 | synthetic | modified C-terminal |
| | | fragment |
| 711 | Setaria italica | wild-type defensin |
| 712 | synthetic | modified defensin |
| 713 | synthetic | modified defensin |
| 714 | synthetic | modified C-terminal |
| | | fragment |
| 715 | Setaria italica | wild-type defensin |
| 716 | synthetic | modified defensin |
| 717 | synthetic | modified defensin |
| 718 | synthetic | modified C-terminal |
| | | fragment |
| 719 | Striga asiatica | wild-type defensin |
| 720 | synthetic | modified defensin |
| 721 | synthetic | modified defensin |
| 722 | synthetic | modified C-terminal |
| | | fragment |
| 723 | Rubus argutus | wild-type defensin |
| 724 | synthetic | modified defensin |
| 725 | synthetic | modified defensin |
| 726 | synthetic | modified defensin |
| 727 | synthetic | modified defensin C- |
| | | terminal fragment |
| 743 | Olea europaea | wild-type defensin |
| 744 | synthetic | modified defensin |
| 745 | synthetic | modified defensin |
| 746 | Medicago truncatula | wild-type defensin |
| 747 | synthetic | modified defensin |
| 748 | synthetic | modified defensin |
| 749 | Medicago truncatula | wild-type defensin |
| 750 | synthetic | modified defensin |
| 751 | synthetic | modified defensin |
| 752 | Sorghum bicolor | wild-type defensin |
| 753 | synthetic | modified defensin |
| 754 | synthetic | modified defensin |
| 755 | synthetic | modified defensin |
| 756 | synthetic | modified defensin |
| 828 | synthetic | Modified defensin C- |
| | | terminal fragment |
| 829 | synthetic | Modified defensin C- |
| | | terminal fragment |
| 830 | synthetic | Modified defensin C- |
| | | terminal fragment |
| 831 | synthetic | Modified defensin C- |
| | | terminal fragment |
| 832 | synthetic | Modified defensin C- |
| | | terminal fragment |
| 833 | synthetic | Modified defensin C- |
| | | terminal fragment |

In certain embodiments, modified defensin or defensin-like peptides of this disclosure are characterized as containing a modified defensin gamma-core peptide that is involved in the antifungal activity of plant defensins. A gamma-core peptide or a modified gamma-core peptide typically contains a net positive charge and has at least one hydrophobic amino acid. In certain embodiments, a modified defensin or defensin-like peptide can comprise the gamma-core consensus sequence of GXCX3-9C or GXCX3-22C wherein X is any amino acid other than cysteine and wherein the 3 to 9 or 3 to 22 amino acid residues other than cysteine are located between the two cysteine residues of the gamma-core consensus peptide. In certain embodiments, a modified defensin or modified defensin-like peptide comprises a modified gamma-core peptide comprising the peptide sequence GXCX3-9(F/W/Y), GXCX3-9(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), wherein 3-9, 9-22, or 16-22 amino acid residues other than cysteine are followed by a C-terminal phenylalanine, tryptophan, tyrosine, leucine, valine, isoleucine, or methionine residue. In certain embodiments, a modified defensin or modified defensin-like peptide comprises a modified gamma-core peptide comprising the peptide sequence GXCX3-9(F/W/Y)(F/W/Y/M)(F/W/Y), GXCX3-9(F/W/Y/L/V/I/M), (F/W/Y/L/V/I/M)(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), or GXCX16-22 (F/W/Y/L/V/I/M)(F/W/Y/L/V/I/M)(F/W/Y/L/V/I/M), wherein 3-9, 9-22, or 16-22 amino acid residues other than cysteine are followed at their C-terminus by: (i) a phenylalanine, tryptophan, tyrosine, leucine, valine, isoleucine, or methionine residue, where (i) is followed at its C-terminus by (ii) a phenylalanine, tryptophan, tyrosine, leucine, valine, isoleucine, or methionine residue, and where (ii) is followed at its C-terminus by (iii) a phenylalanine, tryptophan, tyrosine, leucine, valine, isoleucine, or methionine residue. In certain embodiments, a modified defensin or modified defensin-like peptide comprises a modified gamma-core peptide comprising the peptide sequence GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), wherein 3-9, 9-22, or 16-22 amino acid residues other than cysteine are followed at their C-terminus by: (i) a phenylalanine, tryptophan, tyrosine, leucine, valine, isoleucine, or methionine residue, where (i) is followed at its C-terminus by (ii) an arginine, lysine, or histidine residue, and where (ii) is followed at its C-terminus by (iii) a phenylalanine, tryptophan, tyrosine, leucine, valine, isoleucine, or methionine residue. In certain embodiments, a modified defensin or defensin-like peptide comprises any of the aforementioned modified gamma core sequences wherein X is preferentially selected from cationic and/or hydrophobic amino acids, wherein any of the variable (X) amino acid residues of the modified gamma-core comprise amino acid residues that maintain or increase the number of cationic and/or hydrophobic amino acids found in the wild-type defensin wild-type defensin comprising the polypeptide of SEQ ID NO: 11-41, 81 to 533, 534, 538, 542, 546, 551, 555, 560, 565, 569, 573, 578, 583, 588, 593, 598, 603, 608, 612, 616, 620, 624, 628, 633, 637, 641, 645, 649, 653, 657, 662, 666, 670, 674, 679, 683, 688, 692, 697, 702, 706, 711, 715, 719, 723, 743, 746, 749, or 752 or found in the wild-type defensin-like peptide comprising the polypeptide of SEQ ID NO: 728, SEQ ID NO: 730, SEQ ID NO: 732, or SEQ ID NO: 734. It is believed that the gamma-core peptide is involved in phospholipid- and/or sphingolipid-binding while specific amino acids outside the gamma-core motif are also involved in phospholipid- and sphingolipid-binding. In certain embodiments, the X3-22 amino acid sequence between the cysteine corresponding to C6 and the cysteine corresponding to C7 in the corresponding region in a modified defensin or defensin-like peptide) also contributes to antimicrobial activity. In certain embodiments, modified defensin peptides and defensin like peptides having one or more of any of the aforementioned modified gamma-core sequences include the peptides of SEQ ID NO: 535, 536, 537, 539, 540, 541, 543, 544, 545, 547, 548, 549, 550, 552, 553, 554, 556, 557, 558, 559, 561, 562, 563, 564, 566, 567, 568, 570, 571, 572, 574, 575, 576, 577, 579, 580, 581, 582, 584, 585, 586, 587, 589, 590, 591, 592, 594, 595, 596, 597, 599, 600, 601, 602, 604, 605, 606, 607, 609, 610, 611, 613, 614, 615, 617, 618, 619, 621, 622, 623, 625, 626, 627, 629, 630, 631, 632, 634, 635, 636, 638, 639, 640, 642, 643, 644, 646, 647, 648, 650, 651, 652, 654, 655, 656, 658, 659, 660, 661, 663, 664, 665, 667, 668, 669, 671, 672, 673, 675, 676, 677, 678, 680, 681, 682, 684, 685, 686, 687, 689, 690, 691, 693, 694, 695, 696, 698, 699, 700, 701, 703, 704, 705, 707, 708, 709, 710, 712, 713, 714, 716, 717, 718, 720, 721, 722, 724, 725, 726, 727, 728, 729, 730, 731, 732, 733, 734, 735, 736, 744, 745, 747, 748, 750, 751, 753, 754, 755, 756, and variants thereof having at least 70%, 75%, 80%, 85%, 90%, 95%, 98%, and 99% sequence identity thereto.

Modified defensin or defensin-like peptides comprising modified C-terminal fragments thereof are also provided herein. In certain embodiments, the modified defensin or defensin-like C-terminal fragments comprise, consist essentially of, or consist of: (i) 30 amino acid residues or less; or (ii) 15, 16, or 17 to 30 amino acid residues. In certain embodiments, the C-terminal fragment has a net positive charge of at least 3 and a hydrophobic amino acid content at least 18%. In certain embodiments, modified defensin or defensin-like proteins comprising C-terminal fragments contain a total of two cysteine peptide, and optionally comprises a disulfide bond between the two cysteine residues in the modified C-terminal fragment. In certain embodiments, the aforementioned modified defensin peptide C-terminal fragments comprise, consist essentially of, or consist of a peptide corresponding to the C-terminal end of a defensin peptide comprising the C5 and C8 conserved cysteines wherein a tryptophan, tyrosine or phenylalanine substitution of the residues corresponding to the C6 through C7 residues results in a defensin C-terminal fragment comprising a modified gamma-core variant sequence GXCX3-9(F/W/Y)(F/W/Y)(F/W/Y), GXCX3-22(F/W/Y)(F/W/Y)(F/W/Y), GXCX16-22 (F/W/Y)(F/W/Y)(F/W/Y), GXCX3-9(F/W/Y), GXCX16-22(F/W/Y), or GXCX3-22(F/W/Y). In certain embodiments, the aforementioned modified defensin peptide C-terminal fragments comprise, consist essentially of, or consist of a peptide corresponding to the C-terminal end of a defensin peptide comprising the C5 and C8 conserved cysteines wherein a tryptophan, tyrosine, phenylalanine, leucine, valine, isoleucine, or methionine substitution of the residues corresponding to the C6 through C7 residues results in a defensin C-terminal fragment comprising a modified gamma-core variant sequence GXCX3-9(F/W/Y/L/V/I/M)(F/W/Y/L/V/I/M)(F/W/Y/L/V/I/M), GXCX16-22(F/W/Y/L/V/I/M)(F/W/Y/L/V/I/M)(F/W/Y/L/V/I/M), or GXCX3-22 (F/W/Y/L/V/I/M)(F/W/Y/L/V/I/M)(F/W/Y/L/V/I/M). In certain embodiments, the modified defensin of defensin like C-terminal fragment will comprise a modified gamma-core sequence comprising GXCX3-9(F/W/Y)(R/K/H)(F/W/Y), GXCX3-9(F/W/Y/L/V/I/M) GXCX9-22(F/W/Y)(R/K/H)(F/W/Y), GXCX16-(R/K/H)(F/W/Y/L/V/I/M), 22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M). In certain embodiments, the defensin or defensin-like C-terminal fragment comprises any of the aforementioned modified gamma-core consensus sequences, wherein X is not cysteine and is preferentially selected from cationic (e.g. R, K, or H) and/or hydrophobic amino acids (e.g., F/W/Y/L/V/I/M). In certain embodiments, the aforementioned defensin or defensin-like protein C-terminal fragment can comprise a peptide having just two (2) cysteine residues, where the two (2) cysteine residues optionally correspond to the conserved C5 and C8 cysteines of a reference or wild-type defensin peptide (e.g., as shown in FIG. 2). In certain embodiments, the aforementioned C-terminal fragments can comprise N-terminal deletions of about 1-35 amino acids and amino acid substitutions in at least the gamma-core peptide of a wild-type defensin polypeptide of SEQ ID NO: 11-41, 81 to 533, 534, 538, 542, 546, 551, 555, 560, 565, 569, 573, 578, 583, 588, 593, 598, 603, 608, 612, 616, 620, 624, 628, 633, 637, 641, 645, 649, 653, 657, 662, 666, 670, 674, 679, 683, 688, 692, 697, 702, 706, 711, 715, 719, 723, 743, 746, 749, 752, or a variant thereof having at least 70%, 75%, 80%, 90%, or 95% sequence identity thereto. In certain embodiments, the aforementioned C-terminal fragments can comprise N-terminal deletions of about 1-35 amino acids and amino acid substitutions in at least the gamma-core peptide of a wild-type defensin-like polypeptide of SEQ ID NO: 728, SEQ ID NO: 730, SEQ ID NO: 732, SEQ ID NO: 734 or a variant thereof having at least 70%, 75%, 80%, 90%, or 95% sequence identity thereto. Exemplary modified defensin C-terminal fragments include those comprising, consisting essentially of those set forth in Table 1 and those comprising, consisting essentially of, or consisting of peptides corresponding to amino acid residues 32-47 of SEQ ID NO: 742, amino acid residues 32-53 of SEQ ID NO: 745, amino acid residues 31-45 of SEQ ID NO: 748, amino acid residues 33-50 of SEQ ID NO: 751, and amino acid residues 34-49 of SEQ ID NO: 754 (e.g., as depicted in FIG. 2). Exemplary modified defensin C-terminal fragments can also comprise, consist essentially of, or consist of the peptides of SEQ ID NO: 828, 829, 830, 831, 832, or 833. Variants of any of the aforementioned C-terminal fragments comprising a conservative amino acid substitution of 1 to 2, 3, 4, or 5 amino acid residues are also provided.

In certain embodiments, defensin-like peptides and defensin-like C-terminal fragments, which are optionally isolated are provided. In certain embodiments, such defensin-like peptides comprise the peptides of SEQ ID NO: 728, 730, 732, 734 or variants thereof having at least 70%, 75%, 80%, 85%, 90%, 95%, 98%, or 99% sequence identity thereto. In certain embodiments, defensin-like C-terminal fragments comprising a deletion of 1 to about 35 N-terminal amino acid residues of SEQ ID NO: 729, 731, 733, 735, 736 or variants thereof having at least 70%, 75%, 80%, 85%, 90%, 95%, 98%, or 99% sequence identity thereto are provided.

In certain embodiments, modified defensin, modified defensin-like peptides, defensin-like peptides, and C-terminal fragments thereof provided herein will have a net positive charge of at least about 5.8 at neutral pH and/or a hydrophobicity percentage of at least about 15%. In certain embodiments, a first structural feature of the modified defensin or defensin-like peptides is a net positive charge at neutral pH. In certain embodiments, the modified defensin or defensin-like peptides will have a net positive charge at neutral pH of at least +2, +3, +3.5, +4, +5, +6, +7, +8, +9, or +10. In certain embodiments, the modified defensin or defensin-like peptides will have a net positive charge at neutral pH of at least +3, +3.5, +4, +5, +6, or +7 to about +8, +9, or +10. In certain embodiments, the hydrophobicity percentage of such modified defensin or defensin-like peptides is at least about 15% to 30%, about 16% to 19%, or about 28% to 30%. In certain embodiments, the aforementioned modified defensin or defensin-like peptides comprise, consist essentially of, or consist of: (i) 30 amino acid residues or less; or (ii) 15, 16, or 17 to 30 amino acid residues. In certain embodiments, any of the aforementioned modified defensin or defensin-like peptides comprise a peptide having just two (2) cysteine residues and optionally comprise the two cysteine residues corresponding to the conserved C5 and C8 cysteines of a reference defensin C-terminal peptide. In certain embodiments, modified defensin peptides and defensin like peptides having one or more of any of the aforementioned net positive charges and/or hydrophobicity percentages include the peptides of SEQ ID NO: 535, 536, 537, 539, 540, 541, 543, 544, 545, 547, 548, 549, 550, 552, 553, 554, 556, 557, 558, 559, 561, 562, 563, 564, 566, 567, 568, 570, 571, 572, 574, 575, 576, 577, 579, 580, 581, 582, 584, 585, 586, 587, 589, 590, 591, 592, 594, 595, 596, 597, 599, 600, 601, 602, 604, 605, 606, 607, 609, 610, 611, 613, 614, 615, 617, 618, 619, 621, 622, 623, 625, 626, 627, 629, 630, 631, 632, 634, 635, 636, 638, 639, 640, 642, 643, 644, 646, 647, 648, 650, 651, 652, 654, 655, 656, 658, 659, 660, 661, 663, 664, 665, 667, 668, 669, 671, 672, 673, 675, 676, 677, 678, 680, 681, 682, 684, 685, 686, 687, 689, 690, 691, 693, 694, 695, 696, 698, 699, 700, 701, 703, 704, 705, 707, 708, 709, 710, 712, 713, 714, 716, 717, 718, 720, 721, 722, 724, 725, 726, 727, 728, 729, 730, 731, 732, 733, 734, 735, 736, 744, 745, 747, 748, 750, 751, 753, 754, 755, 756, and variants thereof having at least 70%, 75%, 80%, 85%, 90%, 95%, 98%, and 99% sequence identity thereto.

In certain embodiments, modified defensin, modified defensin-like, or defensin-like peptides provided herein (e.g., in Table 1, FIG. 2, and the sequence listing) and variants thereof can comprise amino acid substitutions which increase or maintain the net positive charge of the peptide at neutral pH and/or increase or maintain the hydrophobicity of the peptide. Amino acid substitutions in which can maintain net positive charge of the modified defensin, modified defensin-like, or defensin-like peptide at neutral pH include substitution of a lysine, arginine, or histidine residue in the modified defensin, modified defensin-like, or defensin-like peptide with a different amino acid residue selected from the group consisting of lysine, arginine, Dab (diaminobutyric acid), or other non-naturally occurring amino acid which is positively charged at neutral pH. Amino acid substitutions in the modified defensin, modified defensin-like, or defensin-like peptides which can increase net positive charge at neutral pH include substitution of a polar (e.g. cysteine or threonine) or non-polar (e.g., glycine) residue in modified defensin, modified defensin-like, or defensin-like peptides with a different amino acid residue selected from the group consisting of lysine, arginine, Dab (diaminobutyric acid), or other non-naturally occurring amino acid residue which is positively charged at neutral pH. Amino acid substitutions in the modified defensin, modified defensin-like, or defensin-like peptides which can maintain hydrophobicity of the peptide include substitution of a glycine, valine, phenylalanine, or isoleucine residue in the modified defensin, modified defensin-like, or defensin-like peptides with a different amino acid residue selected from the group consisting of glycine, alanine, valine, leucine, phenylalanine, isoleucine, or methionine. Amino acid substitutions in modified defensin, modified defensin-like, or defensin-like peptides which can increase hydrophobicity of the peptide include substitution of a polar (e.g. cysteine or threonine) residue in the modified defensin, modified defensin-like, or defensin-like peptides with a different amino acid residue selected from the group consisting of glycine, alanine, valine, leucine, phenylalanine, or isoleucine. In certain embodiments, such substitutions which increase or maintain net positive charge or hydrophobicity of the peptide will comprise a modified defensin or defensin-like peptide having the gamma-core consensus sequence of GXCX3-9C or GXCX9-22C. In certain embodiments, such substitutions which increase or maintain net positive charge or hydrophobicity of the peptide will comprise a modified defensin or defensin-like peptide having a modified gamma-core variant sequence provided herein. In certain embodiments, such substitutions which increase or maintain net positive charge or hydrophobicity of the peptide will comprise a modified defensin or defensin-like peptide having the modified gamma-core variant sequence can comprise a modified gamma-core variant sequence of GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M)(F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M). In certain embodiments, the aforementioned modified defensin or defensin-like peptides comprise, consist essentially of, or consist of: (i) 30 amino acid residues or less; or (ii) 15, 16, or 17 to 22, 24, 16, 28 or 30 amino acid residues. In certain embodiments, any of the aforementioned modified defensin or defensin-like peptides comprise a peptide having just two (2) cysteine residues and optionally comprise the two cysteine residues corresponding to the conserved C5 and C8 cysteines of a reference defensin C-terminal peptide. In certain embodiments, modified defensin peptides and defensin like peptides which can be substituted as noted above or elsewhere herein include the peptides of SEQ ID NO: 535, 536, 537, 539, 540, 541, 543, 544, 545, 547, 548, 549, 550, 552, 553, 554, 556, 557, 558, 559, 561, 562, 563, 564, 566, 567, 568, 570, 571, 572, 574, 575, 576, 577, 579, 580, 581, 582, 584, 585, 586, 587, 589, 590, 591, 592, 594, 595, 596, 597, 599, 600, 601, 602, 604, 605, 606, 607, 609, 610, 611, 613, 614, 615, 617, 618, 619, 621, 622, 623, 625, 626, 627, 629, 630, 631, 632, 634, 635, 636, 638, 639, 640, 642, 643, 644, 646, 647, 648, 650, 651, 652, 654, 655, 656, 658, 659, 660, 661, 663, 664, 665, 667, 668, 669, 671, 672, 673, 675, 676, 677, 678, 680, 681, 682, 684, 685, 686, 687, 689, 690, 691, 693, 694, 695, 696, 698, 699, 700, 701, 703, 704, 705, 707, 708, 709, 710, 712, 713, 714, 716, 717, 718, 720, 721, 722, 724, 725, 726, 727, 728, 729, 730, 731, 732, 733, 734, 735, 736, 744, 745, 747, 748, 750, 751, 753, 754, 755, and 756. In certain embodiments, modified defensin peptides which can be substituted as noted above or elsewhere herein include the peptides of SEQ ID NO: 11-41, 81 to 533, 534, 538, 542, 546, 551, 555, 560, 565, 569, 573, 578, 583, 588, 593, 598, 603, 608, 612, 616, 620, 624, 628, 633, 637, 641, 645, 649, 653, 657, 662, 666, 670, 674, 679, 683, 688, 692, 697, 702, 706, 711, 715, 719, 723, 743, 746, 749, or 752 wherein the wild-type gamma-core consensus peptide GXCX3-9C or GXCX3-22C of the wild-type defensin or wild-type defensin-like peptide is replaced by a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M) (F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M) and optionally wherein N-terminal residues 1-35 are deleted.

In certain embodiments, one or more amino acids in any of the aforementioned or other modified defensin or defensin-like peptide sequences are substituted with another amino acid(s), the charge and polarity of which is similar to that of the original amino acid, i.e., a conservative amino acid substitution. Substitutes for an amino acid within the modified defensin or defensin-like peptide or protein, or defensin peptide sequence can be selected from other members of the class to which the originally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include: (1) acidic (anionic, negatively charged) amino acids such as aspartic acid and glutamic acid; (2) basic (cationic, positively charged) amino acids such as arginine, histidine, and lysine; (3) neutral polar amino acids such as glycine, serine, threonine, cysteine, cystine, tyrosine, asparagine, and glutamine; (4) neutral nonpolar (hydrophobic) amino acids such as alanine, leucine, isoleucine, valine, proline, phenylalanine, tryptophan, and methionine. Conservative amino acid changes within defensin peptide sequences can be made by substituting one amino acid within one of these groups with another amino acid within the same group. Biologically functional equivalents of modified defensin or defensin-like peptides can have 10 or fewer conservative amino acid changes, seven or fewer conservative amino acid changes, or five, four, three, two, or one conservative amino acid changes. The encoding nucleotide sequence (e.g., gene, plasmid DNA, cDNA, or synthetic DNA) will thus have corresponding base substitutions, permitting it to encode biologically functional equivalent forms of the modified defensin or defensin-like peptides. Certain semi-conservative substitutions in modified defensin or defensin-like peptides including: (i) the substitution of a neutral polar amino acid residue with a neutral nonpolar (hydrophobic) amino acid residue; or (ii) the substitution of a neutral nonpolar (hydrophobic) amino acid residue with a neutral polar amino acid residue are also provided. In particular, semi-conservative substitutions of a neutral polar tyrosine residue with a hydrophobic amino acid residue are provided. Biologically functional equivalents of modified defensin or defensin-like peptides can have 10 or fewer semi conservative amino acid changes, seven or fewer semi-conservative amino acid changes, or five, four, three, two, or one semi-conservative amino acid changes. In certain embodiments, modified defensin peptides and defensin like peptides which can be conservatively or semi-conservatively substituted as noted above or elsewhere herein include the peptides of SEQ ID NO: 535, 536, 537, 539, 540, 541, 543, 544, 545, 547, 548, 549, 550, 552, 553, 554, 556, 557, 558, 559, 561, 562, 563, 564, 566, 567, 568, 570, 571, 572, 574, 575, 576, 577, 579, 580, 581, 582, 584, 585, 586, 587, 589, 590, 591, 592, 594, 595, 596, 597, 599, 600, 601, 602, 604, 605, 606, 607, 609, 610, 611, 613, 614, 615, 617, 618, 619, 621, 622, 623, 625, 626, 627, 629, 630, 631, 632, 634, 635, 636, 638, 639, 640, 642, 643, 644, 646, 647, 648, 650, 651, 652, 654, 655, 656, 658, 659, 660, 661, 663, 664, 665, 667, 668, 669, 671, 672, 673, 675, 676, 677, 678, 680, 681, 682, 684, 685, 686, 687, 689, 690, 691, 693, 694, 695, 696, 698, 699, 700, 701, 703, 704, 705, 707, 708, 709, 710, 712, 713, 714, 716, 717, 718, 720, 721, 722, 724, 725, 726, 727, 728, 729, 730, 731, 732, 733, 734, 735, 736, 744, 745, 747, 748, 750, 751, 753, 754, 755, and 756. In certain embodiments, modified defensin peptides which can be conservatively or semi-conservatively substituted as noted above or elsewhere herein include the peptides of SEQ ID NO: 11-41, 81 to 533, 534, 538, 542, 546, 551, 555, 560, 565, 569, 573, 578, 583, 588, 593, 598, 603, 608, 612, 616, 620, 624, 628, 633, 637, 641, 645, 649, 653, 657, 662, 666, 670, 674, 679, 683, 688, 692, 697, 702, 706, 711, 715, 719, 723, 743, 746, 749, or 752 wherein the wild-type gamma-core consensus peptide GXCX3-9C or GXCX3-22C of the wild-type defensin or wild-type defensin-like peptide is replaced by a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M) (F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M) and optionally wherein N-terminal residues 1-35 are deleted.

Nucleic acid molecules encoding any of the aforementioned modified defensin or defensin-like peptides are also provided herein. Recombinant DNA molecules comprising the aforementioned nucleic acid molecules are also provided herein and in particular recombinant DNA molecules comprising a heterologous promoter that is operably linked to the aforementioned nucleic acid molecules are also provided herein.

A modified defensin or defensin-like peptide provided herein can be operably linked to another modified defensin or defensin-like peptide, defensin, or antimicrobial peptide via a spacer peptide sequence that is not susceptible to cleavage by an endoproteinase, including a plant endoproteinase. Such peptide linker sequences that join peptides in multimeric or multi-domain proteins have been disclosed (Argos, 1990; George R A, Heringa (2002)). Examples of suitable peptide sequences from multimeric or multi-domain proteins that can be used as spacer domains include immunoglobulin hinge regions from immunoglobulins, a linker between the lipoyl and E3 binding domain in pyruvate dehydrogenase (Turner et ah, 1993), a linker between the central and C-terminal domains in cysteine proteinase (P9; Mottram et ah, 1989), and functional variants thereof. Spacer peptides for use in the defensin variant proteins can also be wholly or partially synthetic peptide sequences. Such synthetic spacer peptides are designed to provide for a flexible linkage between the at least one modified defensin or defensin-like peptide and another peptide (including a modified defensin or defensin-like peptide or defensin peptide) and to be resistant to cleavage by endogenous plant or other endoproteinases. In certain embodiments, the length of the synthetic spacer peptide can be between about 3, 4, 8, 10, 12, or 16 and about 20, 24, 28, 30, 40, or 50 amino acid residues in length. In certain embodiments, the synthetic spacer peptide can comprise a glycine-rich or glycine/serine containing peptide sequence. The composition and design of peptides suitable for flexible linkage of protein domains described in the literature (Chen et al., 2013) can be adapted for use as spacer peptides in the defensin variant proteins provided herein. Spacer peptides useful for joining defensin monomers described in US Patent Appln. Publications US20190194268 and US20190185877, which are each incorporated herein by reference in their entireties, can also be used to join modified defensin or defensin-like peptides disclosed herein to other modified defensin or defensin-like peptides, defensins, antimicrobial peptides, or other peptides.

A modified defensin or defensin-like peptide provided herein can be operably linked to another modified defensin or defensin-like peptide, defensin, or antimicrobial peptide via a linker peptide sequence that is susceptible to cleavage by an endoproteinase, including a plant endoproteinase. In certain embodiments, the resultant defensin variant protein can be expressed in a cell such that the endoproteinase cleaves the defensin variant protein to provide at least one modified defensin or defensin-like peptide and another peptide (including a modified defensin or defensin-like peptide or defensin peptide). Such defensin variant proteins can be provided in a cellular compartment (e.g., cytoplasm, mitochondria, plastid, vacuole, or endoplasmic reticulum) or extracellular space (i.e., to the apoplast) having an endoproteinase that cleaves the linker peptide. Cleavable linker peptides are disclosed in WO2014078900, Vasivarama and Kirti, 2013a, Frangois et al, Vasivarama and Kirti, 2013b, and WO2017127558 can be used in the defensin variant proteins provided herein.

Expression cassettes that provide for expression of the modified defensin or defensin-like peptide in monocotyledonous plants, dicotyledonous plants, or both can be constructed. Such modified defensin or defensin-like peptide expression cassette construction can be effected either in a plant expression vector or in the genome of a plant. Expression cassettes are DNA constructs wherein various promoter, coding (e.g. modified defensin or defensin-like peptide encoding), and polyadenylation sequences are operably linked. In general, expression cassettes typically comprise a promoter that is operably linked to a sequence of interest, which is operably linked to a polyadenylation or terminator region. In certain instances including the expression of recombinant or edited polynucleotides in monocot plants, it can also be useful to include an intron sequence. When an intron sequence is included it is typically placed in the 5′ untranslated leader region of the recombinant or edited polynucleotide. In certain instances, it can also be useful to incorporate specific 5′ untranslated sequences in a recombinant or edited polynucleotide to enhance transcript stability or to promote efficient translation of the transcript. Expression cassettes and vectors for expression of other defensin peptides or proteins in plants, including those disclosed in U.S. Pat. No. 10,253,328, which is incorporated herein by reference in its entirety, can be adapted for expression of the modified defensin or defensin-like peptides in transgenic plants. Any of the modified defensin or defensin-like peptide expression vectors can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. The aforementioned methods of introducing transgenes are described in US Patent Appl. Pub. No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton), each of which are incorporated herein by reference in their entirety.

In certain embodiments, a plant comprising a recombinant or edited polynucleotide encoding a modified defensin or defensin-like peptide can be obtained by using techniques that provide for site specific insertion of heterologous DNA into the genome of a plant (e.g., by CRISPR, TALEN, or Zinc finger nuclease-mediated gene editing). In certain embodiments, a DNA fragment encoding at least a modified defensin or defensin-like peptide is site specifically integrated into the genome to a plant cell, tissue, part, or whole plant to create a sequence within that genome that encodes a modified defensin or defensin-like peptide. Examples of methods for inserting foreign DNA at specific sites in the plant genome with site-specific nucleases such as meganucleases or zinc-finger nucleases are at least disclosed in Voytas, 2013. Examples of methods for inserting foreign DNA into the plant genome with clustered regularly interspaced short palindromic repeats (CRISPR)-associated (Cas)-guide RNA technology and a Cas endonuclease are at least disclosed by Svitashev et al., 2015; Murovec et al., 2017; Kumar and Jain, 2015; and in US Patent Appl. Pub. No. 20150082478, which is specifically incorporated herein by reference in its entirety.

Expression of modified defensin or defensin-like peptides in yeast is also specifically contemplated herein. The construction of expression vectors for production of heterologous proteins in various yeast genera is well established. In general, such expression vectors typically comprise a promoter that is operably linked to a sequence of interest which is operably linked to a polyadenylation or terminator region. Examples of yeast genera that have been used to successfully express heterologous genes include Candida, Kluveromyces, Hansuela, Pichia, Saccharomyces, Schizosaccharomyces, and Yarrowia. A general description of expression vectors and transformation systems for Saccharomyces is found in Kingsman et al (1985). Expression vectors and transformation systems useful for yeasts other than Saccharomyces are described in Reiser et al (1990). Expression cassettes and vectors for expression of other defensin peptides or proteins in yeast, including those disclosed in U.S. Pat. No. 10,253,328, which is incorporated herein by reference in its entirety, can be adapted for expression of the modified defensin or defensin-like peptides in yeast.

Expression of modified defensin or defensin-like peptides in bacteria is also specifically contemplated herein. The construction of expression vectors for production of heterologous proteins in various bacterial systems have been described. In general, such expression vectors typically comprise a promoter that is operably linked to a sequence encoding peptide sequence(s) of interest (e.g., a signal transit peptide region at the n-terminus of a defensin variant peptide) which is operably linked to a procaryotic terminator region. Examples of bacterial genera that have been used to successfully express heterologous genes include Acinetobacter, Alcaligenes, Azotobacter, Bacillus, Escherichia, Lactobacillus, Lactococcus, Streptomyces, and Pseudomonas. E. coli expression systems useful for production of proteins comprising disulfide bonds and that can be adapted for use in expression of the defensin variant peptides provided herein include those described in are described in Kuddus et al. (2017) Biotechnol Prog 233:1520-1528. doi: 10.1002/btpr.Protein Science 2508, Kiedzierska et al. (2008) Protein Expr Purif 60, 82-88, Chang et al. (2015) Amino Acids 47, 579-587, Buchko et al. (2018) Protein Science 27, 1611-1623, Marques et al. (2008) J Appl Microbiol 106, 1640-1648, and Pazgier et al. (2006) Protein Expr Pur 49, 1-8. Systems for expressing proteins which comprise disulfide bonds can be adapted for expression of the Defensin peptides in E. coli include those disclosed in U.S. Patent Publication No. US 2020/0172915, which is incorporated herein by reference in its entirety, and in Berkmen, M. Protein Expr Purif. 2012; 82(1):240-51. doi: 10.1016/j.pep.2011.10.009.

Other bacterial expression systems that are useful for production of proteins comprising disulfide bonds and that can be adapted for use in expression of the defensin variant peptides provided herein include those described in U.S. Pat. No. 10,604,761, which is incorporated herein by reference in its entirety.

Also provided are antimicrobial compositions for agricultural, pharmaceutical, or veterinary use comprising either an antimicrobial plant, or antimicrobial human or veterinary, pathogenic microbe inhibitory amount (“antimicrobial effective amount”) of one or more the present isolated, purified antimicrobial modified defensin or defensin-like peptides, or biologically functional equivalents thereof. Such compositions can comprise one, or any combination of, modified defensin or defensin-like peptides or proteins disclosed herein, and an agriculturally, pharmaceutically, or veterinary-practicably acceptable carrier, diluent, or excipient. As indicated below, other components relevant in agricultural and therapeutic contexts can be included in such compositions as well. The antimicrobial compositions can be used for inhibiting the growth of, or killing, modified defensin or defensin-like peptide-susceptible pathogenic microbes associated with plant, human or animal microbial infections. Such antimicrobial compositions can be formulated for topical administration, and applied topically to either plants, the plant environment (including soil), or humans or animals. Such antimicrobial compositions can be formulated for enteral, parenteral, and/or intravenous administration of the composition, and administered to a subject in need thereof; such subject can be a human, livestock, poultry, fish, or a companion animal. The modified defensin or defensin-like peptides can be formulated alone, in any combination with one another, and either of these can additionally be formulated in combination with other conventional antimicrobial therapeutic compounds such as, by way of non-limiting example, polyene antimicrobials; imidazole, triazole, and thiazole antimicrobials; allylamines; and echinocandins that are routinely used in human and veterinary medicine. Administration of the compositions that comprise modified defensin or defensin-like peptides to a human or animal subject in need thereof can be accomplished via a variety of routes that include topical application, enteral administration, parenteral administration, and/or intravenous administration. The antimicrobial peptides and compositions can be used to control microbial pathogens or contaminants including: (i) a bacterial pathogen of plants or animals, wherein the bacterial pathogen is optionally a member of the group Enterobacteriaceae and optionally wherein the bacterial pathogen is a Salmonella sp., Escherichia sp., or Listeria sp.; (ii) is a Fusarium sp., Alternaria sp., Aphenomyces sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp., or Stenocarpella sp.; (iii) an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, Coccidiomyces or Blastomyces species; (iv) a Candida species and wherein the Candida species is Candida albicans (C. albicans), C. auris, C. glabrata, C parapsilosis, C. tropicalis, or C. krusei; or (v) dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

Agricultural compositions comprising any of the present modified defensin or defensin-like peptide molecules alone, or in any combination, can be formulated as described in, for example, Winnacker-Kuchler (1986) Chemical Technology, Fourth Edition, Volume 7, Hanser Verlag, Munich; van Falkenberg (1972-1973) Pesticide Formulations, Second Edition, Marcel Dekker, N.Y.; and K. Martens (1979) Spray Drying Handbook, Third Edition, G. Goodwin, Ltd., London. Formulation aids, such as carriers, inert materials, surfactants, solvents, and other additives are also well known in the art, and are described, for example, in Watkins, Handbook of Insecticide Dust Diluents and Carriers, Second Edition, Darland Books, Caldwell, N.J., and Winnacker-Kuchler (1986) Chemical Technology, Fourth Edition, Volume 7, Hanser Verlag, Munich. Using these formulations, it is also possible to prepare mixtures of the present modified defensin or defensin-like peptides and proteins with other pesticidally active substances, fertilizers, and/or growth regulators, etc., in the form of finished formulations or tank mixes.

Whether alone or in combination with other active agents, the present antimicrobial modified defensin or defensin-like peptides can be applied to subjects or plants at a concentration in the range of from about 0.1 μg/ml to about 100 mg/ml, or from about 5 μg/ml to about 5 mg/ml, at a pH in the range of from about 3.0 to about 9.0. Such compositions can be buffered using, for example, phosphate buffers between about 1 mM and 1 M, about 10 mM to about 100 mM, or about 15 mM to about 50 mM. In the case of low buffer concentrations, a salt can be added to increase the ionic strength. In certain embodiments, a sodium salt, including NaCl, in the range of from about 1 mM to about 1 M, about 1 mM, 5 mM, or 10 mM to about 20 mM, 50 mM, 100 mM, 150 mM, or 200 mM, or about 10 mM to about 100 mM, can be added or provided in compositions comprising modified defensin or defensin-like peptides and proteins. In certain embodiments, a potassium salt, including KCl, in the range of about 1 mM, 5 mM, or 10 mM to about 20 mM, 50 mM, 100 mM, 150 mM, or 200 mM can be added or provided in compositions comprising modified defensin or defensin-like peptides and proteins. In certain embodiments, a calcium salt, including CaCl₂), in the range of about 0.1 mM, 0.5 mM, or 1 mM to about 2 mM, 5 mM, 10 mM, or 20 mM can be added or provided in compositions comprising modified defensin or defensin-like peptides.

Numerous conventional microbial antibiotics and chemical antimicrobial agents (e.g., fungicides) with which the present modified defensin or defensin-like peptides and proteins can be combined are described in Worthington and Walker (1983) The Pesticide Manual, Seventh Edition, British Crop Protection Council. These include, for example, polyoxins, nikkomycins, carboxy amides, aromatic carbohydrates, carboxines, morpholines, inhibitors of sterol biosynthesis, and organophosphorus compounds. In addition, azoles, triazoles and echinocandins fungicides can also be used. Other active ingredients which can be formulated in combination with the present antimicrobial peptides and proteins include, for example, insecticides, attractants, sterilizing agents, acaricides, nematicides, and herbicides. U.S. Pat. No. 5,421,839, which is incorporated herein by reference in its entirety, contains a comprehensive summary of the many active agents with which substances such as the present antimicrobial modified defensin or defensin-like peptides and proteins can be formulated.

Agriculturally useful antimicrobial compositions encompassed herein also include those in the form of host cells, such as bacterial and microbial cells, capable of producing the modified defensin or defensin-like peptides and proteins, and which can colonize plants, including roots, shoots, leaves, or other parts of plants. The term “plant-colonizing microorganism” is used herein to refer to a microorganism that is capable of colonizing the any part of the plant itself and/or the plant environment, including, and which can express the present defensin variant antimicrobial peptides and proteins in the plant and/or the plant environment. A plant colonizing micro-organism is one that can exist in symbiotic or non-detrimental relationship with a plant in the plant environment. U.S. Pat. No. 5,229,112, which is incorporated herein by reference in its entirety, discloses a variety of plant-colonizing microorganisms that can be engineered to express antimicrobial peptides and proteins, and methods of use thereof, applicable to the defensin variant antimicrobial peptides and proteins disclosed herein. Plant-colonizing microorganisms expressing the presently disclosed defensin variant antimicrobial peptides and proteins useful in inhibiting microbial growth in plants include bacteria selected from the group consisting of Bacillus spp. including Bacillus thuringiensis, Bacillus israelensis, and Bacillus subtilis, Candidatus Liberibacter asiaticus; Pseudomonas spp.; Arthrobacter spp., Azospyrillum spp., Clavibacter spp., Escherichia spp.; Agrobacterium spp., for example A. radiobacter, Rhizobium spp., Erwinia spp. Azotobacter spp., Azospirillum spp., Klebsiella spp., Alcaligenes spp., Rhizobacterium spp., Xanthomonas spp., Ralstonia spp, and Flavobacterium spp. In certain embodiments, the microorganism is a yeast selected from the group consisting of Saccharomyces cerevisiae, Pichia pastoris, and Pichia methanolica. In certain embodiments, the plant colonizing microorganism can be an endophytic bacteria or microbe. When applying the present modified defensin or defensin-like peptide molecules to the rhizosphere, rhizosphere-colonizing bacteria from the genus Pseudomonas are particularly useful, especially the fluorescent pseudomonads, e.g., Pseudomonas fluorescens, which is especially competitive in the plant rhizosphere and in colonizing the surface of the plant roots in large numbers. Examples of suitable phylloplane (leaf) colonizing bacteria are P. putida, P. syringae, and Erwinia species.

### Embodiments

The following numbered embodiments form part of the disclosure:
1. A peptide comprising the amino acid sequence of a modified defensin or modified defensin-like peptide wherein the wild-type gamma-core consensus peptide GXCX3-9C or GXCX3-22C of the wild-type defensin or wild-type defensin-like peptide is replaced by a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M)(F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), and wherein the peptide has a net positive charge of at least 3 and a hydrophobic amino acid content at least 18%.
2. The peptide of embodiment 1, wherein: (i) the wild-type defensin comprises the polypeptide of SEQ ID NO: 11-41, 81 to 533, 534, 538, 542, 546, 551, 555, 560, 565, 569, 573, 578, 583, 588, 593, 598, 603, 608, 612, 616, 620, 624, 628, 633, 637, 641, 645, 649, 653, 657, 662, 666, 670, 674, 679, 683, 688, 692, 697, 702, 706, 711, 715, 719, 723, 743, 746, 749, 752, or a variant thereof having at least 70%, 75%, 80%, 85%, 90%, 95%, 98%, 99%, or 99.5% sequence identity thereto; or (ii) wherein the wild-type defensin-like peptide comprises the polypeptide of SEQ ID NO: 728, SEQ ID NO: 730, SEQ ID NO: 732, SEQ ID NO: 734, or a variant thereof having at least 70%, 75%, 80%, 85%, 90%, 95%, 98%, 99%, or 99.5% sequence identity thereto.
3. The peptide of embodiment 1 or 2, wherein the modified defensin peptide has at least 70%, 75%, 80%, 85%, 90%, 95%, 98%, 99%, or 99.5% sequence identity to SEQ ID NO: 536, 540, 544, 549, 553, 558, 563, 567, 571, 576, 581, 586, 591, 596, 601, 606, 610, 614, 618, 622, 626, 631, 635, 639, 643, 647, 651, 655, 660, 664, 668, 672, 677, 681, 686, 690, 695, 700, 704, 709, 717, 721, 726, 745, 748, 751, 754, or 756.
4. The peptide of embodiment 3, wherein the modified defensin peptide or modified defensin-like peptide comprises SEQ ID NO: 535, 536, 537, 539, 540, 541, 543, 544, 545, 547, 548, 549, 550, 552, 553, 554, 556, 557, 558, 559, 561, 562, 563, 564, 566, 567, 568, 570, 571, 572, 574, 575, 576, 577, 579, 580, 581, 582, 584, 585, 586, 587, 589, 590, 591, 592, 594, 595, 596, 597, 599, 600, 601, 602, 604, 605, 606, 607, 609, 610, 611, 613, 614, 615, 617, 618, 619, 621, 622, 623, 625, 626, 627, 629, 630, 631, 632, 634, 635, 636, 638, 639, 640, 642, 643, 644, 646, 647, 648, 650, 651, 652, 654, 655, 656, 658, 659, 660, 661, 663, 664, 665, 667, 668, 669, 671, 672, 673, 675, 676, 677, 678, 680, 681, 682, 684, 685, 686, 687, 689, 690, 691, 693, 694, 695, 696, 698, 699, 700, 701, 703, 704, 705, 707, 708, 709, 710, 712, 713, 714, 716, 717, 718, 720, 721, 722, 724, 725, 726, 727, 728, 729, 730, 731, 732, 733, 734, 735, 736, 744, 745, 747, 748, 750, 751, 753, 754, 755, 756, or a variant thereof comprising a deletion of 1 to 10 N-terminal amino acid residues, a deletion of 1 to 10 C-terminal amino acid residues, and/or a conservative amino acid substitution of 1 to 10 amino acid residues, wherein the modified gamma-core consensus peptide is conserved in the variant.
5. A peptide comprising the amino acid sequence of a modified defensin peptide fragment wherein the wild-type gamma-core consensus peptide of GXCX3-9C or GXCX3-22C of the corresponding wild-type defensin peptide fragment is replaced by a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M) (F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), wherein the peptide further comprises a second C-terminal cysteine residue located C-terminal to the cysteine residue in the modified gamma-core consensus sequence, wherein the peptide has a net positive charge of at least 3 and a hydrophobic amino acid content at least 18%, and optionally wherein the peptide comprises, essentially consists, or consists of: (i) 30 amino acid residues or less; or (ii) 15, 16, or 17 to 30 amino acid residues.
6. The peptide of embodiment 5, wherein said peptide comprises a modified C-terminal fragment of a wild-type defensin peptide, said modification comprising the substitution of the wild-type gamma-core consensus peptide of GXCX3-9C or GXCX3-22C of the wild-type defensin C-terminal fragment with the modified gamma-core consensus peptide, optionally wherein said peptide contains one additional cysteine residue C-terminal to the modified gamma-core consensus peptide sequence, and optionally wherein the peptide comprises a disulfide bond between the two cysteine residues in the modified C-terminal fragment having the one additional cysteine residue.
7. The peptide of embodiment 6, wherein said peptide comprises, consists essentially of, or consists of SEQ ID NO: 537, 541, 545, 550, 554, 559, 564, 568, 572, 577, 582, 587, 592, 597, 602, 607, 611, 615, 619, 623, 627, 632, 636, 640, 644, 648, 652, 656, 661, 665, 669, 673, 678, 682, 687, 691, 696, 701, 705, 710, 714, 718, 722, 727, or a variant thereof comprising a conservative amino acid substitution of 1 to 2, 3, 4, or 5 amino acid residues, wherein the modified gamma-core consensus peptide is conserved in the variant.
8. A peptide comprising a C-terminal fragment of a defensin-like peptide, wherein said C-terminal fragment lacks 1 to 35 amino terminal amino acids of the corresponding wild-type defensin-like peptide and/or comprises a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M)(F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M).
9. A peptide comprising, consisting essentially of, or consisting of SEQ ID NO: 729, 731, 733, 735, 736, 828, 829, 830, 831, 832, or 833, a variant thereof having comprising a conservative amino acid substitution of 1 to 2, 3, 4, or 5 amino acid residues, or a variant thereof having at least 90% or 95% sequence identity thereto, wherein the gamma-core consensus peptide is conserved in the variants or wherein the gamma-core consensus peptide is replaced with a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M) (F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), optionally wherein the gamma-core consensus peptide comprises the peptide sequence GXCX3-9C or GXCX9-16C.
10. A composition comprising the peptide of any one of embodiments 1 to 9 and an agriculturally, pharmaceutically, or veterinary-practicably acceptable carrier, diluent, or excipient.
11. The composition of embodiment 10, wherein the peptide is provided at a concentration of about 0.1, 0.5, 1.0, or 5 μg/ml to about 1, 5, 20, 50, or 100 mg/ml or at a concentration of about 0.1, 0.5, 1.0, or 5 μg/gram to about 1, 5, 20, 50, or 100 mg/gram and optionally wherein the composition comprises a sodium salt at a concentration of at least 100 mM and/or a calcium salt at a concentration of at least 2 mM.
12. A method for preventing or reducing crop damage by a plant pathogenic microbe comprising the step of contacting a plant, a plant seed, or other part of said plant with an effective amount of the composition of embodiment 10.
13. The method of embodiment 12, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp., Magnaporthe sp., or Stenocarpella sp.
14. A medical device comprising the device and the composition of embodiment 4, wherein the device comprises at least one surface that is topically coated and/or impregnated with the composition.
15. The medical device of embodiment 14, wherein said device is a stent, a catheter, a contact lens, a condom, a patch, or a diaphragm.
16. A method for treating, preventing, or inhibiting a microbial infection in a subject in need thereof comprising administering to said subject an effective amount of the composition of embodiment 10.
17. The method of embodiment 16, wherein said administration comprises topical, enteral, parenteral, and/or intravenous introduction of the composition.
18. The method of embodiment 16, wherein the subject is a human, livestock, poultry, fish, or a companion animal.
19. The method of embodiment 16, wherein the microbial infection is of a mucosal membrane, eye, skin, and/or a nail and the composition is applied to the mucosal membrane, eye, skin, and/or nail.
20. The method of any one of embodiments 16 to 19, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.
21. The method of any one of embodiments 16 to 19, wherein the microbial infection is by an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, or Blastomyces species.
22. The method of any one of embodiments 16 to 19, wherein the microbial infection is by a Candida species and wherein the Candida species is Candida albicans (C. albicans), C. auris, C. glabrata, C. parapsilosis, C. tropicalis, or C. krusei.
23. The composition of embodiment 10 for use in a method of treating, preventing, or inhibiting microbial infection in a subject in need thereof.
24. The composition of embodiment 23, wherein the subject is a human, livestock, poultry, fish, or a companion animal.
25. A plant part that is at least partly coated with the composition of embodiment 10.
26. The plant part of embodiment 25, wherein the part is a seed and the seed is optionally a corn, soybean, wheat, rice, cotton, Brassica sp., or tomato seed.
27. The plant part of embodiment 25, wherein the plant part is a leaf, stem, fruit, vegetable, root, tuber, or flower.
28. A recombinant polynucleotide comprising a polynucleotide encoding a peptide comprising the peptide of any one of embodiments 1 to 9, wherein the polynucleotide encoding the peptide is operably linked to a polynucleotide comprising a promoter which is heterologous to the polynucleotide encoding the peptide.
29. A recombinant polynucleotide encoding a peptide comprising: (i) a defensin peptide comprising a long gamma-core consensus sequence GXCX16-22C, optionally wherein the defensin peptide comprises SEQ ID NO: 578, 608, 612, or a variant thereof having at least 70%, 75%, 80%, 85%, 90%, 95%, 98%, or 99% sequence identity to SEQ ID NO: 578, 608, or 612, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues; or (ii) a defensin or defensin-like peptide comprising a wild-type gamma-core consensus peptide GXCX3-9C, GXCX9-16C or GXCX3-22C, optionally wherein the defensin or defensin-like peptide comprises SEQ ID NO: 534, 538, 560, 565, 569, 573, 583, 598, 603, 616, 624, 628, 633, 645, 697, 702, 711, 715, 723, 728, 729, 730, 731, 732, 733, 734, 735, 736, or a variant thereof having at least 70%, 75%, 80%, 85%, 90%, 95%, 98%, or 99% sequence identity to SEQ ID NO: 534, 538, 560, 565, 569, 573, 583, 598, 603, 616, 624, 628, 633, 645, 697, 702, 711, 715, 723, 728, 729, 730, 731, 732, 733, 734, 735, 736, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues; wherein the polynucleotide encoding the peptide is operably linked to a polynucleotide comprising a promoter which is heterologous to the polynucleotide encoding the peptide
30. The recombinant polynucleotide of embodiment 28 or 29, wherein the recombinant polynucleotide further comprises a polynucleotide encoding: (i) a transit peptide, a vacuolar targeting peptide, and/or an endoplasmic reticulum targeting peptide; (ii) a plastid targeting peptide; and/or (iii) a polyadenylation or transcriptional termination signal, wherein the polynucleotides of (i), (ii), and/or (iii) are operably linked to the polynucleotide encoding the antimicrobial peptide.
31. The recombinant polynucleotide of embodiment 28, 29, or 30, wherein the polynucleotide encoding the peptide is inserted into a heterologous nuclear or plastid genome of a cell and operably linked to an endogenous promoter located in the heterologous nuclear or plastid genome.
32. A plant nuclear or plastid genome comprising a polynucleotide encoding a peptide comprising the peptide of embodiment 1 to 9, wherein the polynucleotide is heterologous to the nuclear or plastid genome and wherein the polynucleotide is operably linked to an endogenous promoter of the nuclear or plastid genome.
33. A cell comprising the recombinant polynucleotide of any one of embodiments 28, 29, 30, or 31, wherein the cell is optionally a bacterial, yeast, or plant cell.
34. A plant comprising the recombinant polynucleotide of any one of embodiments 28, 29, 30, or 31.
35. A plant part of the plant of embodiment 34, wherein the plant part comprises the recombinant polynucleotide, optionally wherein the plant part is a seed, stem, leaf, root, tuber, flower, vegetable, or fruit.
36. A method for producing plant seed that provides plants resistant to infection by a plant pathogenic microbe that comprises the steps of: (i) selfing or crossing the plant of embodiment 34; and (ii) harvesting seed that comprises the recombinant polynucleotide of the plant from the self or cross, thereby producing plant seed that provide plants resistant to infection by a plant pathogenic microbe.
37. A method for producing an antifungal peptide comprising: (i) culturing the cell of embodiment 33 under conditions wherein the peptide, defensin, or defensin-like peptide is expressed by the cell; and (ii) purifying the peptide, defensin, or defensin-like peptide from the culture.
38. The method of embodiment 37, wherein the cell is a yeast cell, optionally wherein the recombinant polynucleotide comprises a polynucleotide encoding a transit peptide which is operably linked to the polynucleotide encoding the peptide, defensin peptide, or defensin-like peptide and optionally wherein the peptide, defensin peptide, or defensin-like peptide is purified from the culture supernatant.
39. The method of embodiment 38, wherein the yeast cell is a Candida, Kluveromyces, Hansuela, Pichia, Saccharomyces, Schizosaccharomyces, or Yarrowia cell.
40. An isolated peptide comprising: (i) a defensin peptide comprising a long gamma-core consensus sequence GXCX16-22C, optionally wherein the defensin peptide comprises SEQ ID NO: 578, 608, 612, or a variant thereof having at least 70%, 75%, 80%, 85%, 90%, 95%, 98%, or 99% sequence identity to SEQ ID NO: 578, 608, or 612, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues; or (ii) a defensin or defensin-like peptide comprising a wild-type gamma-core consensus peptide GXCX3-9C, GXCX9-16C or GXCX3-22C, optionally wherein the defensin or defensin-like peptide comprises SEQ ID NO: 534, 538, 560, 565, 569, 573, 583, 598, 603, 616, 624, 628, 633, 645, 697, 702, 711, 715, 723, 728, 729, 730, 731, 732, 733, 734, 735, 736, or a variant thereof having at least 70%, 75%, 80%, 85%, 90%, 95%, 98%, or 99% sequence identity to SEQ ID NO: 534, 538, 560, 565, 569, 573, 583, 598, 603, 616, 624, 628, 633, 645, 697, 702, 711, 715, 723, 728, 729, 730, 731, 732, 733, 734, 735, 736, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues.
41. A composition comprising the peptide of embodiment 40 and an agriculturally, pharmaceutically, or veterinary-practicably acceptable carrier, diluent, or excipient.
42. A method for preventing or reducing crop damage by a plant pathogenic microbe comprising the step of contacting a plant, a plant seed, or other part of said plant with an effective amount of the composition of embodiment 41.
43. The method of embodiment 42, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp., Magnaporthe sp., Stenocarpella sp., or Zymoseptoria sp.
44. A plant or plant part which is at least partially coated with the composition of embodiment 41.
45. The plant or plant part of embodiment 44, wherein the plant part is a leaf, seed, or fruit.

### PUBLICATIONS CITED

Argos (1990) J Mol Biol. February 20; 211(4):943-58.
Broadley, M. R., Bowen, H. C., Cotterill, H. L., Hammond, J. P., Meacham, M. C., Mead, A., and White, P. J. (2003). Journal of Experimental Botany 54, 1431-1446
Buchko et al. (2018) Protein Science 27, 1611-1623.
Chang et al. (2015) Amino Acids 47, 579-587.
Chen et al., (2013) Adv Drug Deliv Rev.; 65(10): 1357-1369.
da Silva Conceicao, A., and Broekaert, W. F. (1999). Plant Defensins. In Pathogenesis-related proteins in plants, S. Muthukrishnan, ed (New York: CRC Press), pp. 247-260.
Frangois et al., Plant Physiology (2002) 128:1346-1358.
George R A, and Heringa (2002) J Protein Eng. 15(11):871-879.
Hanks, J. N., et al., (2005). Plant Mol Biol 58, 385-399.
Kerenga B K, et al., (2019) Front. Microbiol. 10:795. doi: 10.3389/fmicb.2019.00795
Kiedzierska, et al. (2008) Protein Expr Purif 60, 82-8.
Kingsman S M, et al., (1985) Biotechnol Genet Eng Rev. 3:377-416.
Kuddus et al. (2017). Biotechnol Prog 233:1520-1528. doi: 10.1002/btpr.Protein Science 2508.
Kumar, V. and Jain, M. (2015) J Exp Bot 66:47-57.
Lacerda et al, Frontiers in Microbio. (2014) 5(116): 1-10.
Lay, F. T., and Anderson, M. A. (2005) Curr Protein Pept Sci 6, 85-101.
Marques et al. (2008) J Appl Microbiol 106, 1640-1648.
Mottram et al., FEBS Lett. (1989) 258(2):211-215.
Murovec et al., Plant Biotechnol J. 2017 August; 15(8): 917-926.
Pazgier et al. (2006) Protein Expr Pur 49, 1-8.
Reiser J, et al., (1990) Adv Biochem Eng Biotechnol.; 43: 75-102.
Sagaram et al., (2011) PLOS ONE 6: el8550.
Sagaram et al., (2013) PLoS ONE, 8(12): e82485.
Shabala, S., and Pottosin, I. I. (2010) Signal. Commun. Plants 87-110.
Shafee et al. (2016) Mol. Biol. Evol. 33(9):2345-2356; doi:10.1093/molbev/msw106
Svitashev et al., (2015) Plant Physiology, 169 (2): 931-945.
Spelbrink, R. G., et al., (2004). Plant Physiol 135, 2055-2067.
Terras, F. R., et al., (1992). J Biol Chem 267, 15301-15309.
Thomma, B. P. H. J., Cammue, B. P. A., and Thevissen, K. (2002). Planta 216, 193-202.
Turner et al., (1993) Protein Eng. 6(1): 101-108.
Voytas, D. Annual Review of Plant Biology, Vol. 64:327-350, 2013.
Vasivarama and Kirti (2013a) Plant Cell Tiss Organ Cult, 115:309-319.
Vasivarama and Kirti (2013b) Funct Integr Genomics 13:435-443.
Vriens, K., et al. Molecules 2014, 19, 12280-12303; doi: 10.3390/molecules190812280.
White, P., and Karley, A. (2010). Potassium. In Cell Biology of Metals and Nutrients; Hell, R., Mendel, R. R., Eds.; Springer: Berlin/Heidelberg, Germany., 199-224

### EXAMPLES

### Example 1

Crude peptides (GMA4C-AC, GMA4C_V1, GMA4C_V2 and GMA4C_V3) were synthesized chemically by Biomatik Inc, Canada, and GMA4C_V4 and GMA4C_V5 by Alan Scientific Inc. (USA) and were further purified, using a linear gradient of acetonitrile/water mixture, in a C-18 reverse phase HPLC (Agilent Technologies, USA). HPLC fractions were lyophilized and resuspended in nuclease-free water. Concentration of each peptide was determined using the BCA assay performed according to the manufacturer's protocol (Thermo-Fisher Scientific, USA). Antifungal activity was determined in assays in SFM culture media. The SFM culture media comprises K₂HPO₄ (2.5 mM), MgSO₄ (50 μM), CaCl₂) (50 μM), FeSO₄ (5 μM), CoCl₂ (0.1 μM), CuSO₄ ((0.1 μM), Na₂MoO₄ (2 μM), H₃BO₃ (0.5 μM), KI (0.1 μM), ZnSO₄ (0.5 μM), MnSO₄ (0.1 μM), glucose (10 g/liter), asparagine (1 g/liter), methionine (20 mg/liter), myo-inositol (2 mg/liter), biotin (0.2 mg/liter), thiamine-HCL (1 mg/liter), and pyridoxine-HCL (0.2 mg/liter), pH 7.0.)

TABLE 2
Antifungal Activity of Peptides

| | SEQ ID | B. cinerea | F. graminearum | F. oxysporum | P. capsici |
|---|---|---|---|---|---|
| Peptide | NO. | MIC (μM) | MIC (μM) | MIC (μuM) | MIC (μM) |
| GMA4C_AC ¹ | 757 | 1.5-3.0 | 3 | 6 | 1.5 |
| GMA4C_V1A | 758 | 1.5 | 3 | 6 | 3 |
| GMA4C_V2A | 759 | 3 | 3-6 | — | — |
| GMA4C_V3A | 760 | 1.5 | 3 | — | — |
| GMA4C_V4 | 761 | 1.5 | 3 | — | — |
| Non-amidated | | | | | |
| GMA4C_V5 | 763 | 1.5 | 3 | — | — |
| Non-amidated | | | | | |
| GMA4C_V6A | 765 | 0.75 | 1.5 | — | — |

¹ C-terminus is amidated.

TABLE 3
Comparison of GMA4C wild-type and variant peptides

| | | MIC | | | |
|---|---|---|---|---|---|
| | | Values | | | |
| | | (μM) | MIC Values | | |
| | | against | (μM) against | | |
| Peptide | SEQ ID | B. | F. | Salt | |
| Name | NO: | cinerea | graminearum | tolerance | Comments |
| GMA4C_AC | 757 | 3 | 6 | + | Chemical synthesis, |
| | | | | | amidated |
| GMA4C_V1A | 758 | 1.5 | 3 | ++ | Chemical synthesis, |
| | | | | | amidated |
| GMA4C_V2A | 759 | 3 | 6 | ++ | Chemical synthesis, |
| | | | | | amidated |
| GMA4C_V3A | 760 | 3 | 3 | ++ | Chemical synthesis, |
| | | | | | amidated |
| GMA4C_V4A | 761 | 1.5 | 1.5 | +++ | Chemical synthesis, |
| | | | | | amidated |
| GMA4C_V5A | 763 | 3 | 3 | + | Chemical synthesis, |
| | | | | | amidated |
| GMA4C_V6A | 765 | 0.75 | 1.5 | +++ | Chemical synthesis, |
| | | | | | amidated |

Antifungal activity in vitro in low cationic conditions: The GMA4C variants GMA4C_VIA, GMA4C_V3A, GMA4C_V4A, and GMA4C_V5A exhibit antifungal activity equivalent to the wild-type GMA4C_AC control in vitro against B. cinerea, F. graminearum, F. oxysporum and P. capsici. The GMA4C variant GMA4C_V2A exhibit a two-fold reduction in antifungal activity relative to the wild-type GMA4C_AC control in vitro against B. cinerea and F. graminearum. GMA4C_GMA4C_V6A exhibits a four-fold increase in antifungal activity relative to the wild-type GMA4C_AC control in vitro against B. cinerea.

Antifungal activity in planta. When applied on detached Nicotiana benthamiana leaves, GMA4C_VIA and GMA4C_V3A are more potent than GMA4C_AC or GMA4C_V2A in reducing gray mold disease symptoms caused by B. cinerea as shown in FIGS. 1A and 1B. When applied on tomato leaves, GMA4C_VIA is more effective in reducing disease symptoms caused by P. capsici than GMA4C_AC.

Antifungal activity in vitro in presence of cations. When tested for antifungal activity against B. cinerea in the presence of 100 mM NaCl, 100 mM KCl, or 2 mM CaCl₂), all of the peptides including GMA4C_AC retain their antifungal activity in presence of 100 mM NaCl or 100 mM KCl. However, GMA4C_VIA and GMA4C_V4A have 2-fold more potent activity than GMA4C_AC. In presence of 2 mM CaCl₂), only GMA4C_VIA and GMA4C_V4A inhibit fungal growth at 6 μM, whereas other peptides such as plant defensins MtDef4 or OeDef1 show little or no activity at this concentration.

TABLE 4
GMA4C and its variants retain antifungal activity against B. cinerea in presence of salts.

| | | | SFM + 100 mM | SFM + 100 mM | SFM + 2 mM |
|---|---|---|---|---|---|
| | SEQ ID | SFM MIC | NaCl | KCl | CaCl₂ |
| Peptide | NO: | (μM) ¹ | MIC (μM) ¹ | MIC (μM) ¹ | MIC (μM) ¹ |
| GMA4C_AC ¹ | 757 | 3 | 3 | 6 | >6 (a few |
| | | | | | germinated at |
| | | | | | 6) |
| GMA4C_V1A- | 758 | 3 | 1.5 | 3 | 6 |
| GMA4C_V2A- | 759 | 6 | 3 | 6 | >6 |
| GMA4C_V3A- | 760 | 3 | 1.5-3 | >6 | >6 |
| GMA4C_V4A- | 761 | 1.5-3 | 1.5 | 1.5 | 3-6 |
| GMA4C_V4 | 762 | 3 | 3 | 3 | 6 |
| Non-amidated | | | | | |
| GMA4C_V5A | 763 | 6 | 6 | >6 | >6 |
| GMA4C_V5 | 764 | 6 | 6 | >6 | >6 (very |
| GMA4C_V6A | 765 | 1.5 | 0.75-1.5 | 1.5-3 | 3-6 |

¹ The Minimal Inhibitory Concentration (MIC) is the concentration of the peptide at which there is no significant growth of the microorganism relative to the growth of the microorganism in growth medium lacking the compound, protein, or peptide.

Antifungal activity against human fungal pathogens: GMA4C_VIA and GMA4C_V3A show antifungal activity against C. auris, and C. glabrata in the RPMI medium rich in cations, but the parental MtDef4 defensin does not show activity.

TABLE 5
Composition of Roswell Park Memorial Institute
1640 culture medium (RPMI media; with glutamine
and phenol red but without bicarbonate)

| Constituent | Water, g/L | Constituent | Water, g/L |
|---|---|---|---|
| L-arginine (free base) | 0.2 | Biotin | 0.0002 |
| L-asparagine (anhydrous) | 0.05 | D-pantothenic acid | 0.00025 |
| L-aspartic acid | 0.02 | Choline chloride | 0.003 |
| L-cystine * 2HC1 | 0.0652 | Folic acid | 0.001 |
| L-glutamic acid | 0.02 | Myo-inositol | 0.035 |
| L-glutamine | 0.3 | Niacinamide | 0.001 |
| Glycine | 0.01 | PABA | 0.001 |
| L-histidine (free base) | 0.015 | Pyridoxine HCI | 0.001 |
| L-hydroxyproline | 0.02 | Riboflavin | 0.0002 |
| L-isoleucine | 0.05 | Thiamine HCI | 0.001 |
| L-leucine | 0.05 | Vitamin B₁₂ | 0.000005 |
| L-lysine * HCI | 0.04 | Calcium Nitrate H₂O | 0.01 |
| L-methionine | 0.015 | Potassium chloride | 0.4 |
| L-phenylalanine | 0.015 | Magnesium sulfate | 0.04884 |
| | | (anhydrous) | |
| L-proline | 0.02 | Sodium chloride | 6 |
| L-serine | 0.03 | Sodium phosphate, | 0.8 |
| | | dibasic (anhydrous) | |
| L-threonine | 0.02 | D-glucose | 2 |
| L-tryptophan | 0.005 | Glutathione, reduced | 0.001 |
| L-tyrosine * 2Na | 0.02883 | Phenol red, Na | 0.0053 |
| L-valine | 0.02 | | |

TABLE 6
GMA4C_V1A and GMA4C_V3A exhibit antifungal activity in vitro
in the RPMI medium against Candida auris and C. glabrata.

| | | | Candida |
|---|---|---|---|
| | Candida albicans | Candida auris | glabrata |
| Peptide | MIC (μg/ml) ¹ | MIC (μg/ml) ¹ | MIC (μg/ml) ¹ |
| MtDef4 | >46.9 | 46.9 | >46.9 |
| GMA4C_V1A | 46.9 | 11.9 | 11.9 |
| GMA4C_V3A | >46.9 | 23.9 | 23.9 |

¹ The Minimal Inhibitory Concentration (MIC) is the concentration of the protein or peptide at which there is no significant growth of the microorganism relative to the growth of the microorganism in growth medium lacking the compound, protein, or peptide.

### Example 2. Activity of GMA4C-Variants Against Pathogenic Microbes

Testing for antifungal activity used half-strength potato dextrose broth for peptides and RPMI for comparator antifungals fluconazole and voriconazole. CLSI M27 and M38 methodologies used for to measure MICs. Minimal Inhibitory Concentration (MIC) is the concentration of the compound, protein, or peptide at which there is no significant growth of the microorganism relative to the growth of the microorganism in growth medium lacking the compound, protein, or peptide.

All testing was performed in RPMI buffered with 0.165M MOPS. The concentration range for peptides in potato dextrose broth was 0.06-2 mcg/ml, the concentration range for fluconazole was 0.125-64 mcg/ml, and the concentration range for voriconazole was 0.03-16 mcg/ml. MICs were determined at 24-72 hours.

TABLE 7
Formula for Potato Dextrose Broth
Potato Dextrose Broth

| | |
|---|---|
| Value | Ingredients and conditions |

| | | |
|---|---|---|
| 1000 | ml | water |

| | |
|---|---|
| 4 g (from 200 g infused | Potatoes (sliced, unpeeled, |
| potato) | washed) |

| | | |
|---|---|---|
| 20 | g | Glucose |

| | |
|---|---|
| pH 5.6 | Final pH |

| | | |
|---|---|---|
| 25 | C | Temperature |

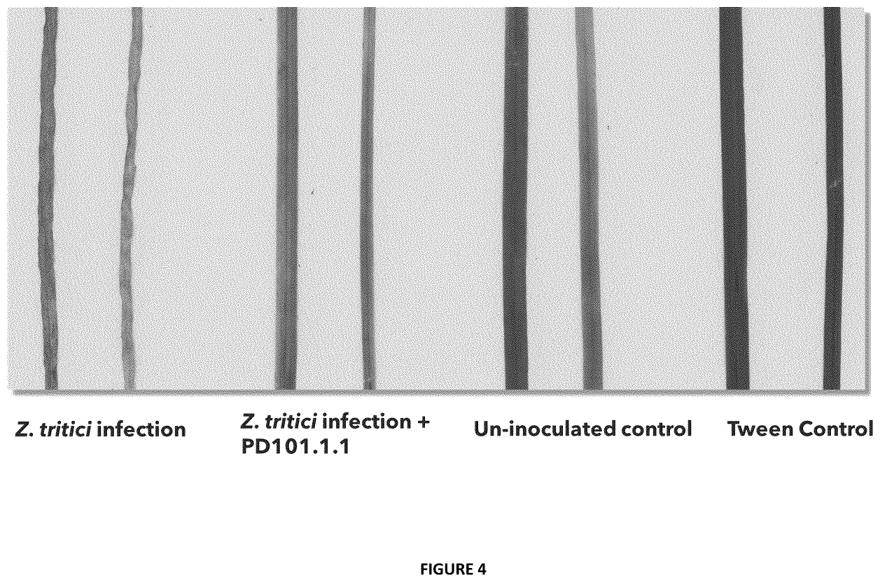

TABLE 8
MIC values (μg/ml) for GMA4C variants. The complete table with
data for fluconazole and voriconazole are provided in FIG. 4.

| | | GMA4C_V1A | GMA4C_V2A | GMA4C_V4A | GMA4C_V5A |
|---|---|---|---|---|---|
| Species | Isolate | (μg/ml) | (μg/ml) | (μg/ml) | (μg/ml) |
| (isolate) | No. | 100% | 100% | 100% | 100% |
| C. | ATCC | 4 | 8 | 8 | 8 |
| parapsilosis | 22019 | | | | |
| C. krusei | ATCC | 8 | 16 | 8 | 8 |
| | 6258 | | | | |
| P. variotii | MYA-363 | 4 | 8 | 8 | 4 |
| Candida | SC5314 | 8 | 16 | 8 | 16 |
| albicans | ATCC | 8 | 16 | 8 | 16 |
| | 90028 | | | | |
| | CA3 | 8 | 16 | 8 | 8 |
| Candida | DI17-47 | 8 | 16 | 8 | 16 |
| auris | DI17-48 | 8 | 16 | 8 | 16 |
| | DI17-46 | 8 | 16 | 8 | 16 |
| Aspergillus | AF293 | 32 | 32 | 16 | 32 |
| fumigatus | DI15-106 | >32 | >32 | 16 | >32 |
| | DI15-116 | >32 | >32 | 32 | >32 |
| Fusarium | F1 (F. | 4 | 8 | 8 | 8 |
| | oxysporum) | | | | |
| | F2 (F. | 4 | 16 | 8 | 16 |
| | oxysporum) | | | | |
| | F4 (F. | 4 | 8 | 8 | 8 |
| | solani) | | | | |
| Coccidioides | Cocci 1 | 8 | 8 | 4 | 2 |
| sp. | Cocci2 | 4 | 16 | 16 | 16 |
| | DI17-143 | 8 | 16 | 16 | 16 |

TABLE 9
Antifungal activity of GMA4C wild-type and its variants against plant fungal
pathogens in SFM.

| SEQ | | | MIC (μM) | MIC (μM) |
|---|---|---|---|---|
| ID | | | Botrytis | Fusarium |
| NO: | Peptide | Sequence | cinerea | graminearum |
| 757 | GMA4C_AC | GGRCRGFRRRCFCTTHC-NH₂ | 3 | 3 |
| 758 | GMA4C_V1A | GGRCKGFRRRCFCTRIC-NH₂ | 3 | 3 |
| 759 | GMA4C_V2A | GGRCRGFRRRCFCTRIC-NH₂ | 6 | 3-6 |
| 760 | GMA4C_V3A¹ | GGRCRGFRRRVFVTRIC-NH₂ | 3 | 3 |
| 761 | GMA4C_V4A | FGRCRGFRRRCFCWRWC-NH₂ | 1.5 | — |
| 763 | GMA4C_V5A² | FG(Dab)C(Dab)GF(Dab)(Dab) | 6 | — |
| | | (Dab)CFCW(Dab)WC-NH₂ | | |
| 766 | GMA4AC | GRCRGFRRRCFCTTHC | 6 | 6 |
| 762 | GMA4C_V4 | FGRCRGFRRRCFCWRWC | 3 | 3 |
| 764 | GMA4C_V5 | FG(Dab)C(Dab)GF(Dab)(Dab) | 6 | 3 |
| | | (Dab)CFCW(Dab)WC | | |
| 765 | GMA4C_V6 | GGRCKGFRRRWFWTRIC-NH₂ | 0.75 | 1.5 |

TABLE 10
Antifungal activity of GMA4C wild-type and its variants
against plant fungal pathogens in SFM + salts

| | | SFM + | SFM + | |
|---|---|---|---|---|
| | | 100 mM | 100 mM | SFM + 2 mM |
| Peptide | SFM MIC | NaCl | KCl | CaCl₂ |
| (SEQ ID NO) | (μM) | MIC (μM) | MIC (μM) | MIC (μM) |
| GMA4C_AC1-2 | 3 | 3 | 6 | >6 (a |
| (SEQ ID NO: | | | | few germinated |
| 757) | | | | at 6) |
| GMA4C_V1A-3 | 3 | 1.5 | 3 | 6 |
| (SEQ ID NO: | | | | |
| 758) | | | | |
| GMA4C_V2A-4 | 6 | 3 | 6 | >6 |
| (SEQ ID NO: | | | | |
| 759) | | | | |
| GMA4C_V3A-5 | 3 | 1.5-3 | >6 | >6 |
| (SEQ ID NO: | | | | |
| 760) | | | | |
| GMA4C_V4A-6 | 1.5-3 | 1.5 | 1.5 | 3-6 |
| (SEQ ID NO: | | | | |
| 761) | | | | |
| GMA4C_V4 | 3 | 3 | 3 | 6 |
| Non-amidated-11 | | | | |
| (SEQ ID NO: | | | | |
| 762) | | | | |
| GMA4C_V5A | 6 | 6 | >6 | >6 |
| (SEQ ID NO: | | | | |
| 763) | | | | |
| GMA4C_V5 | 6 | 6 | >6 | >6 (very |
| Non-amidated 12 | | | | sensitive) |
| (SEQ ID | | | | |
| NO: 764) | | | | |
| GMA4C_V6A - | 1.5 | 0.75-1.5 | 1.5-3 | 3-6 |
| 13 (SEQ ID NO: | | | | |
| 765) | | | | |

TABLE 11
Antifungal activity of peptides against human yeast
pathogens in half-strength potato dextrose broth

| | Candida | Candida | Candida |
|---|---|---|---|
| | albicans | auris | glabrata |
| | (33795) | (38883) | (38827) |
| Peptide | MIC (μM) | MIC (μM) | MIC (μM) |
| MtDef4 ¹ | 1.1 | 1.1 | 0.55 |
| GMA4C_V1A | 11.2 | 11.2 | 11.2 |
| (SEQ ID NO: 758) | | | |
| GMA4C_V3A | 2.8 | 5.6 | 2.8 |
| (SEQ ID NO: 760) | | | |

¹ Full Length MtDef4 protein is described in US. Pat. No. 7,825,297.

All pathogen isolates used in this assay are resistant to an antifungal drug Fluconazole. Antifungal assays were conducted in half-strength potato dextrose broth.

TABLE 12
Synergistic (or Additive) Effects of
GMA4C_v3 or MtDef4/Fluconazole Combination

| | GMA4C_V3A (SEQ ID NO: 760)/Fluconazole | | | |
|---|---|---|---|---|
| | | MIC in | | |
| | MIC alone | combination | | |
| | μg/ml | ug/ml | | |
| | (μM) | (μM) | FICI¹ | Interpretation |
| C. albicans | 11.7/64 | 2.9/8 | 0.375 | Synergistic |
| | (4.2/206) | (1.4/25.8) | | |
| C. glabrata | 11.7/64 | 5.9/8 | 0.625 | Additive |
| | (4.2/206) | (2.8/25.8) | | |

Full Length MtDef4² Protein/Fluconazole

| | | MIC in | | |
|---|---|---|---|---|
| | MIC alone | combination | | |
| | μg/ml | μg/ml | | |
| | (μM) | (μM) | FICI ¹ | Interpretation |
| C. albicans | 5.9/64 | 2.9/2 | 0.53 | Additive |
| | (1.1/206) | (0.55/6.45) | | |
| C. glabrata | 2.9/64 | 1.5/64 | 1.5 | No |
| | (1.1/206) | (0.28/206) | | difference |

Conclusion: GMA4C_v3 shows synergistic enhancement of antifungal activity against drug resistant C. albicans in combination with Fluconazole
¹ FICI is Fractional Inhibitory Concentration Index. It is calculated as MIC_{A} combination/MIC alone + MIC_{B} combination/MIC_{B} alone where MIC_{A} combination is the MIC of agent A in combination and MIC_{A} alone is the MIC of agent A alone). Agent A is peptide (GMAC_V3A)
and Agent B is Fluconazole in top table. Agent A is full length MtDef4 protein and Agent B is Fluconazole in fluconazole in bottom table.
² Full Length MtDef4 protein is described in US. Pat. No. 7,825,297, incorporated herein by reference in its entirety.

TABLE 13
Antifungal activity of peptides (MIC in μM)
against human nail pathogens (Tricophyton spp.)
and Aspergillosis (Aspergillus fumigatus).

| | Tricophyton | Tricophyton | Aspergillus |
|---|---|---|---|
| Peptide | rubrum | metagrophytes | fumigatus |
| (SEQ ID NO:) | (23014) | (26103) | (36843) |
| MtDef4 ¹ | 2.2 | 1.1 | >8.9 |
| GMA4C_V1A | 11.2 | 11.2 | 22.4 |
| (SEQ ID NO: 758) | | | |
| GMA4C_V3A | 5.6 | 5.6 | 11.2 |
| (SEQ ID NO: 760) | | | |
| OeDef1 ² | 3.8 | 3.8 | >7.6 |
| OeDef1_V3 | 4.0 | 4.0 | >7.9 |
| (SEQ ID NO: 769) | | | |

¹ Full Length MtDef4 protein is described in US. Pat. No. 7,825,297.
² Full length OeDef1 defensin protein is described in WO 2020/146373.

TABLE 14
Antifungal activity of GMA4C variants (MIC in μM) against
Candida spp. and Aspergillosis (Aspergillus fumigatus).

| | | GMA4C_V1A | GMA4C_V2A | GMA4C_V4 | GMA4C_V5 |
|---|---|---|---|---|---|
| | | (SEQ ID NO: | (SEQ ID NO: | (SEQ ID NO: | (SEQ ID |
| Species | Isolate | 758) | 759) | 762) | NO: 764) |
| Candida | SC5314 | 3.8 | 7.6 | 3.8 | 7.6 |
| albicans | | | | | |
| | ATCC | 3.8 | 7.6 | 3.8 | 7.6 |
| | 90028 | | | | |
| | CA3 | 3.8 | 7.6 | 3.8 | 3.8 |
| Candida | ATCC | 1.9 | 3.8 | 3.8 | 3.8 |
| parapsilosis | 22019 | | | | |
| Candida | DI17-47 | 3.8 | 7.6 | 3.8 | 7.6 |
| auris | | | | | |
| | DI17-48 | 3.8 | 7.6 | 3.8 | 7.6 |
| | DI17-46 | 3.8 | 7.6 | 3.8 | 7.6 |
| Aspergillus | AF293 | 15.2 | 15.2 | 7.6 | 15.2 |
| fumigatus | | | | | |
| | DI15-106 | >15.2 | >15.2 | 7.6 | >15.2 |
| | DI15-116 | >15.2 | >15.2 | 15.2 | >15.2 |
| Fusarium | F1 (F. | 3.8 | 3.8 | 3.8 | 3.8 |
| | oxysporum) | | | | |
| | F2 (F. | 3.8 | 7.6 | 3.8 | 7.6 |
| | oxysporum) | | | | |
| | F4 (F. | 3.8 | 3.8 | 3.8 | 3.8 |
| | solani) | | | | |
| Coccidioides | Cocci1 | 3.8 | 3.8 | 1.9 | 0.9 |
| sp. | | | | | |
| | Cocci2 | 1.9 | 7.6 | 7.6 | 7.6 |
| | DI17-143 | 1.9 | 7.6 | 7.6 | 7.6 |

### Example 3. Antibacterial Activity of GMA4C_VIA Against Human Bacterial Pathogens

Salmonella Typhimurium var. Copenhagen, Enterotoxigenic E. coli—F4 and Listeria monocytogens (F5244) strains were grown overnight on an LB agar plate at 37_C. A small number of bacteria was scraped from the plate and added to Mueller-Hinton (MH) growth media and grown to log phase. Cells were diluted to 1-3×10⁶ CFU/mL and 50 ul were added to each well in a polypropylene 96-well plate. Synthesized peptide GMA4C_VIA was diluted into 0.2% BSA, 0.01% acetic acid solution and added to the concentration of 0.2, 0.4, 0.80, 1.6, 3.25, 7.5, 15, 30 and 60 and 120 μM. Then, 50 μl of each peptide solution was added to 50 μl of bacterial cells. Plates were parafilmed and incubated at 37 °C overnight. The concentrations at which bacterial growth inhibition was determined based on the OD₆₀₀ nm value at each concentration relative to the OD₆₀₀ nm value of MH medium alone and that of bacterial growth in MH medium without any peptide. The Minimal Inhibitory Concentration (MIC) is the concentration of the peptide at which there is no significant growth of the microorganism relative to the growth of the microorganism in growth medium lacking the peptide.

TABLE 15
Antibacterial activity of GMA4C_V1A (SEQ ID NO: 758)
Concentrations of GMA4C_V1A (in μM) at which growth
inhibition of each bacterial pathogen was observed

| | S. Typhimurium var. | Enterotoxigenic | L. monocytogenes |
|---|---|---|---|
| Peptide | Copenhagen | E. coli-F4 | F5244 |
| GMA4C_V1A | 15-60 | 7.5-60 | 15-30 |

### Example 4. Antimicrobial Activity of Defensin Peptide Fragments

Crude chemically synthesized defensin-derived peptides with 80-85% purity were obtained from Biomatik Inc, Canada or from Alan Scientific Inc., USA. Each peptide was further purified using a C-18 reverse phase HPLC (Agilent, Singapore). HPLC fractions containing the peptide were lyophilized and resuspended in nuclease-free water. Concentrations were determined using BCA assays using manufacturer's protocol (Thermo-Fisher Scientific) for their accurate quantification.

The fungal strains of Botrytis cinerea T-4, Alternaria alternata, Cercospora sojina and, Colletotrichum gloeosporioides were each cultured in their respective normal growth medium shown in Table 16. Fungal spores were harvested by flooding the fungal growth plates with sterile water. The spore suspension was filtered through two layers of Miracloth, centrifuged at 13,600 rpm for 1 min, washed, and re-suspended in low-salt Synthetic Fungal Medium (SFM) (U.S. Pat. No. 6,316,407). The spore suspension was adjusted to the desired spore count using a hemocytometer.

TABLE 16

| | Medium for spore | Culture |
|---|---|---|
| Strains | production | conditions |
| B. cinerea | 20% V8 | 7-25 days, 25° C. |
| Alternaria | 10% Potato-Dextrose Agar | 7-15 days, 25° C. |
| alternata | | |
| Cercospora | 20% V8 | 12 hr light/12 hr |
| sojina | | dark at 25° C. |
| Colletotrichum | 10% Potato-Dextrose Agar | 7-15 days, 25° C. |
| gloeosporioides | | |

The antifungal activity of truncated defensin-derived peptides and their variants against B. cinerea, A. alternata, C. sojina and C. gloeosporioides fungal pathogens was determined spectrophotometrically using a 96-well plate assay (Sagaram et al., (2011) PLoS ONE 6: e18550. doi: 10.1371/journal.pone.0018550). Forty-five microliter of peptide at concentrations of 0.375, 0.75, 1.5, 3, 12 μM was added to each well of the microtiter plate containing 45 μL of (~10⁵ B. cinerea spores/ml) spore suspension. The quantitative fungal growth inhibition was determined by measuring the absorbance at 595 nm using a (Tecan Infinite M200 ProTecan Systems Inc., San Jose, CA) microplate reader after 48 h. Fungal cell viability was determined using the resazurin cell viability assay (Chadha and Kale, (2015) Lett Appl Microbiol 61, 238-244, Li et al., (2019) Mol Plant Microbe Interact 32, 1649-1664). After incubation of the pathogen/peptide mixture for 48 h, 10 μl of 0.1% resazurin solution was added to each well and re-incubated overnight. A change in color of the resazurin from blue to pink or colorless indicated the presence of live fungal cells. The MIC value of each peptide was determined as the minimal concentration of peptide at which no change in blue color was observed. MIC values of defensin-derived peptides and their variants were also determined in SFM and SFM supplemented with 100 mM NaCl, 100 mM or 2 mM CaCl₂) as described above.

The semi-in planta antifungal activity of each defensin-derived peptide and its variants against B. cinerea was determined using the detached leaves of N. benthamiana Nb1 as described previously (Li et al., (2019) Mol Plant Microbe Interact 32, 1649-1664; Velivelli et al., (2020) Proceedings of the National Academy of Sciences. 117, 16043-16054). Each peptide was tested at concentrations of 1.5 μM, 3 μM and 6 μM. Following incubation of each peptide/fungal spore mixture at room temperature for 48 h, leaves were photographed in white-light. High-resolution fluorescence images were also taken using CropReporter (PhenoVation, Wageningen, Netherlands). These images depicted the calculated F_{V}/F_{M} (maximum quantum yield of photosystem II) values of diseased area affected by B. cinerea infection. Colors in the images show five different classes ranging from class I to class V (0.000 to 0.700) depicting varying degrees of tissue damage. Green color in each image represents class V in 0.600 to ≥0.700 range depicting healthy area on leaf surface. In contrast, red color represents class I in the 0.000 to 0.160 range and depicts severely damage or diseased leaf surface.

The primary amino acid sequences, length, net charge and percentage of hydrophobic amino acids of defensin-derived peptides are shown in Table 17. These peptides are derived from plant defensins OeDef1, MtDef4, MsDefl and MtDef5A. Amino acid substitutions in the wild-type sequence of each peptide were made to increase the net charge and hydrophobicity. In addition, a disulfide bond was introduced into specific variants to make them pseudo-cyclic. Peptides that can form a single disulfide bond and a pseudo-cyclic peptide are shown as “+” in the “Disulfide Bond” column of Table 17. All peptides also carry a carboxy-terminal amide group.

TABLE 17
Amino acid sequences, length, net charge and % hydrophobic amino acids of
defensin-derived peptides. The presence of a disulfide bond is indicated where
necessary.

| | | SEQ | | | % |
|---|---|---|---|---|---|
| | | ID | | Net | Hydrophob. |
| Peptide | Sequence (disulfide presence) | NO | Length | Charge | amino acids |
| GMAOe1C | GACLKNRHSKHYGCYCYYRHCY- | 768 | 22 | 4.5 | 32 |
| (WT) | NH₂ | | | | |
| GMAOe1C_V3 | GACLKNRHSKHYGWFWYYRHCY- | 769 | 22 | 4.5 | 41 |
| | NH₂ (disulfide +) | | | | |
| GMAOe1C_V4 | GACLKNRHSKHYGFFWYYRHCY- | 770 | 22 | 4.5 | 41 |
| | NH₂ (disulfide +) | | | | |
| GMA4AC | GRCRGFRRRCFCTTHC-NH₂ | 771 | | | |
| GMA4C_V9 | GGRCKGFLRRFWFTRIC-NH₂ | 772 | 17 | 5 | 35 |
| | (disulfide +) | | | | |
| GMA4C_V10 | GGRCKGFRRRWYWTRIC-NH₂ | 773 | 17 | 6 | 29 |
| | (disulfide +) | | | | |
| GMA1C_V1 | SGRCRILFRCFCTKNC-NH₂ | 774 | 16 | 4 | 25 |
| GMA1C_V2 | SGRCRILFRWFWTKNC-NH₂ | 775 | 16 | 4 | 37 |
| | (disulfide +) | | | | |
| GMA1C_V3 | SGRCRILFRWYFTKNC-NH₂ | 776 | 16 | 4 | 37 |
| | (disulfide +) | | | | |
| GMA5CA_WT | GACHRQGFGFACFCYKKC-NH₂ | 777 | 18 | 3.5 | 28 |
| GMA5CA_V3 | GACHRQGFGFAWFWYKKC-NH₂ | 778 | 18 | 3.5 | 39 |
| | (disulfide +) | | | | |
| GMA5CA_V4 | GACHRQGFGFAFFFYKKC-NH₂ | 779 | 18 | 3.5 | 39 |
| | (disulfide +) | | | | |

The minimal inhibitory concentration (MIC) value of each defensin-derived peptide and its variants was determined (Table 18). It has been hypothesized that the presence of cations significantly weakens the electrostatic interactions between a positively charged defensin and negatively charged fungal membranes (Chu et al., (2013) Antimicrobial Agents and Chemotherapy 57:4050-4052). We therefore determined the antifungal activity of each peptide against B. cinerea in SFM supplemented with 100 mM NaCl, 100 mM KCl or 2 mM CaCl₂ (Table 18).

TABLE 18
MIC values of defensin-derived peptides against Botrytis cinerea

| | | | MIC (μM) | MIC (μM) | MIC (μM) |
|---|---|---|---|---|---|
| | SEQ | | (SFM + | (SFM + | (SFM + |
| | ID | MIC (μM) | 100 mM | 100 mM | 2 mM |
| Peptide | NO | SFM | NaCl) | KCl) | CaCl₂) |
| GMAOe1C_WT | 768 | 3 | 3-6 | 6-12 | >12 |
| GMAOe1C_V3 | 769 | 1.5 | 2-3 | 6-12 | 3-6 |
| GMAOe1C_V4 | 770 | 1.5 | 6 | 6-12 | 6-12 |
| GMA4AC | 771 | 3 | ND¹ | ND | ND |
| GMA4C_V9 | 772 | 3 | 3 | 6-12 | 12 |
| GMA4C_V10 | 773 | 3 | 3 | 3-6 | 3 |
| GMA1C | 774 | >12 | >12 | >12 | >12 |
| GMA1C_V1 | 775 | >6 | >6 | >12 | — |
| GMA1C_V2 | 776 | 1.5-3 | 3 | >12 | 3 |
| GMA1C_V3 | 777 | 1.5 | 3 | >12 | 6 |
| GMA5CA_V3 | 779 | 3 | >12 | >12 | >12 |
| GMA5CA_V4 | 768 | 3 | >12 | >12 | >12 |

¹ND is not determined.

MIC values of defensin-derived peptides were also tested against Alternaria alternata, Cercospora sojina, and Colletotrichum gloeosporioides (Table 19).

TABLE 19
In vitro antifungal activity of antifungal peptides
against Alternaria alternata, Cercospora sojina
and Colletotrichum gloeosporioides

| | SEQ | Alternaria | Cercospora | Colletotrichum |
|---|---|---|---|---|
| Peptide | ID NO | alternata | sojina | gloeosporioides |
| GMA4AC_WT | 771 | >12 | >12 | >12 |
| GMA 4C_V9 | 772 | >12 | 6 | >12 |
| GMA 4C_V10 | 773 | >12 | 6 | >12 |
| GMA5CA_WT | 777 | 6 | 12 | >12 |
| GMA5CA_V3 | 778 | 3 | 6 | >12 |
| GMA5CA_V4 | 779 | 3 | 6 | >12 |

Semi-in planta antifungal activity of GMA4C_V9, GMA4C_V10, GMAOe1C_WT, GMAOe1C_V3, GMAOe1C_V4, GMA1C_V1 and GMA1C_V2 peptides against B. cinerea was determined by using the detached leaves of N. benthamiana. Each peptide at concentrations of 1.5 μM, 3 μM and 6 μM was applied on leaf as drops and freshly prepared conidial inoculum was applied immediately to each drop of the peptide. Leaves were assessed for attenuation of gray mold symptoms after 48 h of inoculation relative to no peptide controls by measuring lesion sizes. GMA4C_V9 and GMA4C_V10 peptides are both effective in reducing gray mold disease symptoms. However, at low concentrations of 1.5 μM and 3 μM, GMA4C_V10 is more effective in reducing the symptoms of gray mold than GMA4C_V9.

A drop inoculation assay was also performed to test the antifungal activity of GMAOe1C_WT, GMAOe1C_V1, GMAOe1C_V2. The results revealed that GMAOe1C_V1 and GMAOe1C_V2 at concentration of 3 and 6 μM completely abrogated gray mold symptoms, but GMAOe1C_WT was only effective at 6 μM. At a concentration of 1.5 μM, GMAOe1C_V3 is more effective than GMAOe1C_V4 or GMAOe1C_WT.

A drop inoculation assay was also performed to test the antifungal activity of GMA1C_V1 and GMA1C_V2. GMA1C_V2 at 3 μM and 6 μM completely abrogated gray mold symptoms. GMA1C_V1 failed to reduce disease symptoms at these concentrations. At a concentration of 1.5 μM, GMA1C_V2 was more effective than GMA1C_V1.

The results above indicate that the panel of modifications (e.g., amino acid substitutions) introduced into these truncated defensin-derived peptides confer greater antifungal activity than the wild-type truncated peptides.

### Example 5. Recombinant Expression and Purification of Defensin or Defensin-Like Peptides

The codon-optimized synthetic genes encoding defensin or defensin-like peptides were custom synthesized by GenScript (Piscataway, NJ). The synthetic genes were cloned between the XhoI and XbaI sites of the pPICZαA vector in frame with the α-factor secretion signal sequence for expression in Pichia pastoris X33. An alanine was added to the N-terminus of the defensin or defensin-like sequence to ensure efficient cleavage by the KEX2 cleavage site. The synthetic genes which comprise DNA encoding from 5′ to 3′ the XhoI site, 3 codons that include the N-terminal alanine codon, and the wild-type defensins of SEQ ID NO: 534, 538, 542, 546, 551, 555, 560, 565, 569, 573, 578, 583, 588, 593, 598, 603, 608, 612, 616, 620, 624, 628, 633, 637, 641, 645, 649, 653, 657, 662, 666, 670, 674, 679, 683, 688, 692, 697, 702, 706, 711, 715, 719, and 723 comprise SEQ ID NO: 780-823 respectively. Derivatives of the SEQ ID NO: 780-823 synthetic DNAs which encode the modified defensins and modified C-terminal fragments set forth in Table 1 or described elsewhere herein are constructed by deleting and/or mutagenizing the corresponding codons of SEQ ID NO: 780-823 or by de novo synthesis. The synthetic genes which comprise DNA encoding from 5′ to 3′ the XhoI site, 3 codons that include the N-terminal alanine codon, and the wild-type defensin-like proteins of SEQ ID NO: 728, 730, 732, and 734 comprise SEQ ID NO: 824, 825, 826, and 827, respectively. Derivatives of the SEQ ID NO: 824, 825, 826, and 827 synthetic DNAs which encode the modified defensin-like proteins and modified C-terminal fragments thereof set forth in Table 1 or described elsewhere herein are constructed by deleting and/or mutagenizing the corresponding codons of SEQ ID NO: 824, 825, 826, and 827 or by de novo synthesis.

The pPICZαA vector containing each defensin or defensin-like sequence was linearized by digestion with SacI or PmeI restriction enzymes and transformed into P. pastoris X33 by electroporation. Putative transformants were selected on yeast extract peptone dextrose medium (YPD) agar plates containing 150 μg/mL of zeocin, followed by inoculation into 100 μl of YPD broth containing 500 μg/mL of zeocin. Transformants that survived at higher zeocin concentrations and with higher optical density (OD600) readings were selected and used for small-scale expression testing of defensin or defensin-like peptides. Selected transformants were grown for 2 days in 2 ml buffered minimal glycerol (BMG, Invitrogen) media at 30 °C on a shaker at 225 rpm. Cells were harvested by centrifugation at 4,000 rpm for 10 minutes and re-suspended in 2 ml of buffered minimal methanol (BMM, Invitrogen) media to induce peptide expression. The cultures were grown for 4 days at 30 °C and 1% (v/v) methanol was added every 24 h to maintain the induction. After 4 days, cells were harvested, and 20 μl of supernatant were collected and run on SDS-PAGE gel for the expression of recombinant peptide. High expression transformants were selected based on the intensities of peptide bands.

Defensin or defensin-like peptides were purified using CM Sephadex C-25 cation-exchange chromatography. Briefly, P. pastoris X33 transformants containing defensin or defensin-like peptides were grown for 2-3 days in 100 ml of buffered minimal glycerol (BMG, Invitrogen) media at 30 °C on a shaker at 225 rpm. Cells were harvested by centrifugation at 4,000 rpm at room temperature (RT) for 10 minutes and re-suspended in 100 ml of buffered minimal methanol (BMM, Invitrogen) media to induce peptide expression. The cultures were grown for 4 days at 30 °C at 225 rpm and 1% (v/v) methanol was added every 24 h to maintain the induction. After induction, cells were harvested by centrifugation at 4,000 rpm at 4 °C for 10 minutes, and the supernatant was collected and filtered through 0.22 μm filters to further eliminate cellular debris. The cation-exchange resin (CM-Sephadex C-25, Cat no: C25120, Sigma) previously equilibrated with binding buffer (25 mM Sodium Acetate Anhydrous, pH 5.2/6.0) was added to the supernatant and incubated overnight at 4 °C at 100 rpm. Samples were loaded onto gravity columns (Poly-Prep® Chromatography Columns, Cat no: 7311550, Bio-Rad) and after washing the resin with a binding buffer, the bound proteins were eluted with the elution buffer (1M NaCl, 50 mM Tris, pH 7.6). The fractions containing each defensin or defensin-like peptides were concentrated and dialyzed against 10 mM Tris, pH 7.6 using an 1 kD/3 kD centrifugal filter units. The purity and size of each peptide was determined by SDS-PAGE gel electrophoresis and the peptide concentration was determined by BCA assay.

### Example 6. Antifungal Assays

The antifungal activity of defensin and defensin-like peptides will be determined in an in vitro assay using 96-well microtiter plates on an Opentron liquid handler. Briefly, forty-five microliters of each protein dilution (0, 0.375, 0.75, 1.5, 3, 6 and 12 μM) will be added to each well of the microtiter plate containing 45 μL of (~10⁵ spores/ml) fungal spore suspension (e.g., F. graminearum, B. cinerea, and Z. tritici). The plates will be incubated at room temperature and the quantitative fungal growth inhibition will be estimated by measuring the absorbance at 595 nm using a Tecan Infinite M200 Pro (Tecan Systems Inc., San Jose, CA) microplate reader at 48 hrs. The fungal cell viability will be determined by the resazurin cell viability assay. After incubation at 48 hrs, 0.1% resazurin solution will be added to each well and the plate re-incubated overnight. A change from blue to pink color indicates reduction of resazurin and cell viability.

### Example 7. Expression, Purification, and In Vitro Antifungal Assays of Peptides

Expression and purification of the defensin and defensin-like peptide was performed essentially as described in Example 5. Selected transformants were grown for 2 days in 2 ml buffered minimal glycerol (BMG, Invitrogen) media at 28-30 °C on a shaker at 225 rpm. Cells were harvested by centrifugation at 4,000 rpm for 10 minutes and re-suspended in 2 ml of buffered minimal methanol (BMM, Invitrogen) media to induce peptide expression. The cultures were grown for 4 days at 28-30 °C and 1% (v/v) methanol was added every 24 h to maintain the induction. The 100 ml cultures were grown for 4 days at 28-30 °C at 225 rpm and 1% (v/v) methanol was added every 24 h to maintain the induction.

The Pichia pastoris X33 expression system was effectively utilized for the expression and purification of defensin and defensin-like peptides. The yields of these peptides varied significantly, ranging from no detectable expression to a maximum of 4.47 mg per 100 ml of culture volume. A summarization of the purification yield is presented in Table 20 for all the peptides.

TABLE 20

| Peptide ID | SEQ ID NO ¹ | Total Yield (mg/100 ml) |
|---|---|---|
| PD81.1.1 | 534 | 0.51 |
| PD82.1.1 | 538 | 1.19 |
| PD83.1.1 | 542 | 0.31 |
| PD84.1.1 | 546 | 0.76 |
| PD85.1.1 | 551 | 0.83 |
| PD86.1.1 | 555 | 1.34 |
| PD87.1.1 | 560 | 1.29 |
| PD88.1.1 | 565 | 0.31 |
| PD89.1.2 | 569 | 0.82 |
| PD90.1.1 | 573 | 1.67 |
| PD91.1.1 | 578 | 0.30 |
| PD92.1.1 | 583 | 1.20 |
| PD93.1.1 | 588 | 3.01 |
| PD94.1.1 | 593 | 1.11 |
| PD95.1.1 | 598 | 0.32 |
| PD96.1.1 | 603 | 1.78 |
| PD97.1.1 | 608 | 0.72 |
| PD98.1.1 | 612 | 0 |
| PD99.1.1 | 616 | 0.34 |
| PD100.1.1 | 620 | 2.55 |
| PD101.1.1 | 624 | 1.12 |
| PD102.1.1 | 628 | 1.61 |
| PD103.1.1 | 633 | 1.59 |
| PD104.1.1 | 637 | 0.74 |
| PD105.1.1 | 641 | 0.86 |
| PD106.1.1 | 645 | 3.23 |
| PD107.1.1 | 649 | 2.33 |
| PD108.1.1 | 653 | 0.69 |
| PD109.1.1 | 657 | 1.59 |
| PD110.1.1 | 662 | 2.52 |
| PD111.1.1 | 666 | 0.64 |
| PD112.1.1 | 670 | 0.23 |
| PD113.1.1 | 674 | 4.47 |
| PD114.1.1 | 679 | 0.30 |
| PD115.1.1 | 683 | 1.63 |
| PD116.1.1 | 688 | 3.54 |
| PD117.1.1 | 692 | 1.04 |
| PD118.1.1 | 697 | 1.91 |
| PD119.1.1 | 702 | 1.39 |
| PD120.1.1 | 706 | 0.42 |
| PD121.1.1 | 711 | 2.37 |
| PD122.1.1 | 715 | 0.90 |
| PD123.1.1 | 719 | 0.68 |
| PD124.1.1 | 723 | 3.42 |
| PD125.1.1 | 728 | 2.26 |
| PD126.1.1 | 730 | 1.25 |
| PD127.1.1 | 732 | 1.71 |
| PD128.1.1 | 734 | 1.00 |

¹ SEQ ID NO of the defensin peptide lacking the N-terminal alanine

The antifungal activity of defensin and defensin-like peptides were determined in an in vitro assay using 96-well microtiter plates on an Opentron liquid handler. Briefly, forty-five microliters of each protein dilution (0, 0.187, 0.375, 0.75, 1.5, 3, 6, or 12 μM) were added to each well of the microtiter plate containing 45 μL of (~10⁵ spores/ml) fungal spore suspension (e.g., Fusarium graminearum PH-1, Botrytis cinerea T4, and Zymoseptoria tritici IPO323). The plates were incubated at room temperature, and quantitative fungal growth inhibition was estimated by measuring the absorbance at 595 nm at 48 hours for F. graminearum and B. cinerea, and at 72 hours for Z. tritici. Fungal cell viability was determined using the resazurin cell viability assay. After a 48-hour incubation, a 0.05% resazurin solution was added to each well for F. graminearum and B. cinerea. For Z. tritici IPO323, a 0.025% resazurin solution was added at the 72 hours. Subsequently, the plates were re-incubated overnight for F. graminearum and B. cinerea, and up to day 14 for Z. tritici. A change from blue to pink/colorless signals resazurin reduction and indicates metabolically active fungal spores.

The in-vitro antifungal assays revealed that peptides inhibited the growth of F. graminearum PH-1, B. cinerea T4, and Z. tritici IPO323. The MIC values for the in-vitro antifungal activity of each peptide against these pathogens are shown in Tables 21 and 22.

TABLE 21

| | | F. | |
|---|---|---|---|
| | | graminearum | |
| | | PH-1 | B. cinerea T4 |
| | | MIC Day1 | MIC Day1 |
| Peptide ID | SEQ ID NO ¹ | (μM) | (μM) |
| Amphotericin-B | | 6 | 3 |
| PD81.1.1 | 534 | 1.5 | 1 |
| PD82.1.1 | 538 | 1.5 | 1.125 |
| PD83.1.1 | 542 | >12 | >12 |
| PD84.1.1 | 546 | >12 | >12 |
| PD85.1.1 | 551 | >12 | >12 |
| PD86.1.1 | 555 | 6 | 12 |
| PD87.1.1 | 560 | 0.75 | 0.375 |
| PD88.1.1 | 565 | >12 | 0.75 |
| PD89.1.2 | 569 | 12 | 2.5 |
| PD90.1.1 | 573 | 5 | 0.75 |
| PD91.1.1 | 578 | >12 | >12 |
| PD92.1.1 | 583 | 3 | 3 |
| PD93.1.1 | 588 | >12 | 6 |
| PD94.1.1 | 593 | >12 | >12 |
| PD95.1.1 | 598 | 0.75 | 0.75 |
| PD96.1.1 | 603 | 0.75 | 6 |
| PD97.1.1 | 608 | 1.5 | 1.25 |
| PD98.1.1 | 612 | ND | ND |
| PD99.1.1 | 616 | 0.75 | 0.75 |
| PD100.1.1 | 620 | 6 | 6 |
| PD101.1.1 | 624 | 1.5 | 1.5 |
| PD102.1.1 | 628 | 1.5 | 1.25 |
| PD103.1.1 | 633 | 2.5 | 1.75 |
| PD104.1.1 | 637 | >12 | >12 |
| PD105.1.1 | 641 | ND | ND |
| PD106.1.1 | 645 | 0.75 | 1 |
| PD107.1.1 | 649 | ND | ND |
| PD108.1.1 | 653 | >12 | >12 |
| PD109.1.1 | 657 | ND | ND |
| PD110.1.1 | 662 | ND | ND |
| PD111.1.1 | 666 | ND | ND |
| PD112.1.1 | 670 | ND | ND |
| PD113.1.1 | 674 | 6 | 6 |
| PD114.1.1 | 679 | >12 | >12 |
| PD115.1.1 | 683 | >12 | >12 |
| PD116.1.1 | 688 | ND | ND |
| PD117.1.1 | 692 | >12 | >12 |
| PD118.1.1 | 697 | 1.5 | 1.5 |
| PD119.1.1 | 702 | 1.5 | 4.5 |
| PD120.1.1 | 706 | 12 | >12 |
| PD121.1.1 | 711 | 1.5 | 2 |
| PD122.1.1 | 715 | 1.5 | 0.75 |
| PD123.1.1 | 719 | 6 | 6 |
| PD124.1.1 | 723 | 2 | 0.75 |
| PD125.1.1 | 728 | 8 | 10 |
| PD126.1.1 | 730 | >12 | >12 |
| PD127.1.1 | 732 | >12 | >12 |
| PD128.1.1 | 734 | >12 | 3 |

¹ SEQ ID NO of the defensin peptide lacking the N-terminal alanine

TABLE 22

| | | Z. tritici IPO323 | | |
|---|---|---|---|---|
| | SEQ ID | MIC Day 1 | MIC Day 7 | MIC Day 14 |
| Peptide ID | NO ¹ | (μM) | (μM) | (μM) |
| Ampho- | | 0.84 | 5.44 | 8.92 |
| tericin-B | | | | |
| PD81.1.1 | 534 | 0.5 | 0.75 | 3 |
| PD82.1.1 | 538 | 0.625 | 2 | 3.5 |
| PD83.1.1 | 542 | ND | ND | ND |
| PD84.1.1 | 546 | >12 | >12 | >12 |
| PD85.1.1 | 551 | 0.625 | 1.75 | 10 |
| PD86.1.1 | 555 | 7 | 12 | >12 |
| PD87.1.1 | 560 | 0.375 | 1.5 | 2.5 |
| PD88.1.1 | 565 | 1.5 | 3 | 4 |
| PD89.1.2 | 569 | 1.5 | 6 | 12 |
| PD90.1.1 | 573 | 0.625 | 1.25 | 2 |
| PD91.1.1 | 578 | 6 | >6 | >6 |
| PD92.1.1 | 583 | 0.75 | 1.75 | 2.5 |
| PD93.1.1 | 588 | 2 | 5 | 8 |
| PD94.1.1 | 593 | >12 | >12 | >12 |
| PD95.1.1 | 598 | ND | ND | ND |
| PD96.1.1 | 603 | 2.5 | 10 | >12 |
| PD97.1.1 | 608 | 0.75 | 1.5 | 1.5 |
| PD98.1.1 | 612 | ND | ND | ND |
| PD99.1.1 | 616 | ND | ND | ND |
| PD100.1.1 | 620 | 1 | 4 | 8 |
| PD101.1.1 | 624 | 0.75 | 1.5 | 1.5 |
| PD102.1.1 | 628 | 1 | 1.5 | 2.5 |
| PD103.1.1 | 633 | 1.25 | 1.25 | 3 |
| PD104.1.1 | 637 | >12 | >12 | >12 |
| PD105.1.1 | 641 | ND | ND | ND |
| PD106.1.1 | 645 | 0.375 | 1.5 | 2.5 |
| PD107.1.1 | 649 | ND | ND | ND |
| PD108.1.1 | 653 | >12 | >12 | >12 |
| PD109.1.1 | 657 | ND | ND | ND |
| PD110.1.1 | 662 | ND | ND | ND |
| PD111.1.1 | 666 | ND | ND | ND |
| PD112.1.1 | 670 | ND | ND | ND |
| PD113.1.1 | 674 | 4 | 12 | >12 |
| PD114.1.1 | 679 | ND | ND | ND |
| PD115.1.1 | 683 | >12 | >12 | >12 |
| PD116.1.1 | 688 | ND | ND | ND |
| PD117.1.1 | 692 | >12 | >12 | >12 |
| PD118.1.1 | 697 | 1.5 | 2 | 3 |
| PD119.1.1 | 702 | 2.5 | 6 | 12 |
| PD120.1.1 | 706 | ND | ND | ND |
| PD121.1.1 | 711 | 1 | 4 | 5 |
| PD122.1.1 | 715 | 0.75 | 2.5 | 3 |
| PD123.1.1 | 719 | 3 | 5 | 10 |
| PD124.1.1 | 723 | 0.75 | 3.25 | 4 |
| PD125.1.1 | 728 | 3 | 7 | 9 |
| PD126.1.1 | 730 | >12 | >12 | >12 |
| PD127.1.1 | 732 | >12 | >12 | >12 |
| PD128.1.1 | 734 | 3 | 7 | 8 |

¹ SEQ ID NO of the defensin peptide lacking the N-terminal alanine

In the case of F. graminearum, PD87.1.1, PD95.1.1, PD96.1.1, PD99.1.1 and PD106.1.1 exhibited potent in-vitro antifungal activity against F. graminearum at sub-micromolar concentrations, specifically at 0.75 μM, with some peptides showing activity ranging from 1 to greater than 12 μM. The positive control, Amphotericin-B, showed MIC value of 6 μM against F. graminearum.

In the case of Botrytis cinerea, PD87.1.1, PD88.1.1, PD90.1.1, PD95.1.1, PD99.1.1, PD122.1.1, and PD124.1.1 exhibited potent in-vitro antifungal activity against B. cinerea at sub-micromolar concentrations (0.375 to 0.75 μM). PD81.1.1 and PD106.1.1 exhibited in-vitro antifungal activity against B. cinerea at 1 μM, with some peptides showing activity ranging from 1.5 to greater than 12 μM. The positive control, Amphotericin-B, showed MIC value of 3 μM against B. cinerea.

For Z. tritici, MIC values were assessed on days 1, 7, and 14. Stability was indirectly assessed by observing changes in MIC values over time. PD97.1.1 and PD101.1.1 exhibited an initial MIC value of 0.75 μM on day 1. Subsequent measurements on days 7 and 14 revealed an MIC value of 1.5 μM, suggesting a relative stability in their antifungal efficacy over the assessed time period. PD81.1.1, PD82.1.1, PD87.1.1, PD90.1.1, PD92.1.1, PD102.1.1, PD103.1.1, PD106.1.1, PD118.1.1, and PD122.1.1 exhibited the MIC values between 2 and 3.5 μM on day 14. Some peptides exhibited MIC values between 4 and 12 μM on day 14, while others demonstrated values greater than 12 μM, indicating variability in the stability of antifungal activity among the different peptides. The positive control, Amphotericin-B, showed MIC value of 0.84 μM on day 1, 5.44 μM on day 7, and 8.92 μM on day 14 against Z. tritici.

### Example 8. In Planta Antifungal Activity

Pepper—Botrytis cinerea in Planta Assays

The pepper (Capsicum annuum cv. California Wonder) plants were grown in a greenhouse for four weeks under a 14/10 h light/dark cycle, with day temperatures of 28 °C and night temperatures of 22 °C and a relative humidity of 50%.

For the preventative activity assessment, a 24 μM solution of PD122.1.1 (SEQ ID NO: 715 with an N-terminal alanine) containing 0.02% Tween was sprayed onto treatment pepper plants at a volume of 2 mL/plant. The control plants were set up similarly, but without peptides. After 24 hours, B. cinerea spores (~7×10⁴) suspended in 0.5×SFM were sprayed onto the treatment and control pepper plants at a volume of 1 mL/plant. Plants were placed in a highly humid environment at room temperature. Disease symptoms were observed at 96 HPI.

For the curative activity assessment, B. cinerea spores (~7×10⁴) suspended in 0.5×SFM were sprayed onto the treatment and control pepper plants at a volume of 1 mL/plant. After 24 hours, a 24 μM solution of PD122.1.1 containing 0.02% tween, was sprayed onto treatment pepper plants at a volume of 2 mL/plant. The control plants were set up similarly, but without peptides. Plants were placed in a highly humid environment at room temperature. Disease symptoms were observed at 5 DPI.

The potential of PD122.1.1 to offer both preventative and curative protection against B. cinerea (gray mold disease) was evaluated by spraying four-week-old pepper plants with 24 μM PD122.1. Application of PD122.1.1 resulted in a reduction of gray mold disease symptoms compared to the control plants, which were only inoculated with the pathogen (as shown in FIGS. 3A and 3B). These results demonstrate that the spray application of PD122.1.1 effectively protects pepper plants against gray mold disease, offering preventative protection (FIG. 3A) at 96 hours post-inoculation (hpi) and curative benefits (FIG. 3B) at 5 days post-inoculation (dpi).

Wheat—Zymoseptoria tritici in Planta Assays

The wheat (Triticum aestivum cv. Bobwhite) plants were grown in a controlled growth chamber for two weeks under a 16 h/8 h light/dark cycle, with temperatures of 20 °C, light intensity of 200 μMol and a relative humidity of 70%.

For the curative activity assessment, spores of Z. tritici (1×10⁶) suspended in sterile water containing 0.1% Tween were sprayed onto both treatment and control wheat plants at a volume of 2 mL/plant. After 24 hours, the treatment wheat plants were sprayed with a 48 μM solution of PD101.1.1 (SEQ ID NO: 624 with an N-terminal alanine), containing 0.02% Tween, at a volume of 2 mL/plant. The control plants were set up similarly, but without peptides. All plants were placed in a highly humid environment under a 16/8 h light/dark cycle, with day and night temperatures of 20 °C, a light intensity of 200 μMol, and a relative humidity of 80-95%. Disease symptoms were observed at 21 DPI.

The potential of PD101.1.1 to offer curative protection against Z. tritici (Septoria tritici blotch (STB)) was evaluated by spraying two-week-old wheat plants with 48 μM PD101.1. Application of PD101.1.1 resulted in a reduction of STB disease symptoms compared to the control plants, which were only inoculated with the pathogen (as shown in FIG. 4). These results demonstrate that the spray application of PD101.1.1 effectively protects wheat plants against STB disease, offering curative protection at 21 days post-inoculation (dpi).

The breadth and scope of the present disclosure should not be limited by any of the above-described examples.

What is claimed is:

1. A peptide comprising the amino acid sequence of a modified defensin or modified defensin-like peptide wherein the wild-type gamma-core consensus peptide GXCX3-9C or GXCX3-22C of the wild-type defensin or wild-type defensin-like peptide is replaced by a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M) (F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22(F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), and wherein the peptide has a net positive charge of at least 3 and a hydrophobic amino acid content at least 18%.

2. The peptide of claim 1, wherein the wild-type defensin comprises the polypeptide of SEQ ID NO: 11-41, 81 to 533, 534, 538, 542, 546, 551, 555, 560, 565, 569, 573, 578, 583, 588, 593, 598, 603, 608, 612, 616, 620, 624, 628, 633, 637, 641, 645, 649, 653, 657, 662, 666, 670, 674, 679, 683, 688, 692, 697, 702, 706, 711, 715, 719, 723, 743, 746, 749, or 752 or wherein the wild-type defensin-like peptide comprises the polypeptide of SEQ ID NO: 728, SEQ ID NO: 730, SEQ ID NO: 732, or SEQ ID NO: 734.

3. The peptide of claim 1, wherein the modified defensin peptide has at least 70%, 75%, 80%, 85%, 90%, 95%, 98%, 99%, or 99.5% sequence identity to SEQ ID NO: 536, 540, 544, 549, 553, 558, 563, 567, 571, 576, 581, 586, 591, 596, 601, 606, 610, 614, 618, 622, 626, 631, 635, 639, 643, 647, 651, 655, 660, 664, 668, 672, 677, 681, 686, 690, 695, 700, 704, 709, 717, 721, 726, 745, 748, 751, 754, or 756.

4. The peptide of claim 3, wherein the modified defensin peptide or modified defensin-like peptide comprises SEQ ID NO: 535, 536, 537, 539, 540, 541, 543, 544, 545, 547, 548, 549, 550, 552, 553, 554, 556, 557, 558, 559, 561, 562, 563, 564, 566, 567, 568, 570, 571, 572, 574, 575, 576, 577, 579, 580, 581, 582, 584, 585, 586, 587, 589, 590, 591, 592, 594, 595, 596, 597, 599, 600, 601, 602, 604, 605, 606, 607, 609, 610, 611, 613, 614, 615, 617, 618, 619, 621, 622, 623, 625, 626, 627, 629, 630, 631, 632, 634, 635, 636, 638, 639, 640, 642, 643, 644, 646, 647, 648, 650, 651, 652, 654, 655, 656, 658, 659, 660, 661, 663, 664, 665, 667, 668, 669, 671, 672, 673, 675, 676, 677, 678, 680, 681, 682, 684, 685, 686, 687, 689, 690, 691, 693, 694, 695, 696, 698, 699, 700, 701, 703, 704, 705, 707, 708, 709, 710, 712, 713, 714, 716, 717, 718, 720, 721, 722, 724, 725, 726, 727, 728, 729, 730, 731, 732, 733, 734, 735, 736, 744, 745, 747, 748, 750, 751, 753, 754, 755, 756, or a variant thereof comprising a deletion of 1 to 10 N-terminal amino acid residues, a deletion of 1 to 10 C-terminal amino acid residues, and/or a conservative amino acid substitution of 1 to 10 amino acid residues, wherein the modified gamma-core consensus peptide is conserved in the variant.

5. A peptide comprising the amino acid sequence of a modified defensin peptide fragment wherein the wild-type gamma-core consensus peptide of GXCX3-9C or GXCX3-22C of the corresponding wild-type defensin peptide fragment is replaced by a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M) (F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22(F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), wherein the peptide further comprises a second C-terminal cysteine residue located C-terminal to the cysteine residue in the modified gamma-core consensus sequence, wherein the peptide has a net positive charge of at least 3 and a hydrophobic amino acid content at least 18%, and optionally wherein the peptide comprises, essentially consists, or consists of: (i) 30 amino acid residues or less; or (ii) 15, 16, or 17 to 30 amino acid residues.

6. The peptide of claim 5, wherein said peptide comprises a modified C-terminal fragment of a wild-type defensin peptide, said modification comprising the substitution of the wild-type gamma-core consensus peptide of GXCX3-9C or GXCX3-22C of the wild-type defensin C-terminal fragment with the modified gamma-core consensus peptide, optionally wherein said peptide contains one additional cysteine residue C-terminal to the modified gamma-core consensus peptide sequence, and optionally wherein the peptide comprises a disulfide bond between the two cysteine residues in the modified C-terminal fragment having the one additional cysteine residue.

7. The peptide of claim 6, wherein said peptide comprises, consists essentially of, or consists of SEQ ID NO: 537, 541, 545, 550, 554, 559, 564, 568, 572, 577, 582, 587, 592, 597, 602, 607, 611, 615, 619, 623, 627, 632, 636, 640, 644, 648, 652, 656, 661, 665, 669, 673, 678, 682, 687, 691, 696, 701, 705, 710, 714, 718, 722, 727, or a variant thereof comprising a conservative amino acid substitution of 1 to 2, 3, 4, or 5 amino acid residues, wherein the modified gamma-core consensus peptide is conserved in the variant.

8. A peptide comprising a C-terminal fragment of a defensin-like peptide, wherein said C-terminal fragment lacks 1 to 35 amino terminal amino acids of the corresponding wild-type defensin-like peptide and/or comprises a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M)(F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22 (F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M).

9. A peptide comprising, consisting essentially of, or consisting of SEQ ID NO: 729, 731, 733, 735, 736, 828, 829, 830, 831, 832, or 833, a variant thereof having comprising a conservative amino acid substitution of 1 to 2, 3, 4, or 5 amino acid residues, or a variant thereof having at least 90% or 95% sequence identity thereto, wherein the gamma-core consensus peptide is conserved in the variants or wherein the gamma-core consensus peptide is replaced with a modified gamma-core consensus peptide comprising the peptide sequence GXCX3-9(F/W/Y), GXCX3-9(F/W/Y/L/V/I/M), GXCX3-9(F/W/Y)(F/W/Y/M)(F/W/Y), GXCX3-9(F/W/Y/L/V/I/M)(F/W/Y/L/V/I/M)(F/W/Y/L/V/I/M), GXCX9-22(F/W/Y), GXCX16-22(F/W/Y), GXCX9-22(F/W/Y/L/V/I/M), GXCX16-22(F/W/Y/L/V/I/M), GXCX9-22(F/W/Y)(F/W/Y/L/V/I/M)(F/W/Y), GXCX16-22(F/W/Y)(F/W/Y/L/V/I/M)(F/W/Y), GXCX9-22(F/W/Y/L/V/I/M)(F/W/Y/L/V/I/M)(F/W/Y/L/V/I/M), GXCX16-22 (F/W/Y/L/V/I/M)(F/W/Y/L/V/I/M)(F/W/Y/L/V/I/M), GXCX3-9(F/W/Y)(R/K/H)(F/W/Y), GXCX3-9(F/W/Y/L/V/I/M)(R/K/H)(F/W/Y/L/V/I/M), GXCX9-22(F/W/Y)(R/K/H)(F/W/Y), GXCX16-22(F/W/Y)(R/K/H)(F/W/Y), GXCX16-22(F/W/Y/L/V/I/M)(R/K/H)(F/W/Y/L/V/I/M), or GXCX9-22(F/W/Y/L/V/I/M)(R/K/H)(F/W/Y/L/V/I/M), optionally wherein the gamma-core consensus peptide comprises the peptide sequence GXCX3-9C or GXCX9-16C.

10. A composition comprising the peptide of any one of claims 1 to 9 and an agriculturally, pharmaceutically, or veterinary-practicably acceptable carrier, diluent, or excipient.

11. The composition of claim 10, wherein the peptide is provided at a concentration of about 0.1, 0.5, 1.0, or 5 μg/ml to about 1, 5, 20, 50, or 100 mg/ml or at a concentration of about 0.1, 0.5, 1.0, or 5 μg/gram to about 1, 5, 20, 50, or 100 mg/gram and optionally wherein the composition comprises a sodium salt at a concentration of at least 100 mM and/or a calcium salt at a concentration of at least 2 mM.

12. A method for preventing or reducing crop damage by a plant pathogenic microbe comprising the step of contacting a plant, a plant seed, or other part of said plant with an effective amount of the composition of claim 10.

13. The method of claim 12, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp., Magnaporthe sp., or Stenocarpella sp.

14. A medical device comprising the device and the composition of claim 4, wherein the device comprises at least one surface that is topically coated and/or impregnated with the composition.

15. The medical device of claim 14, wherein said device is a stent, a catheter, a contact lens, a condom, a patch, or a diaphragm.

16. A method for treating, preventing, or inhibiting a microbial infection in a subject in need thereof comprising administering to said subject an effective amount of the composition of claim 10.

17. The method of claim 16, wherein said administration comprises topical, enteral, parenteral, and/or intravenous introduction of the composition.

18. The method of claim 16, wherein the subject is a human, livestock, poultry, fish, or a companion animal.

19. The method of claim 16, wherein the microbial infection is of a mucosal membrane, eye, skin, and/or a nail and the composition is applied to the mucosal membrane, eye, skin, and/or nail.

20. The method of claim 16, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

21. The method of claim 16, wherein the microbial infection is by an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, or Blastomyces species.

22. The method of claim 16, wherein the microbial infection is by a Candida species and wherein the Candida species is Candida albicans (C. albicans), C. auris, C. glabrata, C parapsilosis, C. tropicalis, or C. krusei.

23. The composition of claim 10 for use in a method of treating, preventing, or inhibiting microbial infection in a subject in need thereof.

24. The composition of claim 23, wherein the subject is a human, livestock, poultry, fish, or a companion animal.

25. A plant part that is at least partly coated with the composition of claim 10.

26. The plant part of claim 25, wherein the part is a seed and the seed is optionally a corn, soybean, wheat, rice, cotton, Brassica sp., or tomato seed.

27. The plant part of claim 25, wherein the plant part is a leaf, stem, fruit, vegetable, root, tuber, or flower.

28. A recombinant polynucleotide comprising a polynucleotide encoding a peptide comprising the peptide of any one of claims 1 to 9, wherein the polynucleotide encoding the peptide is operably linked to a polynucleotide comprising a promoter which is heterologous to the polynucleotide encoding the peptide.

29. A recombinant polynucleotide encoding a peptide comprising: (i) a defensin peptide comprising a long gamma-core consensus sequence GXCX16-22C, optionally wherein the defensin peptide comprises SEQ ID NO: 578, 608, 612, or a variant thereof having at least 70%, 75%, 80%, 85%, 90%, 95%, 98%, or 99% sequence identity to SEQ ID NO: 578, 608, or 612, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues; or (ii) a defensin or defensin-like peptide comprising a wild-type gamma-core consensus peptide GXCX₃₋₉C, GXCX₉₋₁₆C or GXCX₃₋₂₂C, optionally wherein the defensin or defensin-like peptide comprises SEQ ID NO: 534, 538, 565, 569, 573, 583, 598, 603, 616, 624, 628, 633, 645, 697, 702, 711, 715, 723, 728, 729, 730, 731, 732, 733, 734, 735, 736, or a variant thereof having at least 70%, 75%, 80%, 85%, 90%, 95%, 98%, or 99% sequence identity to SEQ ID NO: 534, 538, 565, 569, 573, 583, 598, 603, 616, 624, 628, 633, 645, 697, 702, 711, 715, 723, 728, 729, 730, 731, 732, 733, 734, 735, 736, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues; wherein the polynucleotide encoding the peptide is operably linked to a polynucleotide comprising a promoter which is heterologous to the polynucleotide encoding the peptide

30. The recombinant polynucleotide of claim 28 or 29, wherein the recombinant polynucleotide further comprises a polynucleotide encoding: (i) a transit peptide, a vacuolar targeting peptide, and/or an endoplasmic reticulum targeting peptide; (ii) a plastid targeting peptide; and/or (iii) a polyadenylation or transcriptional termination signal, wherein the polynucleotides of (i), (ii), and/or (iii) are operably linked to the polynucleotide encoding the antimicrobial peptide.

31. The recombinant polynucleotide of claim 28 or 29, wherein the polynucleotide encoding the peptide is inserted into a heterologous nuclear or plastid genome of a cell and operably linked to an endogenous promoter located in the heterologous nuclear or plastid genome.

32. A plant nuclear or plastid genome comprising a polynucleotide encoding a peptide comprising the peptide of claims 1 to 9, wherein the polynucleotide is heterologous to the nuclear or plastid genome and wherein the polynucleotide is operably linked to an endogenous promoter of the nuclear or plastid genome.

33. A cell comprising the recombinant polynucleotide of claim 28 or 29, wherein the cell is optionally a bacterial, yeast, or plant cell.

34. A plant comprising the recombinant polynucleotide of claim 28 or 29.

35. A plant part of the plant of claim 34, wherein the plant part comprises the recombinant polynucleotide, optionally wherein the plant part is a seed, stem, leaf, root, tuber, flower, vegetable, or fruit.

36. A method for producing plant seed that provides plants resistant to infection by a plant pathogenic microbe that comprises the steps of: (i) selfing or crossing the plant of claim 34; and (ii) harvesting seed that comprises the recombinant polynucleotide of the plant from the self or cross, thereby producing plant seed that provide plants resistant to infection by a plant pathogenic microbe.

37. A method for producing an antifungal peptide comprising: (i) culturing the cell of claim 33 under conditions wherein the peptide, defensin, or defensin-like peptide is expressed by the cell; and (ii) purifying the peptide, defensin peptide, or defensin-like peptide from the culture.

38. The method of claim 37, wherein the cell is a yeast cell, optionally wherein the recombinant polynucleotide comprises a polynucleotide encoding a transit peptide which is operably linked to the polynucleotide encoding the peptide, defensin peptide, or defensin-like peptide and optionally wherein the peptide, defensin peptide, or defensin-like peptide is purified from the culture supernatant.

39. The method of claim 38, wherein the yeast cell is a Candida, Kluveromyces, Hansuela, Pichia, Saccharomyces, Schizosaccharomyces, or Yarrowia cell.

40. An isolated peptide comprising: (i) a defensin peptide comprising a long gamma-core consensus sequence GXCX16-22C, optionally wherein the defensin peptide comprises SEQ ID NO: 578, 608, 612, or a variant thereof having at least 70%, 75%, 80%, 85%, 90%, 95%, 98%, or 99% sequence identity to SEQ ID NO: 578, 608, or 612, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues; or (ii) a defensin or defensin-like peptide comprising a wild-type gamma-core consensus peptide GXCX3-9C, GXCX9-16C or GXCX3-22C, optionally wherein the defensin or defensin-like peptide comprises SEQ ID NO: 534, 538, 565, 569, 573, 583, 598, 603, 616, 624, 628, 633, 645, 697, 702, 711, 715, 723, 728, 729, 730, 731, 732, 733, 734, 735, 736, or a variant thereof having at least 70%, 75%, 80%, 85%, 90%, 95%, 98%, or 99% sequence identity to SEQ ID NO: 534, 538, 565, 569, 573, 583, 598, 603, 616, 624, 628, 633, 645, 697, 702, 711, 715, 723, 728, 729, 730, 731, 732, 733, 734, 735, 736, a variant thereof comprising a deletion of 1 to 10 N-terminal amino acid residues, a deletion of 1 to 10 C-terminal amino acid residues, and/or a variant thereof having a conservative amino acid substitution of 1 to 10 amino acid residues.

41. A composition comprising the peptide of claim 40 and an agriculturally, pharmaceutically, or veterinary-practicably acceptable carrier, diluent, or excipient.

42. A method for preventing or reducing crop damage by a plant pathogenic microbe comprising the step of contacting a plant, a plant seed, or other part of said plant with an effective amount of the composition of claim 41.

43. The method of claim 42, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp., Magnaporthe sp., Stenocarpella sp., or Zymoseptoria sp.

44. A plant or plant part which is at least partially coated with the composition of claim 41.

45. The plant or plant part of claim 44, wherein the plant part is a leaf, seed, or fruit.
